SEQUENCE LISTING (25 sequences)

SEQ ID NO: 1 - DNA, 100140 bp, Mus musculus
  modified_base (27350)..(78168): N = A, C, G OR T/U
ggcaatttctgagaggcaacaggcggcaggtctcagcctagagagggccctgaactactttgctggagtgtccgtcctgggagtggctgctgacccagtccaggagacccatgcctgccatggtccctgggtggaaccatggcaacatcacccgctccaaggcagaggagctactttccagagccggcaaggacgggagcttccttgtgcgtgccagcgagtccatcccccgggcctacgcactctgcgtgctgtgagtacccgtctcctcccaactgtcagatccagggaccactgaggtgtggatccaaagggggaacccctgtaatgggagtttgagttaggtttatgtcataggatggtgggacgtgactggcacttcgttgccctgtggggaggggagaagggggggcagcatctgaggcccactttggaccttggcgttcgagttcagggagcctgtgtcatgacaggcttgtgtggtgctagggctctctgagtgactctgggcctcccctatactgcagccctccatgacctgtggtgccaggggtctccgtgagttcctgtggcaggagcagggacagagtcaggaagaaactcaggcctctctggtggagggtgtattggaatgcattttggtcagctcaagcgtcagtcagggactcagtgaagggcaaccttggcaaaagggtcccctcctccaccctgctagtatgtggtcttagagctaattctatttggggagctgagtccggggtgcatttaatcaggataggattcctcgtgtacacattttactctaggctgcaaggacacaggaagccacagagctgctctctgagtaggtctctgtccctccttgcactcagctatgtccctccctatccaggtctctgcccccgttggtacccccccacagcccagtgtgaagatgttgctgaatatgctggttatcctaacaacagaggaaacagccacttgctgaaggttcctttttaacactccgtccgtctagcgtcttctgaggaaggccgccacccctatagtcctgtggtcagaccctgtccaggcttcaggctggagcagggcaggaacactgtcaggaagggtgtgcctacttgaacagcacaggtaccatctgatagattgtccctggactgagagaaagatctttcagagcagctagctgccccccccccaatcttcatgcaggagggaagtgggtgactgaccacagactgttctgagctctgactcatgttgggacctgctgtgctaggcatttgttggctagtttcatcttcaagagagccaggagtgtgggttctatgaacacctagacctgcgattaaggaccatgagggctggacaggttacaaacatcgaggataaggtcggtcatttgctcccagagaccactagcttgtgctgcctacctcctaccaccatcaggctaggcatgccagtggacttgaatggaagtaagaggagctagagtgttcacagagttgggggcaggggcactagaagcccgtacaggctcacggctggctgagcatcttgtgctggtggagttagcagccagcttcctgcacacccaccagcatatttcaggagaagcactgcaggtggcctggacctccccaaagcactgtctcttggggaatctaccagtgagaggccctgaaaggaaagagggcaggaaggtacttttcagtgtgtcacaagctcagcctgactctactaacccagttatatttttttctgtcccagtgggatttgggccaagggcaattctgttgggagttggctatgggctgagagactgttgtatgcattggttacaaatgcacaccacggtatgggttctcttgtgctaaactggcagcatctggaaggctggagtcagagcagaaggagcctgagccaagaccaagggatcttggaggaccttcgggcaacacagaccttgccttttttcttcatctgactcccctgcctcagctgtcttaagtccaagcaaagcaaagatgacacggagattttcaaactaaaggaatgactaccacaacctcagtgttctataatgaccccccccacacacacacacaccctaaaaatgtaggaaaggcaggacagtggtggtgcacacctttaatcccagcactcgggaggcagaggcaggcagatttctgagttcgaggccagcctgatctacaaagtgagctccaggacagccagggctacacagagaaaccctgtctcgaaaaaaaacaaaaaaacaaaaaatgtaggacaggcatactttttaatttgaaaaattgttagaagcctgccttttcatgcaaagagacttaacttcctgaaaaaaacaaactttagatccttactttctcctgttctctgtggatgatggaacctcccccacctcatcgctgccccctcgccatctgccctggccagagtccaggctcctgcccaagaagataagtcagcagcttgtaggacagcaacaaggtcgaggtcagagatggggctgtgaaggagagatggggcatggggtacatgtgggatacagggcagctgagttctctttggtttcaggagtgatagatttggcacgtgtggtgtcttgctggacatcagccagtctgtgtgggctctggggcaggcagctgtgggtcaggtgccgctgggactccatccatgttccttcttgtgaccgaagggacaccaacaggggcccagtgcatgtgttgggtttgtttggccctctgggaagaatgcagcattgtgaggagaaccttcctgctctgagatttgactagacatgacatgggagagggaggtaaatgcttaaagacaaggttgcaattcagttccacccacgtgacacgagtgcacattcaccccacacattgacctttgtttccttcagagagatcaatcctgtcacttatcaccaagcaagaaggcttttctttcttctcagtcatcattcagagagccttggtttaggggagtctggagttacactggggccccggagcactggcctggggagaccttgctagtatgactggagttctgttctaccttccttgaaagggaatgtgtgccttttgagtggggcctgtatcaccttcacttgagtagagcctgtgtcaccttcactcatttgtactcagttcctccgtgatgtcagctcccttcccaggagcctgtgcaccctgttgtggggtattagccaggtggatggagacctattaagagtctcatgagcagggacagcgcagctacaccatgtgttgcagagaagacaatgcttctgagggtagctcagcagatgccgggtttgtgggtcatcccagtagcttcttattgctgaagctacatagcaagaatttgaatgatgacccagcacttggaaacaacttgctctttctaaaagagatgacaaggccagattcaacttggtcaagatgactgttgtctatgtgaatggcatttcccctaaactactctggagtgcttcctcccttgcagggagaatatggttgcctctggtccagccccgatgaaggattccctaagcaagtggtcttccatagtgcacccaggcctggtggtggggtaagctctgtcccagggatagaatgccaatagccttggtagctctggcagtgcagaaagaaaggagaaaaggcatgggacatttacaaccaaaactgccctcagagaaggcattctagtcttttgaaagaaacggtgtgaccagacactgggtgtgataagcctgccaggggagataaaaacaggccctgcttttaggactactggagagcagggtgaagatcacacacttatactgtctcacacttgttctttggtaggaagagaactgcagagaaggagttggtgagggtgaaaatgcctagcagagggagtcagggcatgaggtcatctcccctccccatcctccattgaaaatgtctataaggtttccacagcatgataatggcttttgtgcaaacacagtgtggcactgttttccatatctggcatcaaaggcaaatgagggaaatacctgcataggcagaacccagagctgaaggcctatgggctcctgagaaacatgagaaaaggcctttgtttgagaaggatgctcttgaagactcattgtgtccaagaccaggaggaagggctgaacccaggagggcctatttaaggcctattgtatcatttatgtaagtggcagagtgactatgtttctgcccatacccagtaccctggagctgtcttcccaggtacagggtaggcactggcttaggcgcataggttaaattcacctacaacgcaaggccattagcacttcttaacgatcccatctctctgcctgccacagagatggaacaagaactgctatcgttacccaattcatgccaggccacgtagtcccaaggagcaagactctggagctgcctggaatctcttgtgtcatgtcagcaagacacctagaaccccagctccaaagagaggctggccagaccagttcactggctttacagtgcctcagctgaggttaaggtacccactgttaagtcacccatccacattctagtttgtggttcaatggtccttagtaatggtcacagagttacgcaactgacaccaccatctaatttcagagagttctcattattcccagaagaactgccccccccccccacacacacacatgtgagcagctgcttgcttttcttctgacctctggaaagagccagtctactttctgcctttaaggatttgcctgactcttgcattttgtgtatgtgaaattatatggcgtgcagcttttgacatctggatctgtccacttagcacaatgccctatgctcattgaattgtagcaggcatccaatggtttgataacccactgtgtggacacaccacattccccggttgagtggcgttttgcactgttcttactttttgactgttttgaacaacagttgctgtgaacattcatcaaccaggctctgtgtggatgtctgtttgcaggagtcttggggagtgagaggcagggctgggtcatgtggtgatttttatgtttagcgttttgaggaagtactgaactcttgtgctcagctagagctctaaccagacattgtgagggtccagtttctcttctagttactctctctctctctctctctctctctctctctctctctctctctgtgtgtgtgtgtgtgtgtgtgtgtgtgtgtgtgtgtgtgtgtgtgtgcatgtgtacatttgcacactgtgtgtgtgtcagccaaaagacaacttcaggagttagttcgctccttctaccatgtgggtcccagggactgaactcagatagtcaggcttaatgacaagtacccttaccctctgagctacctcactggccctacctttttcatttctttatttaagacagaggctctcaatgccctggaacttacgtaggttaggtgggctgatgttagaagcaccaagcccagttagtggtggtcggttgcttgttaatgtagtctgggcatcaaactcaggtgcttatgctttcaagggaagcactttatcagctcttacctccccagcccctcacttgtttgtgtgtttgtgagctatggtctcacatagcctaagccagcctcaaactccctattgtagtcaaaattggctttgaactcttgatcatcctgcctccactttctaaatgtattcaccacaatatctggctgtctttttattctacccagcttaggggctgcaaagcagcacagcatctaactgtggttttagtttgtatccctaatgttaataatgctaatgggtcatttgtatgtcttacttggagaactgtctgtatagtctttgcacattgaaatagctatatcatttcaatgataaaataggaagacaggagtaatgtggatattccatagcctaacttgaaacctctaggtgctttgcatatgtaaaaatagagctggccattagtttttagagctgaaagcaaccagtgatatctcagaagcagaagacccatctgtggagaactttccatgacaggagaggagagctgtgacagtgtcacttccgggacttcctggaggcctctgggagacacagagctgtcatgtggggcctccacagaggaagtgctcaaagtgactgaggcaggaagaggacttcaatgacgcaagagtgtctggtggctgctgagccacaggggttgacagcctggaagcctggggagggaggagcctgggtacagacagcaagaaatgcttagaggactgggtatgaagatgaagtctaggagagaggctggcccccaccaccttctgacctgagctccaacttagtaaagagatgccgaagggatgaagtcctctgatcgctaaaatgctagctgttctatgggaggaaacaccatgttggtgggcacttgccctttgagagagagggtgacggaggtggtgagttcaatctccacagcccacatggtgcatgtttgtaagcccagttctaggaaggtggagaccggagggtccctagggttcatgggccagccagactagctgaattagtgagactcagaccatgtctcaaaacaaacaaacaccaaaacaacaacaacaaaccctagagggtatctgaaggaggacatcatgattgtcctctgatctctactcacacattcatgtatgcacacatgtgcccttacacacatacatgggggcatactgttaacatgtgccagcacccatcaatttgggtttactttgcaattgaagtaacactgtgaagggctggccaaaggaacctctgaaaaaagagagtgcccacgtggccctgcagctggaataggcagtgtagaggtggacagacctggtatgaaacagcaaagtcttctttcaggttaacagtagtattggctgtgcggtggtacacacaggtgactgcacactccgaaagctgagcaggaagattgctgaaagtttcagaccagcttcccaggctacatagtgaggccctatctcaacttaaccctcacagtgagttaagcataaagtaaaaagtgtaaaataaacaaaaattgtcacccaatcactaagagactagtgcaagccatgtgaatttgttgatgtatcttctcatacccatttctatcacataatatacatacacacacatacacacacatacacactcacatatatgtacacaccacctttcatttgtggtctggggtttccaacaacatcaatttttcctgcctagttagaatttcttccttaaaatcactcttgctggaagcctaggcttctcttccagtatgtttatgctttatggaatgattctttgattgacatttacattaccagcaatcttgctgtaagtgtggcagcagcaaaaaaggtcttccagcagaattgctacatatgttactggttacaaatccccactgcagttagagcaccagcaggaatcgtcttcaaagactggacacaaaatggcaacttgctttccagaaaaattgacttaatgtatgcttctggcaagcagcatgagaaaatgcccacttccttccaatatcctattctacggaatgttacttttaagaaaattggagtcataacatgaaaggtcatctgtgatcattttgttttgcttttgattgctgatcctgtttgagaggtttgctggccattttcttttgtggatggtcctctattctcttagcttcttttcttcaattaagactgtgtgtagcttgggaagaagggttgtgataagtcccaggctaccacggactgtataatgaggtcttatcttaatagagggtggagtggagctggagatatggcccagtggttaagtgtttgcttagtgtgtgtgaggtcctaggttcaatccccagtaccaaggaatgctgcaagctttgccatctgcctatgtgagttctgtcatgtttgtgacaaatatatcttcacgatggtctgcctttaaggtttagattctctgacatgggagggtcactgttttctactgggacttcttcctgggatttctaacacagagactccttctgcacacagaaatccactcagaaaggttaagactgggtctttgtagctcatccacttgggttaagggaggaacctccagcagtttatcgaattacactgggccccagccttgtcatctgaaaaccaagtataagaggatctatgtggtagaattgttgcatgtgttgatgtgtgtatctgtatcatgtatcatgatactaacaaatgtagccaccgtggctattatttttatatgttttattgttcactcaatttagcttcaatagctaaatcttcttggacttaatagatgacaagtgatgaagatttatgtgtatttttctcaagtaaccatccttccccaaccttttattaaatgacttatgctttttccctccatgattcctttgtctaccatatgctaagctctttctgggttgtttatctgtccaagaatacccaactacaccactacctttacaaagacataagcataatttttcaaaatgttttggggttattatctcttatattcaagtgttttcccaggtaaaccagatagtaatatttttcaacttggacaaaacttactgttatgactttcactgggatggcaggaaatcctaatgtaagctgtgagcttgacagtcttattgtgctccctctctgattgtgtaatatatttcacacagaccctgagtgtttcttgtctgggtctctgctgatgacttgtatgtttttgttattgttgtgaatggcacatttctcccgcagttactggttcatgataagcggttgcctgcatagaattttatgctttcagctttcattttgtcccactctttggccactcctcattcatctttcagctgcttctcctgtcttttctttttaaagagatcttgttatctgtggtgtgtgtgataccatccatgagcacagatgtgtgcgcacacatgtttgtgtttgcctgcacatgggccaggaggtgagatgaggacacctgaaatcatgatccatcactctctgccttgttcacttgagacaatgcttctctctaagcatggggcttgctgattttggctatgctgactgaccaggtccagcaagcctcctgtctctgctacccacccaccccacctcagcactggatttatagatgtgtgtgaccacagctcagttttcttaaattttcagacaggacctcactatgtatccttggctggtttagaaatcacaaagatgggcctacttcccaagatccagtattcaaggcacacaccaccacacagactctctgtcctgctttttcacgtgggctccggggatctaaactcaggtctttatgctcacacagtgagcactcttactcactgaacgttctacccagcccaacttttctgaataggccttcatatcatctgcaaaccaggatcatagtgtttgcttcctaatgcttacaaccactttctcaaatgccatagcctattatattggccagtatttctaaagtggtagagtggcaactgttggaaaaaggaaacgtctgccttaaggagagcttcacataccctgccaccgggttgccttttggtcagggcgttttattacaagaatggaattgggacatgagggttctgggaaggcagatacctgtcagggcctccccatgctgttttgaaacaatgggtttatttaagatagagatagctcctttatatcctttgttgggttggggccctcttttgagtggtgataaggtctggtgattctttttctttttctggcaactggggactgagggaaacagcttcttttagaatcaaaagccattttgcccaacatatcttatgaaagagagacagacaggcagacaggcagaaagaaagaagaaaaagaaaaaggaaaacaccaagatgaatggggatgaaaggacccaccaaaccaacaagacacatgtagtattgggccagtggtaaactcagacagacagacagacagagggctcccttgtgagggcctaactcccgtgtctttgcttaaagcattaggtcaagagtgaccgcttgggtccctcctgctgaggagggttctctccctgtctctcccccacctctcacctgttggccacctcaaccctcttcctgttttctctcttttggaggggcccaaggaagcaggggatagcctctgttaatatatcaagtctcagccctgtgactccagtgttctctaggttggagtagcaagaataaacactcttgcttgctggggcttaggtacattttctctggttctgtgctagccaatatgccttgcctccctctttattggtgcatagggacagggtcactatgtaacccaggttggctttgaactctctatactcctgcttcaacctcccaaatgctagggttgtaggtgcacaggaccatacccacttccatatggctttctgaatgcaatctttccattttgactgttttctacccacctggcccagaagtgtacagcatttatccaagaaaagagaaagcacactgcaaagaatgttttctagaaattagaagtgacacataactagagttctggtgggatttacaagagcagcatgtggagcgagacacagcctggactaaagtttggactaatgttttcataagggtgatggttatgatgctgtcgaatattgacagtgctcaggcagggtcatctgagatacttctgttgggttcagttctgattttggccttaatcgtgtgtggttgtttctagaatatcttttaaaaggccaggcaagccgggcgtggtggtgcacacctttaatcccagcatttgggaggcagaggcaggtgaatttctgagttcaaggccagcctggtctacagagtaagttccaggacagccagggctacacagagaaaccctgtctcaaaaaacaaaaaaataaaaataaaaataaaaggccaggtgggctttttttcctacacttggttaaattttcctttttgggtcagccatatctttatctctctgtcctttctttctgtctgggagcccatgtgtttcaccggctccttagggctattccataagtagctgccctcggctaaggaaagccattctcacgctccctcctttgcttaggaggaagggtcagagctcctgagagataaggccaggtcagatcagggcagagatggctctacaattcccctgcctggctctgtacgggttgagcctaggagccataggggcgtataagtgggcgtggtgaggattctctgtgttccagtgatatctctgagaggcgagatgctatcgggccagatggtggtgatatcgacctggcctgtgaaatgggtggggttggggtggaacattaagttgtgactagagccagaagagcctctcaagagaagctttcccatggggaatgaagcaacgaagtcatgaactctcgaaaaagcattgccccacatttactttaagtatatatgaattcaaaacagtaaaagatcataaagctggctacatagaaaaatataaatgtatacagcaacggaagggaaccaacaaacattgttgaaatgaatatcaaaatccacagtagcatagatcagagtcttgtctgataataagcccaggatgatgtttaaaaaaaaaacaaaaaacaaaaagcaaagagatggaaagggattcaaagaaaaaaaaaacagaaacagccaagaacaaggacaaaccaaacctttctcgtggggagaggcaaattaaggcaagctgccatagttctccccctctcagatctgaaaagagatctgataagccagtcagcaggctgtggggaagtagacactctcatatctgttaggaggaaggaaaaaaaacccgacagactcactggagcgcagctgtcagtctttatcaaaatgtaaaatgccagcaagacatgatggctcatacctgacatcctagcacttgggagactgaggcaggaggattatcttgtgtcagaagccgacctaggctacataaaagtgtgcgtgtccacgcaagcatgtttgtaaagtgcctatgcctttttgtctggctgactctgtctagttcattgtttatccacataactagcaacccctgacacgtgtggtgtccagtgtgagccaggcgctgttccaaaggctttcgatacatggatgcttttagtcttcccagcaacgaatagatatggcattgctgtcatctccattttacaatgaagaaacaaaaacaaagacttcagccatgtatgcatgggttaaccatgtatgacacacggatgtagccatgtatacacacacagatgttgccatgtagccacatatacatggatgtttgttcaccacaacattcttacacaggcaaaacattcaaaataactaccaccaccaggctctggtcaagggcatgacaatacatgtaatgtggtagaatgctgagaacatgagcaaaccatctgtcatgtgtgtcttggttacttccctattgctatgaagagacaccatgaccaaggcaatgtataaaaggatatcttggaattcatgatcatccttaggcttagaatccatgattatcagggcagggaacacggaagcaggcaggtagctgagaacttacatcctgatccttttacaacctcaatgtctacccccagtcacacacctccccaacaaggccaacctcctaatctttcccagttttaccaactgggaaccaagtgtttgaacataggagcctatgagaaacattcgcattcaaaccaccacaatgtggaagactttgatgatattttatgaagaaagcaaactttagaataacataaagagtaagattttatatatttaaaaaatttaaagatatatacacacacatatatacatatatacacatacactcacaatcttttatgcatgtgttttaaaatattttataaataaggataagtctaagaaagtcatggatgataggagatataatttttttcattctatatcttctgtgtgtggtgccttttttcaatatataatctttttttgtttgttttgtgagacagtctcacatagcccaggctggcctccaaccctctatgtaactgaggctaactttgaacttttgattctcttgcctctacctcctgagtactgggattattggtgtatgtcgccattcctggcttatgatgggaactgagcttagggcttcgtgaatgttaggcaaacactaccaaccaagcaaatgcacccagccctcgatataaaacttaaactgagccaggcgtggcgcacgcctttaatcccagcactcaggaggcagaggcaggtggatttctgagttcgaggccagcctggtctacaaagtgagttccaggacagccagggttatagagaaaccctgtctcaaaaaaaacaaaaaacaaaacaaaacaaaacaaaacttaaactgagtttaatagtaactgtgaaattgtaagttaatagtaaatgtgaaattgtagtgttgtgtggcatgtggaagggattagtgaagtgactttttatgaggttgatttttatatcatgggtgtatttcctgcatatatctatgtgcactctgtgcatgcagtgtctgtggagaccagaagaggatgctggattccttggaactggaattaaagttgggtgcaggcctccatgtgggtgctaggaacggaacccaggtcctcctagaagatcatcaagtgctcttaactactgacatttctctccagcctccctaactctcatttctagattagaagcattgttgatagagttttacagcccccactgcctttgggcaataggagtacctagctttcactgttcatgagaaccccaaatagaggaagacgggtaagaggccattctagcaggattaaggaagacctggtgatggtgatgcttcccaaaactgctcacagctcagctgtggggccactgaactcctgaggccaccaacagacccccatggccaccatgctaccagataccagatggctggtgactggtcacctacggccccttcccagagaatccttaattttaataagttcttcctttcagagattctaggaaagttaaaaaaaaaaaaaaaggtgggggaggggggctgggattcttaaaccaaaaatgtagaaggagctcttggctggttggcacttcctctatagaaacaaccagagctctccaacccagtgtctgaaaggggtaggactcattgcaggaagaaggaagccattaaaacaaggagtccaggaggagccaagtagggggaatgaggtctcagcagaaggtgggtactggggagcctcaccctggccgatgtgctccttccgcagttgtagctgagagctggtcatcatgatacggatcttgctgagctcctgggagttttggctcttcacacctgtctcttgctccattctctttgatgatgtggtagctgaggggttgaggaagcccctctagatgatggagcatttagatcagaccacatagctatctagcagtgacgtaaatggcatatccatggccactcttggtgtgctgaggggcaggaacgctgtgctgtatgcaggactgtgaggcatcaggttcagctctgcctcaccatcatccctcagaccaccgtcatccccgacatttgcaggctttgtacgcagactcccccccaagctgtgtcagacagccctgttctatctggctctgtgttttcctgcatggtttgtaatgccgtctttgatttatttcaggttccggaattgtgtttacacttacaggattctgcccaatgaggacgataaattcactgttcaggtaagtcccaaaccaacttgtgctttccctgggagaaaagaaaaaagagaaaggaaaaacaggtattctgaatagttgcaatgtgagaacctagtagagcttagctttgtcccccaaagctatgtgtctggggaactgagtgaataacagtgactcctcatagatgacagttataagatgatagtcatgcatgctaaggattattggtggccatggctgagaaaaggacacagtggtgaaggaaagccaagccagtgcctatgccttgatttattcttttgtttgacttccatggatgagcagatgtcagtggcccaggaaggagcagagggaggtgatcttgcaagagagatgggagattagatgagatggggcctttaaattcccatagctcccttctgcccaaagcattggtcatggcatctgaatcactgtaaaaaggccaaatatacaaagccataaataaacttgttgattgattgattgattgattgattgattgatgagttatgccacagtatgtgtgtgtagaggtcaaaggacaacttgaaggagttggttctctctttctgccatgtgggttctggggattgaactcaaatcctaaggcttgggtggttactgcctttacccactgaggcatctcaccagccacagctggattttaaaagggaaacaaacaaacaaacaaacaaacagacaaacaaacaaaaaatcatcagtgccaatcaaagccggggaagaccacccttgcagtgtttgtgtgacaaacacagggacttgtctgaccacagagtgtggcagctgagtgatgtggaagccagagaagctagtttttgcctgacttcattggggaagcatgggggcgtgcagaaaggaggtgacagcccccatgtcctcagtagaggagagggctgtgtgtcattaagctcacagcgaccatagtaagagtcacaacagcagatgggacatctgtgtgagcagtgcagtaaggaaagacaagcatctagggactgggtcaccagaacaacaaggagcaaagtcttcaggattttgctgaaaagaaaggggtgggaagggagatgtgaggcatgttggtggcttccggaatgtgacaggaatgatgaattgtttcgatcaaaggggtagactcagagctgccttgttgaaaggcagtccttaggcaaaaggccccagaaacatggaagcgagagagaagttatgaacagaggcaaagcctcggagggcagctcatgatggaattcaaaagaaaccacatcagtacttccaacagtcacttcctccagacggtgggactcgaagggggccagttccatccagggtgtggcaaagcttccgtctaggggaagggtctgctcatactgagcttcatgggagaggccagcgtgaaccactgtgggctggagtaacggctgtactgcatagcatggtggtcaggctgagcacagttcatactttttctcgtgtgcgcatgaacaccaggatggaaggcggggcggatgggaggcagggcggatggaaggtgtggtctaggggtcagggagtccctttcccagcccagtcattggtgggaagcaacagaacaaatctgagtacccgcaagcaagcgcccttttttaagcttggcaggtgttcgttgcctgaatactttctgagatgtggacgccaccagcttctggagacatggctcaggggaggtggcttaggcatgcagccgtggtctctactttttttttttttttttttttttctaagcctaggactgtgtgaactttgcctggtggcttggtgtccttggcaagtggcttcctgctaggagtcaactctcttaagttaaaagttagtcggcatctttgatctttctccccttttgtggtttcttctgttccttcaaagtgatgaagactttaagtatgactggaaaagaggtgactctgcggcttcttgttgagggtttgtgcccacagcttgtgaacccataactctgcctgtctggagtctgtacccacgttctcttcactcttgcttccgtggcttcctgtgaccccagaaatgaccacagaccttcagtgggactaggagcttcagtgtgctttgggagtccctctggctgatttttttttttttttttttgcaaaggtttagtgcctttggtggcctgaaggacactagagaagcccaggccttgggaccagaggctctggggccctggctgggttctacctcagtgagtcagctcttctctgtgggttgctcctcccctgctgacagcactgctcttctcagatcccagagggaacctgattggctcagccaccaacagctctctctgcctgaaccactcaccagctactgagctctttgctgcttggctgttaggaagcaggtacctgcccaccactgacagatgaggacagatcatagcctcctgtggctacctcagccagggcagtgagcaggcagtttttatagaagagatatggccttgctggacacaattggtttcagtcttgcacttgacctatgaccttcttggtcatctataccagccaagggacaggaagccatgggcctaaatggtttgaaagtccccacctataaggatgtcttgtcctataaaaaacaaacaagcaaacaaaacaacaacaacaaaatccccacacatttacaagtgctggagatgtagataagctcaattggtagagagcctatctagcatgtgcagacatggttcagttccctagtactgtgtaacacctggcatagtagcacatccctgtaatcttagcacttggggatagaggtatagagttaagaggatggaaagtcttcaggtacatagtgagttagagatcggcacacatgcatgtgcacacgctcatgcacacacacatgcatgcacatgtacagaacacacaccagacatgctaaaacaaacaatataaggaacaaatcaaagctcacagccagtgaaggcaaggcagaaacattcttcaggaagcgttggtatttggccactgcttattttaactccagttcctcttttcatgttgaagcaaaacctgcattctttcctgagcttagccacagtttctgtataactcagtccttatacctaaacctgcttagtgtcagcagcagcaggagcaaggctgagactgcacagcgacacaccaagggccttcatggtcccagctcttcttatgggaggtccttgttgaaacattcattccaccttcaatctttcccaaggacaatctgtatctcgtggtccagaaggctcactgatggagggaagcacgacccccaccactcagggactcagctctgacttcattacttatagacctacaaaatcagaggtgggaagggtggcagcaggattctcttgtttaacgcggtagacactagcgttcacaaccctgcttccaagctaagtcttctttctcagggctcagaacatgtttccaggcataaatgggtactgaaacacttcggcagtatgggacccaagaccatttcacggaggggaggcagggtagtggtctctctgagatgggcccttccttgcctatttggatggtatttgatcatgaatagcacttggctgtgtgtcctgtgcattcaggattttgcagccacatggctttgttcagggactgtgaagagaccaaacaggaagatagaatgttacatctcccctgctgtggtcacaattctgttcacgaatgacagggatggtctccatggaggccattgttctctttactgcccttgttcttagtaaggagacatccatgtccagttcttggtaagaagggagacagggggctggtgagatggctcaacggttaagagtgccgactgctcttccaaaggccctgagttcaaatcccagcaaccacatggtggctcacaaccatctgtaacgaaatctgatgccctcttctggagtgtctgaagacagctacagtgcacttacatataataaataaataaatatttaaaaaaaaaaaaaaaaaaagaagggagacagccacatgttcttcagtctgcaaggtgaaaaaagaatgggaagaaaatgggagggaaagagatgctgagaacagtataaaggggggggactgaaagacataaaagaaaaggaataatgagatgagggaaagataatgaaggaaggaacagtcaagaaagaggaaggctgtggacgtgatgggtacggtttctacaaaggaaaagaggtgggtgcatgcagggcgagaagctgcaaaggtgatggaggatgaagatggaagtgtgctgtgggctgtggaaggtgtctgtggaggatgtctatccctggctctgctccgggcactgaagcaaggatccacagacactgtgagacaggtttaacgcagattctcagacccagagagtgttctgatttaaagtccatgcggacatcctctgggtactgtgtgaggccagaggcaggcaggcaggatgcttacttagcccatcgggccccgggttcccatgcctagaggcggtgttcgtgtgaatcctgggtccttcctgaatctacaaaggaagtgactctcccctcagggaagtgacatccatcttgggccgctgacttcagagtgagctgttcacccagttgttgccttttggagtagctttgctccctgggaagccagggagggtctgaggcaggacaagaagtcttaacagacaactgtgttgtgtctgaggaggctgaagagctctgagcagcctttagacatgatacctctagccaggtccaccactgaagcgaataggggcctctgggcttctcttgctgcctgacacttcctatgaagaagaaaaaagcccagctcatgattctgtcgcataaatatctctgtgaacttcctgtctctgctgagccggcagtgttggggataaggactgtgaaaactggggtactgtgggttttcatgcacgttcagaagaagagaggctttgagatggtctttcccacagacgccaaggttactcttccagacagaaggaaaccccactagccagagcctgtcgtgatctgtgtagatcagacgcgtgactcaagttcctggtccctggtgatgtctgtcacattgctctgggcagttcggcatctggagaaggaacaaaaccacgttttcagagctggccggtcacaagcgtcggggagggcgaacggttgagccgttgttatgaagttcaggaagcgggtggcagctgttattgttctaagtggtcaccaagtgaaataaagattccgtctgagcctctgggcaaaggatgtggctcaaacacacatttcctgaggttgtacaaggaagaatgcagaacaggctcagaagctagcatggccaatgtgaagacagggagacccctttgtgtcctggcaaccatattctggggccctgtgttttaactttgctttattttattttattttatctgctgtagatataggtctattttctaagcctctgactatctcaggacatcttgcagctgggttcatatttaaaagggaaattcttggggccaagtttcccttcagcatcccccagaggtgtcgttccatagcctttgaaaatgtagtatgaataagtcccaagcaccaacccaaggccagctcgtgccagttgtagtagagagacaggtattaccttttgctcgctgcatcctttccttctgtggatcctgctgagattgatcaatttgcaacacaggtctttcaagactcaaaggtttttcttgtttgactttttttttttttttaagccagtttatctgttttatttttctcagattctactgagaaagcccagtcctgccctcatttgccagccttctggaacactcttactcccagaaacaaacaagcaaacaaacaagcacccctcacctgccccttctcctgtgcgcctggagggagagctaattgattctcaggtgtctgggggtaggggtgtttacgctctgccatctctgcctctaaatggcttatccttttctccccatctctgtgcacccaagccgtcttgttgctaattctccatatcccagaatgttatggtctgctttgtttgtgttttcttctcctgcatagcacagagctgacactaactgttgataggctaagcccagggaacagtactcaattgcagaggggaaggactccgtaggggcgtcttatcaaaaagtctttcttagaaacagacacaagacaaaatggctccctccctcccaactagaatatgatcattggaattgctgctgctgtttcacaaccgtgagaaaaggtgactggagaatgggaccatgcagacagcagagcagaaagataaaatgaagccaggtcttgaatggtgctattggattagctgcatcgaattaacaagtctaggagctggcctgcccatgggcttcctgttagtgagatgactcttttgtgttcaagtacatataggattctgaaacacgtagctgaaagtggtccaacatacaaggtttggtctatgctaggggggaatctcaaaaaaggaaaaccgtacacttcagattgagcaccgagagtcactgcacaggtccacaggctgtggtctgcaacctcgatcacacctctctccttagtcctggccagtgcatgctgccgggattggttctgtaccatccagacatccctaacgatttgtctctggactcaatatcctattgctctcactcaaatcagaaaaagtacaaaaatcaatcagcatgctagcgacctctgtgccctggaaagatgtctccatttctgtttctccatgggatgatttagtttctcatttcagctttgccctttgagagcaggtgtcattcagagttgtattgagcagatgagagtctttcctcggaggaaataccatttcccccagtggaaccttgctggatcactcataacatagccgagggcgtgggtaaggtggctcccaaggaaagtgctggccacacaaacatgaggatcagagtctagaaaaaagaagggctgcaatcccagtgcgggtggggggggggggggcagaaatagttggatcccgggagctccctggccggctaggctagcccaattggtaagctcttggctcagtgagaccctgtctcaaaaagtaagacggaagggctggagagattggtgatccagcagctgagagcactggctgctcttcctgatgaccagaacccacgtggtggctcacagccatctgtatttctaagttcaaaggctccagtaccctcttcttgttgccttgctagggagcaagctcacaaagcacatagacacatgcaggcaaaacgcccatccgcacttaaaaattcaaataaaagaaaatgtaagtggacgataactgaagtcaccctgcatttgcctctggcctctacatacgcatgcacacatgtgtatgcgcacacacacacacacacacacacacacacacacatgcacacacaagaacagactcctcaccctccacctgccaggtgattattcaattttatgccatggtccttatttattttttgaggttctgaaacccttctcagcccacagtgagaatccagtagctatcaatggattcggtctgcttgcatcttgagaagacagaagagctgcgggtcccactggacagcaggatttgctctgccctgctgcagttgtctccacagtgctgttactggaatccatgccttctggcacacgcttttctcccggtggtcaagttcttccgtgccctgagaagacttctctccccagtgactcactcttgctggaactacctcctgcatttcagtactgagattccaactatgccaccaagctaacctcagtgcctagtcactctctactgaccctgttaccagcagctctttcatctttccttcctgtgggttctctctcctgggtcctcacgacctcatggctcaaggctatggggtcctgtccaggtggttactcccgaagccatgttttcagagaaggctcttagccaggtcttcggatcctagcacccaggatgctgtgtgcgttctcttaaagatgccatctgtgcttctgaacctgtctcctcctcaggagtgctgacgcactgtggcatggctttcttacagaggtttaaggactgaaagaggtggccaaataagcacagagtatagcataccactgtaaccagtgcatgatggggcaaataaaccagaagtccagaggctggttagctgccacgccaacacagggtgatccaggcaaggctggggacaccagaggcagaaaagagcgagacagtggtgagacttaagaaccaccaaagtctctggagtttggaggaaggcagctggaatgcctaaggtggaacttgaaccaagattgagtatccagggggtcctgactggcttccaatcttggccacgaacaaagtgggagaccctagattctgtatctaggaaaaattgctggcacctgctcactggggcggagtccttctactttggaagaaccaggaaggcagaggcaggggatagggtcatggctggtacctggaccactggccatgtctaagagagacctgaggacataggggtcagagagggtcccatgacttcacctagggtcttgttgccattgagtctgtttagtgagcctccatggaggcagggacttgggatacactatttcatttaatcctgggaagaggaatggtactctaatggaaacaagaccagtgagatggctctccagacccacctgtggctgaccccgccacccccaatgtcgctttcagagcctcttttccaggcctccccatgggaaaccatgtttgcccaacacccctctccactgtaccctgttctgttttcctaacatcccttagcactatctacaactagttgccagtagcaacataacccagtagtatacagtcatacacattgattatcttgtatttttggatgccagaaatccaaaaatggtcttcactggctgtgctaaaatccagatatgagcagagctctgcaccatttctgaagacttcagaaggaaatcttcctctgtccttcaagccagcaatggtggtcaatatgtctcacagtgccaacagaatggccctgtgattatcctgattaattggtatccttttgcatctcagagcagaacatgagcaagcctagctcactcatgctggatgacagcttattcacaggttctgagatggggacatgaacatctatagaaccggatggggaaatcattattctgcctcccacagcagctatgttcatgacctatccattcagctatcctacctatctccatcctgagaccagaagatggatccaagagaactgagaccttgcctgtcttgtactgaagtgccagaaagccacctacactcattaggtactcaataagtagtgagcaaatgcaggctctgcattaggtactcagtgcagataatgtaaatactagatatatccaggcacccattaggtactcaataatacttgatacatccagataccagttaggttctcaataaatactggatacatgaaaaggaaggagtattttttgtgtttccaaatagccctgtccatgctatttgccaaggtgcctttaggacctctctgcacaggcggatttctgagttcgaggccagcctggtctacaaagtgagttccaggacagccaagactatacagagaaaccctgtctcgaaaaaccaaaaaaaaaaaaaaaaaaaaaaaaaaaataggacctctctgcagcagccagtaagactcgaatggcctgggatgatgccagggtcgtggagcatctggagcaaggctaatcagacagttgccctgggctgccccttgaggcagctcctctctgggctggctcccagggccaagcacatccacttgtctgcctgctgtcactcctgtctcccagagtaacttgagttttacggaaagtaaagagagaagctagaagggagggccactcggggatgtaattaaggataaaaatatgccatgtcctgggctctggcaacatggaaatctagattgctcatgttcccaggactgtgttgtctgtatccaagtggtcccaaatgctgtgtctaaattcttagcagggaccatttaaaagttatgtgtgtgtctgcctccttccttttctttttcttgggatgccaagaattcaacccaaggccttacataccctaagtgagtagtactctaccactgacctacaccccaatgccccaagagtacatctaaagacagattttgcttgttcgaagtggggagggggaaatgcatatggtaagaagatgaacttccccggttgtgtgtacgaggagaaggcttccttaaagttagctggcaaacatgcaagtaggagaaggctgatatttcctgtcaccaacatcttctgtccagaaacacagcctaaccaataagcccagctttcactatctccagtcctgaaggacacctcgccctcactgcctgcccagggctcagtgattctcttaagataaaggaagagtgggtggaataatcttgacctcaagatggaaatgattgcacaggaagagcaaggggtggggagattaatgtggcctgcgaaaaaaaaaacggagaaagcaagctgagggatccatgtgggcttttgtgacacccgggaccactgcacactatagtcttgctatagatgctatatagtcctgctatatagatgtggctaccatctgagaggtgctgagtcaacatgtgctgtctctgcttttttttttctttaaggcaagtagaaagcaggaatcaaaaataccaaacacaagtaaaatcccctcacctggaggccagtttctcttcctgggaaagtatcagtgtgaatacacttctctcaaatgcagctgtagaatctgtaatgtctttggtaccttaaaaatggcgttgaagcatggtgtgggctctcagaggagttctttgaaggttcatggaggagagaactgcaaaaaacttggcaacaggggctggggcagtggtgtggtccccagaaagcacataatggtgcttggttggtatgaatatgattaaattcccagcactgggtggaagagggacaggaggagccctggagcttacccagcctaacaaaatattctttaaaaaaattcactctgacaagaggtctggctgcttgactcggggataaaggcaccttccacactatccttaccacctgagttttgatctctaggatccacataaaggtggaaagagagaaccactccacaaagctattctctaacctacatacatcctacacacacacacacacacacacacacacacacacacacacacacgctttctctagtaagaaagagacctacatgaaggaatattcatttttattgtacatttggtcaattcacctcgcctaaaagctcatcaagtgagcctcccccacgatatctcttagctcccaccaagtcatctttgtctcagtatctgtaagtttcctcaagcaatggataaaatttgcatcctcagtattttggtttgcatttcctttattaaaaataacaataatcccttttcacatatgttttcatcatttgcattccttttcttataatttgcttatttatttatttttcgtttgttcatttgtttgagatagagtctcatttgtctcaggttggctctgatctttctgcttctgcctccagtgctggaataacaggcgtgtttctcccatgctcagatttaatatttttatttaaaaaaaaaatcttttggggcgggagaaatgtctcagcagctagaagtacttgttgctctttcagaggatgtatgtttgattcccagcacccctgtggcagcttataatcatccatttcaggggacccactaggcacatatgtagtgcacatacatttgtgcaaataggacatccaaacacataaaataaacaaatctaaaaaacctttctaaataaaaatatgttcttgccaggtaatggtggtacatgcctttcatcccagcactcaggaaggaaaggcaggtagaactctgagttcaaagccagcctggtctacagagtgagttccaggacaggcaaggctacacaaagaaaccctacctctgacctttgtcctcccaatatacatatatttagtaaactaatttttatttgattagtttactttttcttttaaagggttttccgtgtgtgtgcgggagcgtgtgcgtgtgggtgtgttgttattgttgttatatatgtgtgtggtatgtgtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccaccaccaccaccaccacagccaccacagccaccaccctgctgacaaggctcgggtcacatgcacacaccactatgccctccctgctctctatgtagttctggggatctgaactcagaaaatttataaactaaatcatctctctagcctgtctttttttccagattatattattatttcacaagtctatcctgacttgtgaaattttttgctttgttttgttttgtttttcgagacagggtttctctgtatagccctggctgtcctggacctcactttgtagaccaggctgacctcgaactcagaaatctgcctgcctctgccttccgagtgctgggattaaaggcgtgcaccaccacgcccggccctgaaatttttaaacagaggctgggatatagctcagttggtacttgcctaatgcttgctagaacacagggagccggccctgtgatcagtcctcagcatcataaaagcagcatggtagcacacacctggaatcccagtacttgggaggcagggctgggacacacagaagatcagggtcattttcagctaaatagggaatttgcagccagcctgtgcaacacgggcttctgttataaaacaacaacaaaatcattttagcttatcagtttttctcctgtggattctagatcttgtgactcaccaaccttaattattggggggaaaatagtattttttattttctaacacttttatcatgtttctatgtcaagtgctttattttgacatttgctttaaaatggggactacaactcttcttcccggtgactcagcagcctccgatagtaaacagtcccccctttcctgccgactcacggttcctcagcccatatgctaacgtcccacttcctctcagatgctggatgtagactcctcctcctgttccacttattgattagtctctacttgcaataatgcagcgatgcacattggtagcagtgacattcttatggcctgtttcggatttctgctccagaaggagactataaatcaagaccagggcagcaaaggggaagaggaagtgggtaaataagatgggggtcagcataggtcctgtatgaaggttccagccagtccactggggacttcttcttaggtaaagctgaatgctaggtgagtggtcagaaccagacaccaggaactctcaggggataacctgtgcatcccacaatatgctccttaaaggagaaagacatctgactcctaggaccattcctaccccagctggttcttcctctcacaaggagagttctagccaccattctgcatgtccaccctaacctatgatgtactctccctttcccaaaggaccttaggagcccagtgctgcagagttcacttcctctcccattcagcactctcctaagagctcagcttgacccaccctacctctgggaagggaaggcccttttatttacactgcgcttactcttatcttccttgtgtgcactttttcctacctcgatgcagagccccagcttggttttctcataaacttcccattgtctggtctagcatattcagatcacccagggtctgcttgtgcttctgtcacccagaagggtggagatgcctcctagctgactggcaaccctgtaagcccacgtctggtgtgaacagctgatggcatggagagacgtatgtctgttctgacttgactgggatctgactgagctggtcttttttctgggtggtcctcaacagctgatgaatgaatctcacaagatgctttgcccaaatctacatagatgagcttgatggtggcaggcaggtggctgctgtgatgttgtagccatcaaggcttagcactgttggttatctgacacatggttttctttaagtatggaccagtgagaaacagttcttaaaattaacagattttttttttccacataattatttgaagttcttagctcctttgcagggagagggggggggggctccaatgagcttgggcttgttccctccttgcctaccatctcaccccaggactgttctgcttctgatacagaaaaggatcttcttatgggggaagggggagggacttgtgcggtcccatcaccccctgaagatctgtcggtggttaacaataccaggggaggcagtcagtttctccagtggtgtagctactggccaggcatccatgctcctgtcaataacctctcatctatgctctcttatgaacaacccaaactaaactcatcggcatatgtgtgtgtgcatgcatgcatgcgcttgctctcatgtgcacacgcgtgcgcgaacccacactgcataaatgagagataaaagaggggattagaaacagaaagagatcagcaggtatgtgagggggacaacagagggtcataggaggtaaatgtgaacaaaatgtgtgtgtgtgtgtgtgtgtgtgtgaaaacctcacaataaagctgttttttctgtataattaatatatataaatctaaaagatttaataaagagcctaatccttaaaaccatgtttcactgactggattggtccccattgcacacccactgtcccctagaccgctacatgctttgcattttctcatagaccctgctttggcagattgccccgacctgaaaccttacccaagacgcacataaccccaccaaactgtgaactgcagagtaagccttctcacccattgtgaaagtcacaacttccccgatctgcccttccctttaggcctcccctcccccaaggcccagttctcttgctcagaggtctcttccttccctgggatggagcttgggcgtcacagcagcaggtgtaactgcatctgtttgcctgccttggatctcaggctagactgtgtcttgctcagttccttggagcagcaagaggcgtcagaggtaactttcggttcctctaaaggctggtgtttatcaaagtctacctaactgaacttctggcctccagaggtctggggcgtgccatgctgaggagctgatggacccatgctatcatgacagcttgccaagcagagaacactttgttcttgtccctttccggctcttctcagcctcagacaatcccaccacccaccacggcaagctggaaacttcctgttttggtctgggcctttgccatctttaagttggaaaatctttgtccttgatgaatgctccttgccctctgcattgccccagaagttattaacctttttgtgaccactgtaatttcttcaggattcaatgttgccttctaatcctggggtcatgcagaaggaaaaccaggtatagacaggaagcccagtccgtaggagatgatgctgttagaatggctgatgtcactccctatgacacatgaggtgacaaacatctgagagctattctcctataaggaaaacaatgcccccaatcttctgccagtgtctgtggtgtcaatattcccaataagacgcccactagaatggctgcttgggattgaagtcagtggaaccatgggtaataaccctggacagtgtgatataaactcaaattggaagagctgacaaagtcctgactctagactttgagcatcttgatcagagcctcattttcctagttggggtttctgttgtcatggagaaacaccattaccaaaagcaagttgaggaggaaaggacttatttggcttacacttccacatcacagttcctcattgaaggaagccaggacaggaacttcaacaggacaggagcctggagacaggagctgatgcagaggccacggaggtgctgcttactggcttgtttctcatggcttgctcagtctgcttttcttatagaacccaggaccaccagcccagggatgaccccacccacaatgggcttggctcttcccccatcaaccactaatcgagaaaatgccttataggattgcttacagcctgattttatggagtcatgttgtagacaacatttaatctcagtctgggtttctaccctgtctttgatcattcaattcccagataaaagacacacaacatgattatttataataagcttttaatgcactagagctgggtagatatctaccctctaaaccatctgaatctacttccctacccataaccccgagttgtcacttgccatgttccatctgggccactcttaactccaagtggccagcatggccatgtttctataattcacctaccccatggtaacttctccacattccatcttctctctttcctctcgtggttttcctccaagcctgggaactcccaatccctgcctgtctcaattctgcccagctatagactgtaggcatctttattcaccaatcagggataacttgggggaggagacaaggttacagagctcttgggtctatgtgcagattctcttgtccctgggggcaaccaggccttggggaccagtatttagcattattatacctagcaaaagaccaaacctccacagaggcattttctcaattgaagttccctcctctccaatgactctaatcttgtgccaagttaacttaaaaatagcccacatacccctgtacctcagaggggtcaggcaagttcacagtcacttccacctgacttccggtcttttcaggccctaaacccttactcagagtcattttaattcagtattttcttttctttttatcacctttggggagggatttttttttttcccaagacctcttttacagacttgactgtgtgatcaaacttcccatttctctgagcttcggtttatgcctctgaaaagtggaaggcaactgcccttagcagatgacttaccccgggcttccagcagcaactaactctgctctcggccaagcagatcgtataatcctcttccacagacaaggagagtagtgtagcctgagagcgttaagtatcacagcgttcacactccaacagcaaaatgccccaattagaagcaacttaagggaagtaaggtttacttggggtcatggttgagaggatatggtccatcatggtgggcaagcaggatagtgggaccacaaggcttgccttactgtatccacagtcaggaagctgaaggagatggacacctgtgttcagctgcctttctcctctttcattttttttttttttatagagtctagacctcgccccatgagaaatgctgctacctgcattcagatggtaaatctccctcagttaatcttctctggaaattccttgacaaacctgttgagaagtttgtctagtaggggattctaagtcctgttaatgtggcaatgaagattaactacaaaacagtagagcctcgaattaacttctcttgacatttgacatttgtaaattctagcaatgctaggtctatgttccccacaggctataattagaactaaatggaaacaacttcaacagtagcaactcctcctccttctccttctccttctccttctccttctccttctccttctccttctccttctccttctccttctccttctccttctccttcttcttcttcttcttcttcttcttcttcttcttcttcttcttcttcttcttcttcttcttcttctcctcctcctcctcctcctcctcctcctcctcccgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactttaaatttgtaactacttatttgacagggtctcattctgtaaccagccctaaattttttacaactttctacaaatggtgcttttggcctgggtataagcactcaaaccatgaatgggaacattgtatatttaaactgtagcaaccctctgaggctttggttttcaagttgtcagtttgagagccctgacatgggcaagctaacagaataggactgtgcccagaaaaatgagtgctagcccactgcccctaccttcatctccattgggaaggaagatctggggtctccacacaggctgctccaaaaccccacccaaactaccttctcagcccaacaaccttgaattttccatcaccttcttcacctccatccactcacacagaacgaccggacactctgtgactgtgtttatgttctcagtgtatccaaattattacagctgagcactgcagatgacccaaggcagctgcaggttttatacagaaacctctaaaagtctgtaaacctgggatggttttatatgatctttataaatttgacacttttaaagaagatctctttgaaaaacctttccctccatgtacatgtgtatgtgcatgtgtgcacacacttgcacgtttacatgctaaaaaaatgatctgcattacataaactgattctaataatgcttttcataacccatgttgaaataatttttttgcatgtaaacaactatgatataggccatttccagcctggtctttttttttaaataaataaataatttattttgaaagtaaattgacttaggaaaaattttaaaatagtacaaagaattgcatttttctctctcatcttcccactgagcgttttgacgtataatacactgatcattctctctcatattgcactgctgtttttctgaattattggagaggaagggtctgacagtaccctattatcactatctcattcatagccatattcagccatcaaaattaggacattaatgctggcctaccgtccactcccatagctccactcaaatgcctccagcagtcttagcagcaatataaaacatcctatttgtgtaactatgtgaactataatttatacacataaaataactttcctcctggaccaatgtttagtcgccaaacctgggggtgctgttgtgttgtgccttccttcatgatattgacttttttttttgtttttgagaacttggtagtaattttgtaggctgcttcttggtttgagttcttcagggctttcctcgtggttgggtccagttctgcatttccatttccagcaggacttctcagtggtggtgctaaacttactactcctgctcaaggtgacatgccggctgctctgcgcagttgttagagtacgctctgagtgtgaagctaagataacatctgaaggggtttctctttgcaaaattagatatttgttatttggggtcttaatattccctccctctctccctccgtccctccgtccctctctccctctgcttaggattgaaaccagagtctcacactattctgttcttggccactgatctatcttcacaccctcaggaattcagggtcgtcccaggcatcatagtaagtccaaggccagcttagaccacacgagtccctgtcaaaaagaaaataaatgactgcagtcaaggatagcagcacagcttgaaatcccagcactcagaagcacaggcaagaggatcaggggtttgagtccagcttcggctgcataataaaaccctgtctcaaaataaacaaataaaagactgatgactaggctgatagctataaacaacttgaatgtcaactgcctagcatacccaaggctctgaattcaacccctaacaaagcaaataaaataaaactagaaaattatacacacacacctgttttgtggaaataatccgagactatctaaatgtgtcatctctcaaactctcacccacctggtcttagcatcccctggtgaagtgcgtctgcatcagtagttaccatgatagctgccaaatgctgactgcctcttctatcattggtcccacagttattaattgacatttcgctgtagggaaagcttcccttctgatttatttatgtaaggataaagttgtgaattctcattttacccagtggattgtaactatcttcattattcatttcaatgttcagagtgtactggctgctcctcctgctacacttggtggttcctggctctttttgacaaattgtcttcccgttcaaagcatggcttttctccctagaacctactagtgcagtggccctttaatatagttcttcatgctgtggtgacccccaatcataaaactaatttcattgctacttcattaactataattttgctactgttatacatcagaaggtaaatatttttgaagatagaggtttgccaaaggggtcacaaccaatagtttgagaacctctactctagaaatatatatctcaggttctgctgtgccttcccagccccagattgtaggctcttagctgaatttaagaacctctgggtctttgtaatggggaaagcacccaagatgtgaacagtgctcattgctgttagtctctgccctctcagcagacagaaccaagaaggtgggggaggaagtgtgtgtgtgtgtgtgtgtgtgtgtgtgtgtgtgtgtgtgtgtatacaattgatactgttacttaccattcaaatgccaaacaacagaattgctctgcaactccctccctagcctggcttgcatgcttatgcttgttccttagcaggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccatgaccccagctcggaagttggctctagctacttcaaaggcgacgaaacaatgaaactgtgctaataaccagaggaaagggagctgggcatggtgggtcatgcctataatcccagtacttgggaggctgaggcaggaggatctcaagttccactctagcctgagctatatggttccagagcaaaatcctttctcagaggaagaaggaagggggtgagctgtcttctttggggatatcctttagagaaaccactctcagccctagaactaaagacaggtttctgagtctaaagcagtcccccaaactattctcagcccaagctgcccctttccccaggcttctgggtctctctagctggccacgcctcctcagtctattagtgttctagctgtaccagatccctgtccctgcccctttcccctttcttccaccctacctccctcattcaagtacagactcttaccctgtactgctgccaaagtgatgtcatcctgaccagcagtggtttgcacctgtaatcgccacactcagatgatgagtttgagtcaagtctgggctataatgtaagaacttgaatcaatcaataaataaaagttaaaatatcaatcctacatgaatgcacaccccagctcttgagtgctcccaaaagctttcaaaattattgttaaaaataatagtaagttctacaaaatcccagaggccccagaaatcatgcgctctccccttcacccacatgaagtctttttggccaaggcagaggaggtgagtagacattgagaagaaggcagacggaaggtagaggaaacttcctgatgactgcagagataggagagggcagaggggcctgtggagagagactcaggagaagctgggtcctagacacactgataactgcatagggagatggcagaccatgctatctaaaacacaaaggcctggtcaggccagctgaactcctgagctgcccctgctgccaccccccttccacagtcccccttccccttcccctgccccctgcccagcacagctctcagatcttctgtgtaagttgtatctgtggaacacccccaagcttggactgagtctagcccctgcctgtccctcaatcaccgtcttttcaactggcagactcagggtcctcactaagaagcagaaataataaacacgatcactggcacccctctgcccgcagacaaatgtgttttcaccctgctttcaccctgctgcctaagccttgtcttctgactttctccttggacctgaagagggtgctttgatttggtgtgaatctgcatggagctctttggtttgggttccccacttcccacttcccaccatctataaaatctccatggtaactgaatgggccggcctcagaatggctaagtagctaaagatcgccttgaatccctgatcctcggtctctgcctcccaagggctggcatcttgggcctgcagtcctgctctgggaagtcttttcaacctaaaaggaaggctaccccaaccttcaggagtggccacccggagcaccacacttctgctcagcggctggcagacttcctcccctgtgcccagtctatttcagttctgcatttagagatgtcagacagtgctgacattaaaactatcccctgggatgtttcagcttgtctagggctctgaccgcccaggcctgctctttcctgctccccacccccaccctgcccccagggtattttcagtcatccccaggactgcatctgaaaaagcagccagcagtctgaccaccagctgaccttcagatgaccttagtcttaaaggtaccagtgccctccctcctcttcctcaaacctggacccccccagcctgaaggccaaccaaccctccatacccaaaaacctttgtgcatggctttgtaccttctgggatattgctccctggtagatgcagatgccaagagatagaacaagattttccctataagatctagctgggcagtccactcccctccacctccctgcaccttttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttttatggagcagatgtatctcagaacagtgggctgtggttatgtaccgtaccagtttcccctgtcaacagacctggctataagacagggtttttcaaggctcttgtgaagggggggggttggtaaagtaggaaccacagttatatcccaaggcagagtgagctgtgcattcaaagcttgactccttgggttgttgtctataaaggccttataaaaaacattgcttctagttactctgtgtagacatattaactaatcagagacaaggactatatttcatttcgtgaacagaactaatccttactttattgtcttgtgtgtgacttttgattctgaacataataaattcatgtccagtctgttgattaagcaaatcccatggagaggtctatcagcctcaagcctgggatatcccagttacaagcattattttaaagtaggaagagcctgaggctgagggtggagcacagtgaaagggctcgggccacaccatgcaccacacccgctcatcttcaggttctctctctcttggatcaaactgcctgccacatgctatagttaatctgttaaggagagaaggaattcatgcctgccaagtggaagaggagagtcaccggggagcaggggcctcacggtccatgggtattgtggggtatcattcctaaggggcacagtgagggtacatcagtaaacagctcagaaggctgttagaggcccacagctaactcgagacctttggggttccttctctatttcaaaatgacttggtgtatcttttaaggaaggccttagctgcagaatgaggaacaaaagctggcggcctagacgctaagaatccagagccacgcccctctgctggcagggaactatgaactaggccttgatctgagcctcatgttccatgtgacacacaggtgcttctctgccatcatgattgtcctctggttcattcactcctttgagtttagccagtgagagccccagccaggtggtaagacagcgagaggaagatagtgaagaagcgggggtgggtcctctggcactggctccctcctgctgggagttctcagtgggctccagcactatccttctgctgggaggttccaggggggtaggggtaaactgcttccctccgctgttagctctagaactcttcctctctacctaccaaactctccccaaatgccagttttatatggccatctgcttcctgctaggactatgactaatgcctatgacacctttcttgaccacattttacattttaaaatagtcgaaaaagcaccacctttgggattgttgggagaaaagggggtacaccaagccccacctctcactgtttctacctccaagcccaagcacacagaaggtatctaacctagttttatttcctgttgctgtaataacaaaaagcaactaagggggtggcgggaggaaaggtttgtttttgttttcactcataattccacattacggccctttgtaacggggaggtgaaagtggtagccaggcagaggtggcgggtgctgttggtcccagtgcttgagagagacagaggcaggtaaatctctgagttcaaggttagcatggtctacaaagtgagttcaaggacagccagggctacacagagaaaccctatcgtaaaaaaaccaaaacaaaacaaaaaggcaatactcctctcctattccctgcccccccacacacacacacgccataggccaacctgatctagacaattcctcactgagactctcttcccagataattctagactgtgtcaagttcacaattaaaactacaaattcttgcctgtcattctgattccagtgtggtaaccccacagaatcaggggttgccaggctcctcagaattacaggaagtacggtagaccgagatgtagtatgtagaccccgctcctgtggggaggggggcctcttaggcaggtgaatatgtgcaacatctcatgtggaaggggacctctaggctcattactaaaggacattattgttcctcctggcccacagtcagtacactgccacctagaagatcttcccacgtgttggtttaggcttagtggggtttattggctacacagagccggccattcactatccactctgcctggacatagggccttttaagcccatcttatgaagacagcattttatactgtgcctccaagagacccctttctctggcctgttgggacccatatctccaccatctcctctgcctctgtctgctgcctgtgaagacaggctgcttcctgctggcccacatggcataacaattgaaatttatctcatgggaatttgcttcccttaggagggcccttgtatcctttctgggtactagaagtacaaagcactcagagctgtccctgagccccagctcctgggtagtgaatctatattatagtgtggccataaggcctttgcaaggacatttctctgtcaaatggacccagcacccacctggcatctctgcctcctgtctcatccccatctgtctcatcagttggccttgtatatgtgcatattcattgcatacataaataaataagtggatcttgttgctctgactgagtaccacaaacagggtcacttttaaagattaaagtccaggcatgtagcttgaaggtaaagcaaatgcctagcatgagcaaggtcctagccagacacccgtgaatagaggcatacagcttatttggctcacagttctggatgtaggatcctaggagcaatgtgctgacttctatctagggaggaccttcttgctgtatcataacatggcatggggtatcatatggccagacagcaagggtgagctgagattgcttttcctctgcaacaagagccactgacaccatcacgtaaacacacacacacacacacacacacacacacacacacctattacaaaggctttacctccaaacaccatgtacatatgaatacagggattgcctttctaataagtgggaagacagtccaaactatcccacacttggggagttgtgcctatgggaggcagaggaaagcctcccttcttagcagcgccgctccaagctggctcatgctcatgcatccacctggaggagcctgcttccccatccttgacatttgacctcaaccctacactccccatcttcccggaagcctgacccacctaggtggtcagccatcctggcctgccaggccctgctccctggcctccatgacaatgcaagcatgagcaggggccagggaggcatggctgactaacagctctcagcagtcacaactgcctccagctcctgcctgctccttttggggatccctttagcttctccttcctctgcttgcttaatacacagcctattcacccaccattgtttaccccatccccactctgcctgccacggccaccttgaacatgtctacaattcaatagtgcagcatagagcctcgtctctgggtttggttctggctttttttttttcctgctgtataagataatgtgcatctcagcctgcacgccccctaagccccaatcgcaccagtgttcacacatgcagtccttaggcactctccactccctgatgagactccttccccagattcactcaggagtcagcccatctcaaatttctttgatattttcattctctttccgaatgcccagtggcattcgaatcgattcgaatcatcgattctctctaaccagccctgacaagaccacacacactttacctgaagtgagttcacaggtcttacatcttatcaaattattcttgttattccattcttgtttgcatttgaaccactcactggagaatattttggaaatcttggcaggggctgccactcaggtggtttaacagtgctcacaagagattgagcagcgtggggttttgaggagacaggggagtttattgaaaagaataaacagatgggtttgctaagctctctataaatttactaagacaacctaagccatacagcatgagtgtgtatatatgctctctcacgctctctctctctctttgtcacacacacacacacacacatacacacacacacgcacacgcacggatttttgtgtgtaaaacacatgagcacacaagtacacttggagagcgggaagcacgcagagggaggcctggagcccatatggacagcccgagtccacagagactgttctcaccaaaggccgggtgactcactacagtgtagaagacccagaacccaaggttcagagacaagtctgagagcttcctagttgtagcagctatttgtctcattggtgtaacaaaataccttacaaaagcaacttgaggaagaaacgagttaatttggcttacagttcaggggatacagtctacagcaatggggatggcacaggacgggaagcagaggccactggccacaccatgtccagagtcaggaaaccaggacgaacgctggagtcagtttgttttctcctctttactcagtcctggacctcagaccatgggatgatgtcacctacatttagggtgggttttcccacctcagtaagccccacctagagaagcccaaagacttgtatctatggtgagccttaattccatcaaattggcaagattcctgagtctgtcccattcacctgagagcataccaaccctcccaaatcaagcagtcaaaccatcaggaataggaatctaatatagatgtgactgagtcaaccacctgccttccctcctgtgaggactttctaactcaaacccatgctcctattggtctggtttaccccccaagcaatgggtttctttcagcaagaaggtagctagctggccctaaagaaatggttcagcctctgatctctaaacctgaagatggacacaagtcatgttaaaatgattggggcagctggatgtggcagctcatgcctgtaatcccaacacttagggagctgagacaggaggcttgccatgagtttgaggccaacctgagctactatgtagaacattgtcttaaaataaaataagttaaataatagttagacaagagtgaactatgggcagacacttggcccatgtggagggatcatgaccccacaaagccgcaggagactcactagagagagtatgtatctcattagctgcagcaatttggtctcttgctctggtctggctgtctgtagggatagccatcttagcaagggtatatctaaatggatgaaggggcatcttggtggggtctgggctaactattcagggaagaccttgaatttcctactggagctgcctcttccaccaacatacaaaagagtcctggtcccagagtgtgagagcagtagagcagagtcacctccatcatcaaaagcagaggcacaaactggtgatgatccctgtgttgcttggacaactttctgacttttggctgaaaaaaaaaaaaaaaacaaacaaacaagaaacatagttaagggctggatgggggaccaagtggttaagagccttgactgctcttgcagaggatcctgggttcagttcccagccccacatggtggttcacaacgatctctaactccagtctaaaggatatgatgccctcttgtggcctccatgggcactgcagtcacatagtacacaaaacaattttatattttaaaaaagtaactaagtctagcgtggttactagtgcacaccttcaatcccaggactcaagagacaggaacagaaaaatctctgtgagttccaggttagccagggttccatagtgagacccactcaaaaaaacaaacaaaaacatagaatttaaacaaatggaaaaaaaagtacaagcatgaattttcctatgtttaaaagcaataatcaagtgtggtggcacaagcttgtaatctttaagcccttgaggatctaatgcaggagggtggttgcaagcttgaggctacgttgaattatatagccagaccccatttcaaaaagccttaactaaatacagtaaacaagccacgtgtggtagcacgtgttcttcctcggagggttgagccaggaggatgacatattcaagcttggtctggatagtttagaggaaacctgcttcaaaatcaagtttaaaacggcagactgttgatgcctggtgacacacatctgtcatcccagcacccaggaggcagaggcaggagatgtaggacggatgtttccttagcttgtgtacagtcctcgcttcaatccccggtgctgtaaaacataagtcattaataaacaaatagtcggggcccggggtgtcactcacggtagggctatgtgtgctttgcatgtgtgagaatctgagctctaccttcaataccaaaaataaaatataatttttagaaatggtcttggtgggggggggagcaaaaatagctctgttcaccacacacacacctaaacttgtaaaaatagaactcttggccctttgagattataggtgatttattttttttaaaaaactggtattttctaatttggatataataaaaaaatatatactaatttgtatagtaaagtacttatagaaatcatttttaacagatggagagaaaaaaatagttttaaaaatacttctatttgcacctttgcaaagggctcctgggagccactggctgcgggatgtgaggcgtgagtggagagcttttctgggatatggtcactcctagtctcttcagtctctgctccaggtggcctttgtgcttgggtgacattctagctaaagtgtgctctgtccggggcttctgtcctcaggcatccgaaggtgtccccatgaggttcttcacgaagctggaccagctcatcgacttttacaagaaggaaaacatggggctggtgacccacctgcagtaccccgtgcccctggaggaggaggatgctattgatgaggctgaggaggacactggtaggaagggaaggaagggatggcaagtgtggggaggtgacaaaaggactctgtggtccagcctcacgggtgcttctagactagagtacctctttggaggttgaaggatgctttcacaggggtcccgtaccagatatctgcattgaaattcataacagtagcaaaattacagttacaaagtagcaacaaaataatcttatggttgcggttactgcaacctgaggaccgtattaaagggttgcagcactggattagacagtcttgctggcacagaaatgattggccgggaactcatatgactctgaagtcaggtgtagactagaattcctgctccccggcttgttgcctttgtggccttagggacttctgactctctgaaccacaatcttgcctttaaagagattagataagagtagcccatattaatctctcaggatgtcacaggaccagatgagcgcgtgaatggaacaccatcagcagtgcactccagaaatgaagttgccatcagtttcttcactatagtgaaagttgagaggctccatgcacataagtgagagggtggggaagtgcttcttcatgtaccatcactaaaggcaaccaaaatggtcatggtgggacatgccacaggctattccaaaatttaggatgccaaggcaggaaaagcatgagtttgagctagcctgggctccatagtgagacaccaccatctgtaaagaggcttctaaaatatgtcggagaaaaagagtttccaagagtttgtgaaggccagcacagagacaaggagccagtcttggagagacaggtatgcagaaagccctgtagcaggacacccttcctttcatctcaaaggttccttgcctgacagaagcccacatgttacctcgtgagacgctcagcaggtcagtgagttcccggcaggttccccggggaaagcattagacatgtagtgttgacatcccaggcctggctcctacctgtccctggagagaatatgccaggtgtcttgttatactgtgggctagctgtggattgcctggcctctgtttcaaccttctccttcctgaagaccctccagagggcagccaggagagctgcctcaccatcctgagggagccacagaggcacatggtgaagcatgtgtcttagatggccatggtttgaagcctgcttagatgggggactgccctgtctcctctgcctgacctctagagatcagcagctgaggtatagctgagaccaaaattcacaagaaggccacaaggccacatggcagccttgacctggttctgtagctatgtccttgacagggcaccactctctcttccctcaggctaccatgctttttctcagcttgctctgtcactggcatcccattagaggcaaacccttcaatgtgtcctataaaggctctggagagaatctgtttcctgaccttgacttcacctccccaaagctcctggtcacctgcaagacccaaagccaagggacacccagtgcctggaccaaggcatctggtccatcttgggtcttctcgagggcctgcagttccagggctcaaaataggttcagatctgtctcccacagccagagggaaaggaatgtacaaatacactgaagaaaaaaacccttgtttctgtcacctgtgtagtgctatggctgaccagagtggccatcagggggcagggcagggcttgggcaggaagtcctgagtctaaatgtcccagtcaccttgactttgtgcacatgaaggacatttgaaagggtgggcaagggttacaatctcccagatgaaagtcaagttgtttgatattttaggtctcagaagccctttgctttctaaatttgtttctgagggtctagaagagggaaaggaagcctgaggctggagagactgaagagaaggaaggcggaagtgagagacagacagacagacagacagacattgggtgggcttgattttctgcccctagtccctgactatactttctagaccccgagagccaagtcatcttgctgatctgccaatcaatctgtccaaggccagctatcttgaaggtgtccctgtgcttctcagaaccaactgaccctaggaggtacttgctgagcagcatggtagccagaacaacaagaccacagccaggggcttcagatctaagggtctgaaagcgaggtcacctcaagaggctccctatgtagtcagcatggatttaggcaggaagaatgggaatagtggggcagactgggacctggaactggggagcctggacctctccggcttgtggtagaaatgcggaacagtgctgtacggacagcatcctcactgatggcatttgtccctgctgttcttttagtagaaagtgtcatgtcaccacctgagctgcctcccagaaacattcctatgtctgccgggcccagcgaggccaaggaccttcctcttgcaacagagaacccccgagcccctgaggtcacccggctgagtctctccgagacactgtttcagcgtctacagagcatggataccagtgggtgagtttctacttggagggtcccctgggaagccggcctatgcctaggacccagcatctagcgtccgggcatgctcaggcctggactgcggggagcagcagacctgcttattcctagtaaagttgaaagccaatgtctgtctttctagtcaggactatctccctgcccccaagtcatgtttgttcttagtgcatttcgctattaagatcgtcacatttttcttgtgagattttatgatcttcttggagtatattttacattcataacttttaggtctttctgccctgtaagtacaagattgcttttttgtgtagaaggtcactgccgttctgtggtcagatggcacctccacacccacggtcgcccacgttcctgaagtgtttctcatgggaaatcactctacagggctgctcctgcctgttgtactacatcccaccacaggcagctgcgggaggacctctccatgccacagccacaccttagtctgagaaatggtagccccaaaccccaggaaaagcaatgtgactatctacgtgcttgctcgctccccacatgcctcccatggggctccaggatggcaccaccaacttatagcttcactaatggagtacacagtgctaagtgacattcttagaactccatcaagtgctaagagaaaggctgactgctaagagccagagcccagggagtacatggtctcctgggatatgtgacagtggtgaggggatcaggtaacacagcctaactggaaggagacaatttgggtcccataccaggccaaatccaggcctgtggggaccactatctggtcttgcattcctggccctaatcactcacagaagtcaggcatggtggtatatgtctgccattccaacacttgggaagtcaactggatgttgtcttatacctatagtgccagcactgagagaaggcttgatactgaaagatcaagagttcaaagccagcctgggctacataggcagatcctatcctcccaccccacccccaaccctagaaaaatataaaattaatactacattgacttcccaatggaggcctgagtgactccaacccctctatactgtagtgctccaggtcacctttgagagtttggacttggcatgagggtcagacctatttgttatctctaggctagcttgttcttccagccctcggcttacagctgtgtgaactaaggagctagagaactggccctccacgtgtttctgaggctgtctctttaaaacacaaaaagtcagatggaggctgtatgcacctttgatcccagcactcaggaggcagaggcaggcagatttgtgtaagtttgaggccagcttggtctacagggtgagttctagaacagccagggctacacagagaaaccctgtctccaaaacaaacaaataaacaaacaaacaaaaagaatgaccatttcccagcccaattacccctccaacagcacccaaaagtgttctcatcctaagaggtactgtactctgtggcctctacctgactttgatcaattccttgctctgcagtcccacctgaggggactttcattatttctttgtttctcaggcttcccgaggagcacctgaaagccatccaggattatctgagcactcagctcctcctggattccgactttttgaagacgggctccagcaacctccctcacctgaagaagctgatgtcactgctctgcaaggagctccatgggtaacggagagccctgagagaggggtgggggagcttcatgggtaatgggagcccctacccacccaggaggatggccacagcaagagaaagtgctcattagagtgaccctgggtctcctctctgtccagatgtctctgcagcactcacagtaattggcccaggtggagtctggaatgttccaggcttgttggaagctcttgctctcatagaatctgagctctaactgagctgggaaagttgatcatttgtttattccttttagggtattgggggggcacggatgtatgcatgctggtatgtatgcatgctggggatgtagggcctcatgtgtgctaaatacatgtgcctattctgtaatgttttcttgtttgtttttaactcaaattaattagaggcagtttctctgtttaaaaaaaaaaggtggaagacactgccatctcatttgtgttgggacttgagatatatatatatatatatatatatatatatatatatatataatttatttatttatttatttgtgtatgtgtgtataagaaagcgggggacactcatgtgccatggtgtgtatggggacacacgtgtcctgtgggggaacattgtgtcaggggaacaagcatgtgctgagggtggggggatatatatgtgccactgtggtgatttggttgggggagacatgtatgtgctatgatacctgtgtgtaggacagaggacagactcacacaggagccttcttacctgctgagctgtctcagcagtccaagccctccaggctgtagccactcttcctccttgctacagtcccacatccagccaacacataaaggctctggcaggaaataaattaaatttgctttgtgtgtgggtgctgacggctcaagtcttccggtgatggtaatgtttaaagcaagccaacatcagttctccagtgccgaagttattgaatgactgaccaatgggtaacacttaggatttttaaaaattattttgattgtacattttgaattacattaatcttaaaataaactacaaacataaacacactgtggttggagctgggcatagtggcacatgcctttaattccagaacttgggaggcagaggctggcatatctctatgactttgaggccagcctggtctatataatgagttccaggacagagagatcctgtctcacaaacagagaaaaccccataactatacttttattgtactggttggtctacagtgtaaagaattggcaaagaatatgaaagattacactgggaagaaactgaaagccatccagagtaatgaagcaaacccctcacaaatgtgggaaacatttcacatgggtgagggcttgcagccagcctgtgctaatttatgttttggtacgtgagcacttagagcagtttccagttctctggtgctctaccaatcttagcttggttaatattcagggatgagtcttccatcaccggtaatctaatttgccattctatttaaaggctcttaaaggcacaggcagtgcattgggtaaatgtggcaaataatttctttgacaaatcgaccaattgtcagattggcctgctagtcatttgtttcaatgagaactgttttttctcaaaggatgctcttgtacaccggctagaagcagggctgtcatttttataggtctctgtggtattttgttgttgttgttctgttgttgcaaatcatttatcactgagggaaaatacacacaaagggccctttctttaaaagtatacatgtatcattttgtgacagctcataagaagctgtttttttctgcctggacacaggtcctgacctgtgctgtgtccttgctaagctttgtcagacccttccacagctccccccaacaacgagttccccagtacctgcctcacctcatcactatggtgacagcagcctctgatgcgcctgactctctggcacattatggcagtgttaaaagcttccatctctctctttgctgaataatgaacctcaggttgttcaagagaccggaatgttcttcacctgcctgcacacatctcttcactttcttttatagatcaggtagggactgggcgtgtagatggaacaaactgttttccgttccccagccatctctgcaggtgcactccacataaatcaagtgttaaaagtgctttgattaaacaggacaggcgcgttcttgagttcatctgttcacatactgtctggcaagcgctgactgagggtctcctctgtaccctgttctgagaactaacaaaagacgaatcaacatacagaaaactgttatttagtgactgattaaactaacgaaggcatgggctggagaaataactcagcagttaagaacatttgctgatcttgcagaggacctgggttgggttcctagcacacacagggacagttccagtcccggtgtgtccttttctcacttctgtggacacaagttttacacatagtgcacacacatacactcacatatataaaacagaacatttaaaagtatgtttaaataacggaatcatttatataggttttcatttacataggtaaataggcaaaaatctgcattttattgtttctaagttttaatttatttttctctgtgtgtacatacgcatgcctccttatctgtatgtgcgtgcactgtgtgcatgcatgaacccacagagaccagaagagtaccacagattctctggagctggagtgattgataggctgttgggagccactccacatggggattcagagttgaacttcgttctctgcaagaacagccagctcttaactgatggcttttacctccagccaccttttcctcatttttaaaatttccttccttcctttttgagacagggtctcaatacttagctcatcccaacttgaccccactcttctcttgccttagtcaccacaatgtttagtttataagcatgcgtcactatgcccggctttaaataaactcacccataatcccagcactgaagtagacaaaagggaggatcgatggggctgactggccacaagccgtgcttcaagttcaatgaagaccctgtctcaagggaataaggcacagaggatagagccatacgcctgacctcctcctctggcctctacccaggcacatgtgcatacacacaccacacacacacacacacacacacacacacacacacacagagagagagagagagagagagagagagagagagagagagagagaaactttttcctctttttttttaaaaatattatttatttcatgtatatgagtacactgttgctgtcttcagacaccccaaaagagggcatcagatcccattacaaatggttgtgagccaccatgtggttgctgggaattgaactcaggacctctggaaaagcagtcagtgctcttaaccatctcttcagccccacaaagaaacttttaatgagcaaataattgcttccaagtaaatactactaatatatttctaaccatactatacaaggaattattaaagaacggataataggagaataaaaaattataagtcactttataatgctatctaatccatctagaacaaaaacactgtaataatgcaaaagagcgcagtgcctagattaaataaataaaatgcagaccaataagtaaactttatagcagcacatggaaatgacgaaattcctaacaaaaagctcaagatgggcagtttatttaaagtgaaatacaggagaaataaagcacagaaagatactcaaaggcatagaagttaacataggggggctggcgagatggctcaacaggtaagagcacccgactgctcttccaaaggtcctgagttcaaatcccagcaaccacatggtggctcacaaccatccgtaatgagatttgactccctcttctggtgtgtctgaagacagctacagtgtacttaacatataataaataaataaaactttaaaaaaaaaattaaagaagttaacatagaagcccactcaggaccccactcagtcctagagtatgacattattatggacattaaaaagagaaaattcagcagtagtgtgcatgcactgcatatatacacaaatccttgagtttcataccaaatgcctttagaccacttgtggctctgcaaacctgtaatcctagcacttgtgaaaaggtcagctcaggaactttgggaaggtcatgaaactcttgcccctccagaagggagaggctaattaacatttctcagaccacagggcgggaaccgacctgcgggtggggacagactgttgcccatttccagactagggaagtccttgtcacctcattccctaaagaccaatcaatttaaagggtgcactgttccgccaatcatattgtgcctagttgctgatgctctattctgcccttagaaaccgtataaaaactagcgaaggggtaccaggggtaaccccctctccttcaggtctgggacaatcccactacactggaacaataaattcctcttgctttttgcattgatcacagctccacttcgtggtaagctaagactccctggagtcttacattggcaaatgcaggcaaaagaatccgaaactcaaggtcatctaaaactacatagcaagcatgctgctagcctgggctccatgagaccctgggggaggggcagagggagaccgttcagaagacagtcaagatgttgcagcagcacaggcagcctggccaccagtgctgtcaccagacatgttaatgttggaataaagcctcaatcatgactctcccagttttataattggaaataagaaaggaaagactataggaacaactgtgttcagaacactatttataatagcaaagatctcagagtaacccaaacttctagacattgatttgggaagatctcttggcagcttattttgaaaactttacaatgttaaatatgtaaaaacaaggacagttttgttttttgttttgttttgttttgttttagggatatatattcatatatgtatatgaatgaaaacccaaacttaaaattccccactatgctttaaaggctttctgacaataacagaaagagaaatagagaatccataaaaactagttctgaaactatcaataggcttgacactctttagctgccaggagagctgaatctgaacacagggaaccccacccagcaccccaaatttggattattgttttattttatctttcccctacccccaagacagggtttctctgtgtggtcctggctgtcctggaactcggagatcctctgcctctctgcctctctgcctctctctctctctgcctctctctgcctctctctctctctctctctgcctctctctctctctctgcccctcgctgcctctctctgcctctctctgcccctctctgcccctctctgcccctctctcttcccctctctgcctctctctctgcccctctctgcctctctgcctctgcctcctgagtgctgggatttaaaggcatcagccatcacttccagcttcctttatcattttaaaaagaatttcctatgtgactactgtatttaaatcaccacacggccaatactcccccccaactcctcccaaatcccctctacccactcaaattcttatcttgtattctttatcattattatacatatgtgtatatatgtgtgtgtgtatatatatatatactatatactgctaatgagtaacatttagtgttattcattgttgcatgttttcaatgtgctttccaggaggctggggggatggctcagtgggcaaaattctagctgcacaagcctaaggaccagggttcagatccccaatataaaggctggctggacatggtggcttgcctatgatactagcatgcttgctggaagcaaagacagggaatccctggagacttagaatctcagaagtgatctgggctggacagactagctgaactggccagctctgggttcatcaagaaaccctacctccataacataaagtgtgatggagaaaggcacctaatgtcaacctcaaacccctacctgcatgtgcacacacatacatccacaccacacacacacacacacacacacacacacaccacacacacacacacacaaataaataagtaaataaataaaatatttagctctccagaccaaatcttggtgaaacccatgcatttgcatttgtgtgtgtcctacaaacactgaaggttaagaagcatgctccttagtaattttatagcagtttgcgtttccagattgaaaacagattctataggctacacagtgctaaatggattatgctcagatacagattgaaaaggatacagattgaaaagggtcggggtctgggccaggatgacgggccaactgatctttgccggggcttgtccttcagggaagggttacaggattcaccactggggtgtggcctatctgctgttaggacctgaattgcctggagtgtttctagttcccactagttgttgaactttaccttgaacctctgctcccagggaagtcatcaggactctgccatccctggagtctctgcagaggttgtttgaccaacagctctccccaggccttcgcccacgacctcaggtaaggggtttggatttggaaagatgcaattgctataggagggactctgaaggcagacagacgcaccgcctcctcacgttggctagtctaatataaacatcgcggtggatggtgaggatagactccatgcccttttgtgaaggcatttcctggcatcagctcctgacttcagacagtttcacccatcagacaaattgcctggtgttggaggaggaggtgagcagggccattcccatcatttctcctcagaaatggaaaggcaaggaaaacatgaggttcttcagacacttaatccctgggactgcaaaatggtggtgcccctcctccacagctgctcacggcggggcaggagatgagggccaaatgaagcatagatctagctattttttttttagtgccttcagtaaatttaaaatcaaataagggaagggacctagatctttatgttatggcattgttaaaagtgagaacttgtagccagggtgtggtggcgcacacctttaatcccagcacttgggaggcagaggcaggcggatttctgagtttgaggccagcctggtctacagagtgagttccaggacagccagggctacacaggtttctctatacagagaaacctgtctcgaaaaaccaagaaaaaaaagaaagaaaaagaaagaaagaaagaaagaaagaaagaaaggaaggaaggaaggaaggaaggaagaaagaggacaacatggtctaggggtcagagagcagaatctccaaaaacaccaacaatgcctgctgtaaatgtatgtcgttgatttggggatgttggcctccagctcaccatttcctgccttagcctccaaagtgctaggattataggcttgagccaacacatctggcttacgcctattgtgtgtggaaggggagtgctgagtgtgctcctgtgtttggtacttatatatgaatatatgtatatacgcatgtacgcatacttgcatgtgaaggccagaggccaatgtcagctgctctcatcttatcctttttattacattgtatttatttgtttgtttgtttgtttgtttcatcgtacgcatgcagccactcatgagcatgtaacagcacaggtatgaaggtagacttgcaggagtcagttctctccttctgtcacttgagttccaggaccacactccagcccccaggcctgggctgtaagagagccatcttactggtcctctactttgtcttctgagatagcatctagactcacggaacctggagctcatctagatttacattggctggccagctgatgcattttaaggtcaaatcttcattccatccctaccccacttccactcccagtgctggagttcgggacacctgccaccaagcccagtttttcctggatgcagaagctccaaactcaggttcccatgttcgcatggcaggcacattttcagttaagccttccccccagctcctttaccctggtctctgaatgggggggaggctataaatcaggctgctctcagacattaggtaggaaatagaccatatacatgaggaaagatattcacctgccccatgggtaccaggaagtgatgtccaactcctcttttgcttatcaggagaaatgctgactactacctctggtaattttgatgttgggaggaacagggacattcataggaccccattcctcgctggtgagagtggagacaggttttctgaagggcaggagatctgtgtagaaaagatggatgctgttttctgaagggaaatggaggtagagtcgacctgggagagaggggaggtgggggatggttgggaggaatgaaaggaagagagacggcagttgggatgtattgtataagagaagaatcagaaagaaaaaaagaaaagctacctgcacccttcaagtgttcctctgtgtgggaggctgtctcagggactacatgggcaccgagaggcatcagtgagggtaggtacttgatgttgtgtccctgaaaacaaggacaggaaatctgctgcatggcctaagatggcaaaatgtggcacaatcaagtaaggcccaggattctgtctgtggtgcagacctgctgtagaatgagctcccagcattcccacttgctgtgtggagacagcatgttgcagagccatgtgaggatgagggtccaggccaggaggatgtcaacccaccaccatgtagccagtgggctgggggagcttgggcccaccaaggagcttgagcagactgacagtgggttatgtacacaagtgggcgtgtcacacaaccgtgcaacacagagaaaatccctgtgatgacaacttctaaaccaccctgaggcaaaaggagtagacaggggattagagcctagcatattggagtcgagtggccatgcagctcttggaagcgtgaggaaggaaatttcctggaaggataggttgtcttcctagcagcctcgtcaatagatgtcaatgtatgaggtagtacctgctacaatcctgcttcttcagaagactgaggcagggggattacttgaacccagaagttctaggccagtatggacaacatagcaagagtctgatttaaaaaaaaaaaaaagtaaagagggaaaccaaataggtgacgtgccacactagtgtcttcctgctccaagggtcctgggcacatgagcttgcttagtgccagaaaagtcagaggaggagagggcagacagagaccctcgtctccacctcctttgactgactaatggggctggatataatctgttttacaaaaggacagcttttcagagctgtttctatctaaggttgctctgaatagccatctcgaaatatgccagagaagaatatttagaggcggcctattttggtctcccacaaagatttcacagggaaaatgtatttgtgttctatttatacaactaaaaatatgcatcagcccggggaaactggctctttgctgcctttgaagtgaaggatgggttaatttctaagaaagtaaaagcaaatgtagtgcaggcaccacggatgctgccagacaccagcgtttaagtggcttgaatggaagagcacaccccaagtatttgaagtaggtgaggagagagaccaggtctccagagttgggcctgcagtggccagggtaagtccaaggggcagtatgacgcagaacagagtggcaacctctaccagtagtagaattcaggtctcattcctaacctcccataaagcagagaatattgcactctctttctctctctctctctctctctctctctctctctctctctctctctctctctaactcacagagatcctcctgcctctgcttcctcattgctggaaataaaagcatgtgccaccacatgcagctctccttgtttggagagagaaaggcagacagacagacagacagacagacagacagagtataatgtgtgcagatgtccacagaactccgaagagggtgttggacccccagaaaccggagttctaggcagttgtaagctgtccagtgggtgttgcgaactgaactcaagtcctctggaaaactggaaagtactcttaaccactaagctatctgttagtcccccaagaatgtcttatcttgataggccttcagatctcacagtccagggggctgacctgctacaaggggtagaggaaagaaaactggctccaggcctgagttcagagtgacatctccagagttcttccttcccttgccgtgtagtaatttcctaccctacacgaggagaaaaggaacagatatgtccagtatgcctggcatcttgaaagggcactgactcttgccgctgtagagcccctttccttggggagtgcagagaagtgctgctagagaggttcaaaagaaacacaacagtcaaaatagttgctggccaagggagggcatgtgccctgtatcgggcctgcaaagccctttcaacagtgtagccaaggccatgtttgacagtacaagcctgtaagcccatgcataagcaagggctgggataaagggctactgttcaagttagttatatacacatcaagtttgttcatcttacatccttaggtaagggtgggtagcttgttagctccacctctccagcaggaaagcatgtccacggtaaggtagatactgtagagtttagctttgttttgacctttctttcttcgttgcttgggattgtacttgggaccttcactcatgcagcccatgtttcagccttgcataaatctataaatatatggttggagtcaggtccagcatatgtgggtaccagtcccagtctggtatagcaatttattagctatttgtctttagacaagtaactatagggttctgagctgtaatggccaccaaacaggtcctttaagtatctttattggccaaacaccagggttaaaacactgaacacagtatggtggtttcaactgtcatggagtccacattctagcaggggaattttacacacaactcacaaatgatacagaccacagcagggtgatttgacagagacaacgaagggcttgcctgagctgcagtagtccgggagcagctcttgatgggatctgagatctagatggcagagcgcccttcctagatgaaatccaagcctctggttactcctcagcaagagtagctggaggaagaccagaagtgaggaggaaaaggctcagagtggagcatctaagcctgcagcacctccagagacaggctttggagcttgcctgtggttcagcctactgtgggaagagaccagtgagggttttaagctgtggggatgggtatattttggaaggcgaacaagaacaccagcagctcaggcaatgtaggagggcgggaaggtgatggatgatcgaggacggccccagagattttaaggactgtgtaggtggggtgagaagcactgtgggtgaggtggagttagagggaagaagtgaagatctatttttttggccatgaggaatttggggtgcacacacacacatacatacacaccccatcatgtccttgtctacagacggggtgataatggtactggaaggtagaaggtggaaagaggaccagcaaggaaggtatccagtgcccaccccacagctcaccctcagacagaccttgttctcattttcattacccaggtgcccggagaggccagtcccatcaccatggttgccaaactcagccaattgacaagtctgctgtcttccattgaagataaggtactcacaggtgtcctgtagcacctcctgatttctctgcagtcccctatttgcactgagtgtccaagtcccaggttctgcattcctatgactcgctgacattcctgctgtccacaggtcaagtccttgctgcacgagggctcagaatctaccaacaggcgttcccttatccctccggtcacctttgaggtgagtgattctggagcagtatttggccgtcggttcctgtgaagagagtgcttttgatttaggctgtgaggagcgaggaactcaatggcagagaatagtagtcaggccagccacctcgctggggcagtgggtaaacactaaaatcgctgcctaaatgcaccagtgaagtacccctcatgggaggctgcaacgaaagagctccccattggtccaagatggtatgaacctccttaaaataggaaggagaagtgacatcaaactgagggccacacgttgcctataaaatggtgtctggtacaggaacagtgggagaaaacaattatttttgttttgggggtttggtttttttgttttgttttgttttgtttttttgttttttttttttgagacagggtttctctgtgtagccctggctgtcctggaactcactttgtagaccaggctggcctcagactcagaaatccacctgtctctgtctcccaagtgctgggattaaaggcgtgcgccaccaccacccagctttttagttttttgtttcttgttttttgggggagaaaacaattcttatgatttccttaaaaatgactggcttaagtggagacctagaaaggtggttctttctctttctttttctttctttcttcctttctttctttcttccttccttccttcctttttttttttttttttttgtttttcgagacagggtttctctgtatagctctggctgtcctggaactcactttgtagaccaggctggcctcgaactcagaaatccgcctgcctctgcctcccaagtgctgggattaaaggcgtgcgccaccacgcccagctccttctttctttttttaaaggtttatttattattatacataagtacactgtagttgacttcagatgaaccagaagagggcgccagatctcattacagatggttgtgagccaccatgtggttgctgggatttgaactcaggacctctggaagagcagtcagtggctcttacccactgagccatctcgccagcccaggtgtatttttcttaatggacccccatgattcttgtacttccagctcaacttggtcctgcctttatgagacaccccactgcccaccccaaagccctcttcatccctgagaggagtctcacaaaaaacagatttccatttcttcttaccaccttggttttttaaatttcacatttatctatcatctgtctgtctgtctgtctgtctgtctgtgtgtctgtctgtctatcgtgtgtgtgctgagtgagtgagtgagtgagagagagagagagaatatgaaggaaaaggacagcttgcaagagtggatctctcctaccgtgtgggtcccaagagattgactccagattgccatgcttggtggcagattccttttcccacaaaaccatcttgcctgacttctctactgctcctggctttaggggggcttttttgtttgtttttatttttgttttcatgagataggacccccaaatagcctccttgagtctacctaaaccttccaccacacccagcttagcttttcattttctaaacatgtctgcgtaggaatcgtcttcctaattattaccagtcgatgttcaattagcggttgtaacaaggccccagagtgtgaatctattgctaagattctttccggaagtagatgacacatcagattaataagtgttgtcaaactcatcagtgaatgaaccatttcccaacagttcatctgtttcctccaccaaatctgttggtggagcagctgggttacacatgggcgagctgggctcagccgcggtgccagccatttttccagcatgctatggaatataggggtgttatcaggccagggtgggaaaagcctgggaatagcgagccagatagcctcacccgggacattgaccatcccagccatgtagataaatcagagttgacatcaccaaagccagccactgactgcatgtcttatgctctgaaggtgaagtcagagtccctgggcattcctcagaaaatgcatctcaaagtggacgttgagtctgggaaactgatcgttaagaagtccaaggatggttctgaggacaagttctacagccacaaaaaaagtaagccacccaccccaaccctgcaacacacaccccatgttctggttcctgtgtgagagctcttatgaatgaccagtgactagatgtcaacagcgactgtcttagttacttttctattgctatgacaaaacaccatgaccaagaccacttacaaaataaagtgtttgatgtgggactcacagctccagggggttagagcccatgaccatcacaatggggagcatggtaccaggcaggggagtgtggcgttggatcagtagctgagaaactacgtcttggttcacaagcataaagcagagagggcaaactagaaatgccgtcagcttctgaaatcacaaagccccgcccccagtgccacatctcccccaacaaggccacacctcccaatccttcctaaacagttccagcagttggaaaccaaacattcaaatatatgtgtctatgggagccattctcattcaaaccacctcagtgattgtcagcaaaagccagtcccaccccctttgcagggtagtccctgtccatgggtgctccccaagccgctccctgcctatggaagtcttttcctgatacaagggcactccttaggggcctcccctgcatacttagccgccagccctgaaaatgagattataaatttgtcacaactgccccatagcagtcctgtgtacacacggtgatttcatgtgtctgtgtgactgtcctcttccctgcaagcatgcagttacctcactgtgccgggacaggccctgtactcacggtattgttggcagagctgctgggttaccacccatagcaaatcactaaccctgtggagctgaagtatatgaagcctgcgtctaaatggtccaaactctcactgccgctctgtcacttggcagctgggggaccttgacccgtctctctgaacctcagtttccagttctcagggatgagaaagattgagtgaacaacatgagggacagcccaacacccagcttcctttccaccagctctcctcatggacaccgttgagccactgcagctcttctgtctgttgtactgtccagtatttgtacacacagcaggaaagttccccatgacagctggcggagaagctgctgtgggcagagcagcatccagagaagcaagccacccatatggtccctgcccacatgcaatttgtattctggtgcattgagggtgaataattctcagctaaatctaccaatacagtggcttcagctactcggaagctttgaagaaatcaggacagcatgataggtaaagatcctttgggaagagagaatggacagaggaggctagcccttttaagcaatgagcttgtgagctgagatctcaaaaaagctactggttggacaggtgcctgagggaagagtctcccagcgagtgcataaggtggagtgacaggatcttaaagaacagaaatagatgtggggacagtgtagaatggaataggagggcaatgggaatgttgaatgacctaacctcctgaagaggactttggaaaaactcccaagcaagaagttttaggctagaccttggcgctgttgaattatggatgcaggtggttctgagggtagccctgggtgaatgctcatctccatatggggttggacaagcccaccttcaaattcaggttctacctcctgccagctgtgtaacctagcctctctgcgcctccagtctccatctgtaaaatggggatgaagacaacagatgtcaggtggagattgcatgcttatgagagcaacttgcacatctcctcactcctctaagttgagtgagagaatgcccaggcaggggctagaaaggggacctgttgatcctacacctcagcactggggaagaaaagggctctttttttccctcttgcattttgagttcgtggcagaagccaggacagattaccaaaagaaaggagaatggattgatcacatgtaagtctcgtgacgtggaagcccttacagggaaagcaggaccaggaggatggctaagcagagtgctgtagccagatttccaaaaggtgggaagctagaaagtgatagcagagaggggctgggggctaagtttagggcaatgggaagagcttggccaggcccgtaagctcagactcctctcagtcttgtttgtccttggcaataaatgccttcccttcgggtacagagagcatacttgtcacatgagggttttatctccagcttagaggaagaccagaaaatcctttctccctcctcatccttcttgtcagggtgctatattttggggtactgggtcctcagctccatcagtaaatagcacaggaggtattagaagtcaagaactgagtttcccagctcagtcactgtgacttggtttggtgcccattctggtccttggttgcatgttgctacaaggcttaaaagaatgaatatcaaaatgtgtgatgcctggcgtggagagagtgtttggtgcaattggccactggcggaggagtgtcattggtacaattatccttccatgtttctgtgaggacagaatggaatgggggtggaaggtaaagagaggaagtaggaaagacagcctctggactctggagtagcaagctgtagggaaattacagagctgagcttacatcatcaggacagggctctctctgccttactgtgtctagttggggagtgattgatggtttgagcgggaaggctatgccagaagccgttggcttagaatagtgcatactgcttgtacagggcaaataagtccagagattcattgcagcagggccactggtgacagctacttagagcaaactgaactcccgagaagctctgagagctagggtttggtgctagctgggtgctctggctactgggatggctgtgtgatgcactgcccgtgctacatagatttacccattccactgcgtgcccatgcttcagagcctcctgtgtgccatggtgtttatgtacagctttctctgtcaaattaaaataaattaataataaaggaacaatgttgagaggggagagagagagagagaggatgtggatagatattttggggttttttttctgttggttgttttgtttcaagacagaggctcatgtttcctatgctagggttgaattcgctctgcagctaaggtgagtcttgacgcccattgatcctgggtaactgcattccttacataatctagatctcattggttttaggaaagcaggaaatacaaatgagcacatgttaaataaaatcatatgttgtactgtactctcccagacagccttgtactgtactctcccagatgcttttaaaagtaacaatgctaggccacatgtggtggtacagccttttaatcctagcacacagaaggcagaacaggctggtctctgtgagttcaaggccagtgtgatctacatagtgagccccaggccagccagtgctgcataataaagaccctgtcttaaagaaacggggaggaaggggaggtgggagaggagggagggagggaagaaggaaaaagaaaaatagtacaaactgagtgttatggtacatggctggaatcataacaccaaggaattttgcacttttctttttataaatttacattgtgtgtgtgtgtgtgtatgtgtgcacatgtgtgtatgcacttgcctgtgtgtgctcacccatgtaaacatacatgcaaatgaccagaagaggctgtggatcccctagagctggaattacagttgtaagctgcttgacatgggagctggcaaccaaactggtgtcctctacaagagatacatactctcctaaccactgagcctcccagcccacactccaagtgagggtacacagtgtttcttcttgtgtcacaggaaaggaccaatgctgagtgtttcagacccacaaaagccagaaatgtcatgattcagcctcatagttgcaaatctaagctgtcctctcttcagctatgtatgtgtgtgtgtatgtatgtatgtatatgtggcttttttatgaaacctatctttaattggaaaagtctcatctttgtgtctttattcttatgagcttgcacccatgaaggaagagtgggaccaggcaaaaatgagtgagaagacaaaacacacgatgggatgagttacacagataaagtgaaataaggacatgagcctatgaaataagctggagagacggcccggcagctaagggcactgggtgttctttcagaagacccaggtttggttccccccctcaagacatgacagcttccagccatctgtaaccctagttctattggatcacatgccctcctctgacctccatgggctctgcgtgcattcagtgcacagatatgtgcaggcaaacagctgtacacataaaataaagatttgaatcaaagccagaaatccctttaatcccagctcttgggagacagaggcagggggatctctgtgagtttgaggacaggctagtctatatagagaattcaaagacaggaatatgtagagagatgtatgtatgtagtctcaaacaaaacaaaatagaatttatttttaagatttattatatgtaagtacactgtaactgtcttcagacacaacagaagagggcatcagatctcattacagatggttgtgagccaccatgtggttgctgggatttgaactcaggacctcgggtaagagcagtcagtgctcttaaccactgagccacctctccagccccaaaatagagtttttaatttaaaatatttttgaattaataggaagagggaagaaaaaaaaaaaagagttctgtacaagggttaaaggcagggcatcaggaattcaagccagccgtggctacatacaaccatgtttcaaaaatccaaaaagaaataaacagattttcaaagtaaaaattttaaaggactggagatacatctctgttgtccagtgcttatcaatcatatgcgggccccttgtactgccgaaataaaataaaaacaaccagaaattaaaaacttgagggaaggaagctcttagaaggtgggtttggtttgggtttttgagccagagtctaatgctacctcggttgcctggcagctttcccacaagagtcatatggaggtactcaatgcccccacctctgtcagtcatgacaagtctaccagggactttgaggagggtgctcaaagtggttatttgtttgtttagctaattagttttgtaatcacagtaatcaaacccaaggacctatgcatgtcaggcaagtggtagccaaggaactcactcctcatcctcctgcctctacctcccaagtgctggttatggcgtctgccagtaggcctagctccagggaagggcttctcaaaacaaaacaaaaacactccaatagcaaaaaatgacaaatgggattttataaaattaaaaaactctgaccagcaagggaaacagttagcagagtgaagagacagaatgggagaaagtctttgccagtcacacactgacaggggatcaatgccagtgatggcggcacacccgcgtttatcagagcgacatttgttatagccaagttatggaactgaccttggtgcccaacattgaaggaagaaaatgttcctgaacatcatgggaggagtattactcagccataaagaacaacacgatactatttaccaaaaatggatgggactggggggtgagggggtacaattagggaagaaagggaccaggacagaaacaaagagtaactgtggaatgatcaaagagtgaatgaagaggttacaatgaatagatatgttaatatatgctcatttaaaagtcaaacatgttttagaaaataattcttggaaagctactagagatgttaccagaatgaaggtgtaaacaaagagagagggagacagaatgagagagagaagggtccaaggagagggacaaaggagtagatgccagggtgctgaaggaggaaggtttccaacaggcctcaaagtcagcagacctagaggctgtgggaaagcacttactcataggacagacagcaaagtgcttagtcatgtggggaatgagaggggagtctgaatggaggaaagcacatagcagggggttaggagggtagcttagcatgcagtgtgaccctccgtaccaccaaaaaagaaaagaacatggtcattgtacagttataaacccaggaaaggccaatataatatgccttttctcatactgcccatactcaccccatactcctccctctaactcctctcagacccaccctcaccccctccctcctcccaacttcctgtcatctcactgagtccctgtgatggtttgtatatgcttgggccagggagtggcactattaggaggtgtggctttgttggagtctgtaccctgtaccatcacagtgggtgtgggctttaataccctactcttagctgcctggaagtgagtattctgctagcagccttcagatgaagatgtaaaactcactccttctgcaccatgcctgcctggctgctgccatgttcctgccttgggtgataatagacgtaacctctgaacctgaaagctagccccagttaaatgttgtccttataagagttgccttggtcatggtgtctgctcatagcagtaaaaccctaactaagacagaaattggtatgtgctcctgggtatgagaccatcccctgggacctggcactacctggagctacatccttaagaaacttgactttctctccccagcagtcatctgccaatagttcctcagctaggggaggggctcatgggcccaactcagggaaacgtttttaagttacataggaaaaagaacaaccaggactggtctgtcaggagccttgtcgtggtcacagtgacataagcattgagtgtttatgtagctaaaatacaacaataaaccaggtggtcatgcatgccttttatttatgtttctattttttaagaaacatgagataaagttaaaagagttcattgaaaaacttgaaattagggctggagagatggcacaacagttaagagcactgactgctcttccagaggtcctgagttcaattcccaacaaccacatggtggctcacaaccatccataatgaggtctgacaccctcttctggtgtgtctgacagctacagtgtattcatatacataaaataaatatatcttttaaaaaaaaaaccttgaaattagttgccttcagggggcaggacattggagggagcagagtggaaagatactattgccaacaacaacaaatcttaaagaataatttgattcatttttaattttatccttattatttttaattatgtatgagtgtatgcctgggtatgtgaggtgctccaagagaccaaaggagtcgcctggagctggagttccaggtggttgtgagctgcctgatgtaggtgctaggaattgaacttggatcctctgaaagagcagtacatgttcctagctgctcagccgcctctccagcccctcaatttttgatctaaaaaaaaagttgaaagctacaaattttctgtgaatttttcacaaattaagcaagacagagaaaaggtagggaggtgaagtggcatggaaggagaaggagaaaaggatggacagtctcatggtgtgtggagcaaagctaactctagctggttctgggtaccacactttaaaatgagcccccagggcttagcccaggtatgtgggggccagattggggcttgggtgcctcgggagcaatggacagtggacatagcggataaagaaacaaatgcatgaatccataaacttctgtttttctgaagtcctgcagctcattaagtcccagaagtttctaaacaagttggtgattttggtggagacggagaaggagaaaatcctgaggaaggaatatgtttttgctgactctaaggtaagtgacaatagacatctaatgtgaggtgaaaaggatcatgggccttgaggaccgtgctgtgctaccagggctggcgatcattccctccctgggttctacagcaaccttttcattcctgggaagctgaacatagctaggtaagaaccaggctctgggccagctgggacttgcagagttttagttccagcgtcccaggatcacaagtactatcttccaggaacaaggagacactccttgtctcaggcccagtgctgagctggccaggatcctagcctttattaacagtgtctcttggcgagatttaggagccctttgggtaagctctggatttaagacctgaaccagtgctcccctgtcacttctgtgtgacttctgtttttaaactctgccctgctctgctaaagtaacccctggatgtgcttcatccaatccagatgtgtggtctctgcatcttgattgctcacctcagaagtcccctcaaatgggctgattaccacacagaggccttgctgttcacgctcagtgaggcgctaaaagtggatagaagaacagatagtgtgggtccacatgctgccccacctctggggtgtgggacatgccacttaggaaggtgaaacacaggagtttgcctgtgcttatcccatcctactgccatcaggtgctgggcttcagggaagaaccaagacaccattctgggggtaggaagatgcaatctaagataggggagattgaagttggggtttcctgacttccgggcctccagttgcttctggagaactgcacagagaagtcggtaatgaagtgtcagggattcatgggcgacacagcgtgataagggggagggggggctgtgtgtgtgtggctgagttgcaaggaattgagagcagagttcagggagggacccacaggctgatgactgggactagcccagggggccaggggaatgggaactctgggttagcttatcaacaaaggggtgcagatggactttctatgggagacttaagcttcggctctatagatgaaggccttccccatgctccccatggcagttctcaatgaaagagacagtccttgcttccaaagtgttattggttgttcatcatggaaaatggctgcttaccaacttctctgggtgggccagagattaaataccttcttcaagaaggaatgtcggttcaagaagggacactttgtggctaaactctcagggcctctaggtacctacctcattagtatagagaattctgttttgggctatgtgaacaacatggggaatatggtatgtgttccaggccaggaatctggcagggagcagggaggcacctaccatcttaagtgtgagtttctggagtcttctgttctccattaccaagtttcctggcatggtgaactgtcccacaagatagggcacatagcttagcatgtgtggctgactcacgggtcagacattggagacaagctaagggaaggctcccagtacagtctcatagaggtaacccccaccaggttcccaaggcatctactttcactcatcctgggagggaatatgaaggtacagtggcaggcatgagccgacgggaacagtagcaagagagtacagaaagtagagaatgttcacatggttaggcagcattctaggcttcaggagcataggtcccaaactggctatttctgtcccattgctgcaagctcagtggggttgagacactgacccttgagccatcgctggttaaaggcaggtttcaataaagaagctcttgttatgtcatgatgagctggtatctttagagtgggccactgtgatatttgcaatgatgaggttgaatcatatgaaattgctggctttgaaaccaggtgagggacaccaggcatccggcctttcatattggcacttgtcatagttagggttttacttctgtgaacagacacaatgaccaaggcaactcttgtaaggacaacatttaattggggctggcttacaggttcagaggttcagtccattatcatcaaggcaggaacatggtagtgtctaagcaggcagacatggtgcaggaggagctgagagttctacaccttcatctgaaggccactaggagaagactggttttcaggctgctagaatgaaggtcttaaggctcacactcacagtgacacacttcctccaataaggccacacctatcccaacaaagccacacctccaaatagtgctattccctgagtcaagcattcataggactgcttgaaattttgtgtgttctcatttaaagtcagccaccctattgacgacagccctctagactaagagagagacgagtctcatgctccgctgtgagcataggagaaatacatgtaagagatcagggaggcccttttcctaacatgccctttggcctttgcttagttttatttaaaaaaattattttatttggaggtggatgcatgtgcctcagtgcatgtgtggaggtcagaggacaacctgcaggagttacttccctctctaccatatgagtcccaaggattgaactccattgcatagtgtatgtcttcactgcggagccattgtgctggccccgtttctctacattttctctcaccaaataagtaattcagtcttacagaaataaaataaaaacgttgcaaccaaaatagttaaaagtttaaaagaaaagcagcacggccttcactgctaaccggttctggacttccccttccggtcggcttaagggattattgtcgtcatcgtcgtcatcgtcgttgtttgtttttatggtttgccttggtttgggctttcgtgagacaggatttttgcaatattgttctgtctagcctggtgctcactatttaccacagattggccttgaactcacggcagtcttcttacctcagccttccaagtgctggaattacatggccatgccccacactttttttttttaaagatttatttattaattatatgtaagtacattgtagctgtcttcagacactccagaagagggagtcagatctcgttacggatggttgtgagccaccatgtggttgctgggatttgaactccggacctttggaagagcagtcgggtgctcttacccactgagccatctcaccagcccttttttttttttttattgcaatggtgatacacattccattttgccaagtggcattttataaaaattatgtattgtggccacctttctgtggataaagtaattctacaccataatgttaaaactctgtgtgtgtgtgtatatatatatacatacatatatttgtgagtcccttacctgtgaacatgctccagtgctctaatgtatcttggtgcagttcctcattttttgcttcaaatcataaccttgcagtggaacagctgggtccaagaagccatatactcatagctttgaatgcatctctccgggtgggtgcgtctctcatttatacacacacacacacacacacacacacacacacacacacacacacacacacacacaaagacctttcagtatttttttaagctgaccacatctttgtagaggccagtgtgcagggaggtggaggttctgctaagtgtgtctgtccttcctccctcagaaaagagaaggcttctgtcaactcctgcagcagatgaagaacaagcattcggagcagccagagcctgacatgatcaccatcttcattggcacttggaacatgggtgggtccctgtgccccctccatcctaccagctctacttgggtccaccttcctgcctcagcttctacaagtggcacaagggggcacctctatctctcagccatagtcctggtctcaatcccatctatacaacctcttttctagagaattctacctttgtcagaggatgatgaacaagtaaaataggctttaggattagtgcaccccaaaagtagccaaagtagcttactctgcagtttaccaaggggccccaggcctggaataaagaagtctggtccttgcttatctagggaggaagtgaggggagggatagagacatcagtggatgagtagatggatggatgggtaagtggattgatggatagatcctagtggccctaagtttaagtgccatataaataatatataaggttttttttaaagatttatttattatatgtaagtacactgtagctgtcttcatacacaccagaagagggaggcagatcttgttatggatggttgtgagccaccatgtggttgctgggatttgaactctggaccttcggaagagcagtcgggtgctcttaccaatgagccatctcgccagcccaatatataagtttttagcatatatgtgtagttaaaagtaaatgaacaaaatatgtatgctaaatataatggtagcataatcatatggtaaatatgtaagtgaaatgatcatttggtataagtactcaacaataaaagctaaggtacaaaatatatattggattaaatatatatcatatataacaattaccacatattattatactaaatatatataatagtacattatactaattgtcaattatactaaatgttttgggatatatattgagctagaattaaatttttaaaatgtccagttaaatctgaatctcagacaaccattgtatggggcatacttactctaaaaatcacccagccaccagaaattcaaatttaatggatatcttagtctatcattaactcctcaggcactcaccctcaggtgttcaggcatgtggcacaaggacctttctagttctcttgtccgtctgtgaagagttcttttggttgaacatggcgggaggtcatcccattgctgcccttccagcagcaaacacatgggggcgcccttccaggagaagtcacatggaggcactaggcaggagccctgctgcgcatggcaggtctccttatgagtttcacttagagccccccggggggggggggggagtttggcagagatcattatgtggattattggcttcttgtctctctgttggctgagggacatagatagatctcctcagggtgtggggagaatatctacccatacttagggtagctccagaagtagattacccatcattccttgagggagctgcttccactcatttttcaaaacggaagctgagacaagttgcagccccctcctgtttctctgttgagcacctcttcatctcagttcttagctgacttataactttcctccaggaagcctttggtgtttcctggctgcacggtagaagcctccatcccggacatgtcactcacccctgaaaatgtaaactcctgagtgacaaacggggccagatttcatgtctcactgccaccctagagcctaggacatgtctggcaacctgttcagttagatcagggctccagagaggatctcccaggctttccatcctttgaaatgtaatgttatctttacaacaagatctcactgagctctgcccatagctaacatgtaaggtggtatcagcactgagtcctccatctagggcatttgcccccatgatttttgtaactatgtgattctcctcttcccctgttttgaatccccctaatgcccttgacctgacctgtctttgactcattcggtgttgggttttgctgttgaaggtaatgcaccccctcccaagaagatcacgtcctggtttctctccaaggggcagggaaagacacgggacgactctgctgactacatcccccatgacatctatgtgattggcacccaggaggatccccttggagagaaggagtggctggagctactcaggcactccctgcaagaagtcaccagcatgacatttaaaacagtgagcagctggccaggcctggggtgggaagacagcagactctttcaagcattccagaagtcagacaggatacttccaaagatgtataggattgctcaggggtaccccactttcagagccacagatgtgcattgaggtggcacccttacaagttgatagggtcctgagtccgccatcttccctactcctgcttaaaagaataatatcgccgggcgtggtggtgcacgcctttaatcccagcactcgggaggcagaggcaggcggatttctgagtttgaggccagcctggtctacaaagtgaattccagggcagccagggctatacagagaaaaaaccaaaaagaaaagaaaaagaataatatttacttcctagatgcattttcagtcccagttctcatctctgaggtgctttgtctcatttctaggcattgttggaagttccccctgaaagctaggaaatacagacagggtgtcttactcccagggtggaaccggggtgcatgttcagggttccaaaggtgtacctcagtcctgtgttgcaacaccatctcccaccaccaccaccaggttgccatccacaccctctggaacattcgcatagtggtgcttgccaagccagagcatgagaatcggatcagccatatctgcactgacaacgtgaagacaggcatcgccaacaccctgggtgagcatagagggaaagccattcctgtgcatgctcctcctcctcctccttttggcaatgtcccaggataaactgtgagagtcctgtcctgggatcccttcgtctctagcaaatccagaaggttttccttgcaaaaactcatccagggtctataccagctctggcaatctggctaaaatgtgggttctgtctcagtaagtctcagatggtttccattctgacaagctttcaggtgacatagtgccagccagtccagggatcccacttggagaagtgtgtgtacttgtgtgtgtccgtattttggggtgtgtatatgtgggtgtttatgtgcgtctgtgtctgtgtatgtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgtgtgtgtttatgtgcgtttgtgtctgtgtatgtctctctgtgtgtgtctatgtgggtgtacatgtacatgtgtgtatctggggtgaacatttttgatcccacaggagtggatcctgagtagatagtcattcactcagttgggggaggaggcacctcccaaatcccaaggctccaggctgctccacccctccttgtccctctctgctcagtatcatggcccccaacatccccatagccagaaacagatgatagcattctccttcctttctcctatgtagaaaatcacaaggctgctaccacctgccagtcactggcatgtcccacccccgctgacctatccactctcctctcttcccttggtctgcttctctttctccttgctaacagcattagtaccctttacctcacctcagcctctgacccctcggcaagcctgcctgcctgcctgacattctgcttcttctctttcttcctccctcaggaaacaagggagcagtgggagtgtccttcatgttcaatggaacctccttggggttcgtcaacagccacttgacttctggaagtgaaaaaaagctcaggtaatgggagccattccctccatgcacccagacagccttagcctcgcacaccctgatgcttgcccagcctcaggaccttctggacatctgccgtctgagcaaagacaactgtagtacagtagagacctcaggtatatgtgactcttgtctggaggacagaaaacatgttgatgtcattatcaacctagtgaacccctttgagcctggttcatttctaaagggaaataccactcaggagcatcctatagctcaaagctcactttgtctgcatgtgcctctgtcatgaccataaccagctatttcaattgctgatctgtttcctaatgggtagattcctgcatggaatctagactctgtgttcccagttgggtttcccagacacactcaggtgaccactgggacagagcttgagtctaagtctctgtttcccacctgttgcctctgacttcctactgtcagatcaaggacagtacccatgctagatgtgctagatggcccttgttttcttgaattaggagaaatcaaaactatatgaacatcctgcggttcctggccctgggagacaagaagctaagcccatttaacatcacccaccgcttcacccacctcttctggcttggggatctcaactaccgcgtggagctgcccacttgggtaaggagactccaccttgggtcgagccataggggacagaggctctcaggagtcggctgtggacagaaaatcaaagagacatgtgaacactcagaaccaggctaaggtagcagctcttcgcaggatctcaatacagctcctgtcatggatcctctgcctttcttcatagttcccttctgcttgtgcctcctttgccccaggacttctatggctgccccttgtcactgtcactgtctttccagtcagccagccagccagcagcagctgggcttggtcaggaagcacagacatcttagggggccagctcacttatctcaacaaaccatggggtcactggtggatccaaggcattgcttgcctgcctacaggaaagggaccaacagatgtgaccacagggatgagtctagaacccacacagttgttttgcatgctcataaaagttgaagcaggagacagcgtggggtggaacagtgagtgcccttctgtctagaaatcagagttagcagcctgcttccctcccacgcagggactttaatgtattcccacactcgagtcctgggagggacaaaggaagcaaagcaggagctgaccctgttttatagtcaaacacacagagaggctagggaagcggctgagaggagattgcacatcattcatagctgaggttggacctcagttgtcccttatcccaagcatctctccatatgacccatgtcccttggtctcctttgggcttccagccaccatcacagctctgcagccagctcaccatgccttcctcactagggacccccaggggacatctttcctccctgggaatgaacaggcaggtaggagcatgagaagagtttccatcctaagcctcctgaggaaggcctgccacagctccacgctcaccagggcagaagcccagagcctggctcaaagccacagagaagtagacagaagagagaactgctggagcccaaggccagtgaattccccagttctgcactgtggcaccaattcttgtaattaggctgagtggttacaaagagggtgatgctcagaagcctcagagccagggattccccaccaccactagtggacttaacactagatgcctttgttcccacagaaagagccagagaaagtaatggagaaatactacctgtctcatgaagtattgctaacaatggtgtttaaaagctgatgtataaaaacaacagatctacatgcaaatttgggctagtctgtagctaattatggtgatggtgcagcccttctaggctttccttatccttataaagtgattgcgagtcaggaatcagcatcccagacggatgtggtcattgggaagtgtggtgaagaacacgatctaacctggcatgtccccgttccaggaggcagaggccatcatccagaagatcaagcaacagcagtattcagaccttctggcccacgaccaactgctcctggagaggaaggaccagaaggtcttcctgcactttggtgagggcacactgtctctcctttgcatcttttccttccatcttctctctacctggacatcctgaccaaggacagggctgtgtctgcagaggagggaagctggaggtgctagggaagccaaactgatggccatgcgtctgtttcagaggaggaagagatcaccttcgcccccacctatcgatttgaaagactgacccgggacaagtatgcatacacgaagcagaaagcaacaggggtgagtcctcccagaagccactctcctgccctgtcccacctcctttacccaactcactattccatggtggttcctagaaagtgggaagtatcctacctcaccagacaacagcaaaaacaaaagtcagaatgcacatacaggggccagggatggaactcagtcatagagtgtttgcttagtgtgcacaaagccctgggttccatctcagcatcaagtccagcatggcaccatctatctttgatcccaatactcaggaactagaagtaggaagcaagaagatcaggggttcaaggtcatccttgactacaatgaatttgaggccagtctgagctgcacgagactttgtcctcccaccccccaaaagaaaactttcacatgctgacttatatcttggtacaaaactgccaccaacttgtacattaaaaaataatccaaaaagctgaattaacaatgaccctattctaagatgctgagagaattttaaagcattgtttcttttgttttgttttgttttgttttgttttgttttgttttgttttgttttgtttgaaacagggtctcactatgaaaatcctggctgtcctggaacttactatgtagaccaggctggcctcaaactcacagagatccatttgcctctgccttctgagtgctggcattaaaggcatgcactactatgcctggttactagtaaggataattttatgtgtattagtggttttgcctacatgagcatctggtgcccacagaagctgaaagggggtgtcagatccccagaaacagaatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcaagacaaccagagatttaagtactgagccctctctaaaacccttttaaaagacaaaaacaaagatacacacacacacacacacacacacacacacacacacacacacacacaccccacacacacagttctgttagctgaaactaaacaggttctcatgcatctgagtgtggtggtgcatacttgtaatcccaatacctgggaggcttaagcaggaagatcataggttcaaggccagcctggactacatggctagactccaaaacaccaccagcaaaactgatttctttgcttcaggacttagaatttcaaaacattgtagttctccattgataaagccacaggccaaagtaaaccggtcacagagtgactgttaatatgttaataaggccactcatgttgaaaggaataatgtttggggaaagatgcaataaaatactcttgcagaggacctgggttcagttcccagcaccaacatggctgctcacaaccaactgtaaccccaactccaggggtctaacactctcttctgacctttgtgggcactgcacacatgtgttgcacatgcatatatgcaaacaaagcactcacacacataaaataaataaatatattttaaatagtttagcactatctgagggtaaattccctaaagaaccaatgacagctgaacttgatgatggccgatgtcctcattatagggtggttttgtttttaataataacagagaaagcagcacatgtgctataaaactgtcatgttacattgcagatgaagtacaacttgccgtcctggtgcgaccgagtcctctggaagtcttacccgctggtgcatgtggtctgtcagtcctatggtgagtggaacacggtggggtgcaggctaggttttgggttctgaggacagtagcaagccaaggggcttcagtctgcttctccataagatgagcgagtgtcctgaaagagtttgtgcatctctatcccccttgggctgcagtgtaataaatcccgctcagagagagacactttaactagaaaggactttttgttgttttctttttctttttcaagatttatttattttatatatatatatatatatatatatatatatatatatatatatatatgtatatacatatgagggcatcggatcccatgacagatggttgtgagccaccatgtggttgctgggaattgaactcaggaccttggacctcaggacccctaatatcataaacaggaccttctggtactattctttttaagagaaccagactgagtttgagccatataaaagtgatattttatcaggtataaaacaagcaactaccaggtattcacatggttcagcctaatgcatatcattaagtatgcccatgcagatttggagaggcctaagaatattttattgtggggctggagatatggctcagcagttaagagcacttgctgatctcccagaggacctcaggttggttcccaggacactcactgtgtggtccacaacctcgaacttcagctccagaggatccaactgcctcttctagatcaggagcatctacacacatgcacatgcacacgcacacacacacacatactttaaaagacaaaaggaaatcttaaaacacacacacacacacacacacacacacacacacacaaatgtccaggccatggcactcaaccctcatcccaccacaaaccacaggtgagcaagtcataagccagagacctctgataggcagctcatgacccatgctgtccagctgagccctcatgcctacccatccctgagagtagagaaactggttgggaggatttggccactgactagcatccaccttatggtcccctggagcccatttaactccgatcccacagatgaaggtggtttctgtccccttcatctgtatgtgtctgctgttgccctgggatactttatcacatgcattacaaccccctccccctgctcttgagctctgtctcccagaagtggcctcactagataaacttcatatagcacctgtagaggtgtgacacagaagcatctcttacactcacagccttggccaaggcactcttccgccttcagaacctgaaagtgggacaggagctatatattgctcaggggtagagaattttcctgccatgcataaggtcctggcttccattcctagtacttcccaaaccaaacaaacaaaaacgccagaaagatggtccatctcccatcctttaccaggactccttcctcagtgtttcctgcttgtctgcccccaccaacacctgatctctccagccctgtgaccctctttctagcactacacagagtcagtcatatggaaacctactccccaaatcctgcagccctgagggccctctcgatttctgactcaaaaggtcctgccaactatctccatcagtcagcgacctctgccgagggccaagtgataaaggctcatagaattgccactatctctttagacaccagccttggccttcagaactgctccatagcacaggttttaagaaaggggaatgcgatggatcaggaagtggcaactaagattgaggagagtaaacatcacgaacattcccagcatgtgagaggcaaaaaggaaaaccaaccttatatgtaaggctgtgccatcttccacaggaaggctgaagatgtatatattttgctaatattcttttgagagccagaggacgcagtagaagggtttaaggagttggccatagaggaagccaagaagttggggaaagaaaaaagaattcatatctagaatgcaagaaagggggccctgggacgggcggtggtggcgcacgcctttaatcccagcacttgggaggcagagacaggcaaatttctgagtttgaggccagcctggtctacaaagtgagttccaggacagccaagactatacagagaaaccctgtctcggaaaaacaaaacaaaacaaaacaaaacaaaacaaaacaaaacaaaaacaaaaacaaaaacaaaaacaaaaacaagaaagggggccctgggttttcttccaactggttcataatcgggtgtatcaactatctatcgctgcatagcaaatgccctaaatcccagttcttaaaacactgagtcctgggctggagaaatggctcagaggttaagagcctgagttcaattcccagcaatcacatcacatggtggctcacaaccatctgtaatgggatctgatgccctcttctggtgtgtctgaagacaactacagtgtactcatatgcataaaaataaataaacaaatctaaaacaacaacaaaccactgagtcctttgctcatgatcctttggctgcactgaattcagacagctcatttcttctctttggtgtcaggtggacacactgaagtatttgatcagatctggggttggctgactgtcagcaagggtgatggcgatgcccaggaggcaagttcaggcacctttgcttcttggactcagggtccccagactaggtctcagcttgtccctacagtgagtttgtcatccacaccttggtcaaaaggtctcctctatccctacttagaaatgccttctctcccttgcaggcagtaccagtgacatcatgacgagtgaccacagccctgtctttgccacgtttgaagcaggagtcacatctcaattcgtctccaagaatggtaagcaatgggcaacgtcagcttttcttgttttcctcaaagacaaggggcctagggcatttgtcatctggttgcagctaccaattgtctgggttagatgtaggcctatcctttccttcccaaggcccatggtctgccctacctgatctcttcatgttcaggccacatcacaatcttagaccagaaaaccatatatatatagtgttttaagtaagtttcttctggataagcaagtgcttcaagtttcttccaattgtggtccaatttatttaattccatctaagtttaaaaacggactctaattaaagaaaatattagtccagtgttagtgtgggtgcatagtaggttgagatggtgaaagctatgtcggtgggatgtggatgaaggaggggagggaaactggaataaactcatgctttgagccacaggtatggttgcaaattgcccagcccagccctggttgctgagctaccctttctgcagcaatggctgggtgtgcctgttaacaagtccatccggtcctatcttaggtgaccaggaagccatgctgtcatctcctctacccacttctgtgcagcagataactgatttagggatgctgggtatgggtaacccaaatacagacagagaacagctgtctcactctatggcctctgtgctgagggatgtagacgaagctagacctctgtctttccaaccattggatcaaacatctgtggatctgatgtgacctcccttctccacccaagtcttcacacctgtccagtctccttcctacatctggccacctttatagcctttaggctccaccccttcctgctcatacctgtcccctcctttgagtttttcagcattgaaagagtagcctctatcacctccctctttgtcgggcttggtttccttctgctgggattcagcacacagcaggtgcttgaaaatattggtcaccagtttggagttttgatggaacatttgtctaagcagagtctggccaaggtgatgtggggtttagaaggagagaaagaagactaagaggcatggtggaggttgctgtcaagtggcttagaatttatatgaaaagatagaacacttgcttaaaaccagttcaagatcagtgtggtggtacacttaggtggtggttctagcccttaggaggcagaggcagcaggctcagaaattcagtttgagtctgactaaactacatagagtttgaggccagtttgggatacatataacaccctgtcctacaaaacaaaaacaatgataaaaaacagatgctgtgtaaataaaataaatgtcatgtaaattagcatagttataaccatgatagctgaaggccagtaagtcccagattccagagtagagactggttataactgtgtctgaggaagtaagagcaggtctccctagctgtggactcaaaacagcagatttgggtgttgagcctctaactctctgccagaacttgtggggaaatttggttgtattagtgcatgcctttaaccctagcagtggagcttaagaacttgatgcaggaagatcccaagtttgaggccagccttggctacatagtgaattctagacacacctgttctatatagcaaaaaggagagagggacagagggagaaggaggaagtaggaagggaaagacagacagacaacagagacaaagagacagacgggtggggggagggaggaagagaattaattagttgggagactagagtgaggttgagaggttgcccgaggccacacagattattgtcaaagctgggattgtagccatatcttgggtccttgctgtctttattcttcctgacctgcaccctacatgaacagcttgtgacagaccagagaatctgtggccatcaacagagatgctgagcttgggtcaggaagctttcctttaagacaaatctctccttccaacctggacatttcccatctgtgggccctcagagggagcctggcagagtgggtgcacagagaggaaaaccccagaaagaagctggaacctccatcttcagttcaggtacagtatacaaataaatcctagtctcccatgctccaggtcctggcactgtagatagccaagggcagatcgagtttcttgcatgctacgccacactgaagaccaagtcccagactaagttctacttggagttccactcaagctgcttagagagtaagtgccttgtgaactaccctttggggaggttctttcacataactacctttccattgataactggttgatccctcacccagttcattcgttttctctctctctctctctctctctctctctctctctctctctctctctctctctctctctctcataagaatccatggatggacccctcagtgggttcagcaaaggtggggtacctatggggctacccagtggctatgtggccctttccccacccctgtggtttgttcttctactgcctcagccctagagatagaggcagccactggtgtccatgtccctgtcccctaggtcccccctgtccctgacttgtccatcaacattctctaggcagaatccatttgagtttccaccaagctgggtttgagtttccattaagctgggtttgagtttccaccaagctgggttcttcttgctcagtgaaagccctgggcaaagctccagaatgctgaagatctaacagggcctaaatagctgctactgcctgcttgctgttgccctggccctccccttgccctttccatattgtggctcttgtgttgtggggctcactgtgactgggagcaaggtaaagatagacagcccatttcaggtggcaacgttgagcactcaccatgcttatgaaagatcctttctatagaacatctccagacatactgtctgagactcctatgcaagaaaactatatgctttctaagtcttcctgagatggttcaaagagtgatggggaaggataggaaccaaccttgcaaacagaggcacagactgtctgcttgtagagcccaggagagctaccagccttctaccccctcacacctttttgcaccttccttcaatgcatcccgaaccttcctgggattatgccaccttcaccacaacatcctgaagccccagctcagcagccagggtcaaaacagttccaactggaatgaggggctttgtgcgggtgtttttaataaactatcgagacatttgcatagatttctatggaaacagcatattaggaggcttaaattagaatttcaaaaggctgctatcttgagtgtcgtgggaagtgggacgagctgtcatgcttcccagtcttcctgcctgcggtgtgatgacttctttgttgcctgggatgttgcaaagaggagtcaaaaaggagggggaaatctctttgatttttcctaaaaataattagactgtgttggcaaatgaccatccttcaaacaaaaacagaatccccagaaagcctgactcctaattggtttggaggcctcagagacatgagaagggaaagacagtcctccgtggtcgaggaaggcgaccccttggagacttttctgcctggttctcactctgtggtctgcaaacccagcttcttcttctctaacaggttttgtcaagagtcaggaaggagagaatgaagagggaagtgaaggagagctggtggtacggtttggagagactcttcccaaggtaatccaggaagaaaatgtgcctggggcagagggctgcagcagtgcaggttagtacgcagcactgcaggctagtgtgcagctacagcagtgcaggcgagtattcagctgcagcagtgcaggtgagtattcagctgcagcagtgcaggcgagtgtgcagctacagcagtgcaggcgagtgtgcagctgcagtagtgcaggctagtgggcagcagtgcaggttggtggacagctgcagcagtgcaagttggtgtgcagcagtgcaggttagtgggcagctgcaacagtgcaagcgaatatgcagctgcagcagtgcaggttagtgtgcagctgcagctctgtaggctagtgtacagctgtagctggtctgcccagagtacagctacagcaggttcatccagactgtagccatcccagaccaaagccatagagaccagcttagacactgtagtcaagggacttagggtatgcttttcatcatggatggatgacctcaatattcctaagtggttttcagccctgctgtgcctactcccctttggttattacctacttagtgttcgcagcacttgagattgaatcgaggctcctgcaagcactaagcaagcacccaaccattgagctatgtccctgatttccctgaacactttttagcagcaggaagttgagggcagtgctgacatccaggagtctgtcactcaccaagccacatctctagcaaaccacagaatatttcaagcccaactggaggtttgtgacacatttactcagacagggagcaggcctatggtgtcttctctaatgcagccatagaccttggaggggtatgaggagagggatggtactgcaagtctcatttcagacctggctgctagaccttcccttggcctctgggatatgcaaagaatggaaggaagaatggaggcaatggaccagggacctggaagctcaactcaacccagtttaggaaatggggtggggcagggaagaaaactatctctcacttggctgagcaatggggtccacctgctctcttgggcatgaagagccaagacctgagtttgcttaaaagaacatattcagttccgttttactatgtatttatttattttatgaatgagtgctctatctgcatgtacatctgcatgccagaagagggtattaggttccattacagatggttgtgagccaccatgtggttgctgggaattgaatgcaggacctctggaagagcagccagtgctcttaaccactaagcctctctccagctctcctgatgcatgcaaacaccaagactgctgacccccatttacctgtcctgacctgccctcacctgtggactctaatctggtgtctcctctgcagctaaagcccattatctctgaccccgagtacttactggaccagcatatcctgatcagcattaaatcctctgacagtgacgagtcctatggtaagcatcccaggccagggacctcaccgtaccttagcctcagcctctgaagtgagggtgatggtggcttagcctttcaagaaagcctgtctgccaccacccattgtgccctgatgctgccacttcctctgtcccagagggacttttcctagtgggttaccatccctgctccttgtcacttctgggtgccacagccagtaacctcctgcttagccttgctgagtgagctgcaggtaccatgtaatttaatcttagccttaagaggcaggttctgttccccagtgagtaaataggtaaactgaggctcatagaggtcaagtgactccccccaggtcacacagatagtcctcaactgagctgtggcttgagcctgagtctgcctgtgccaccgtgtctctcgttagtccctagccagctcctcagacccaggtgcagaacattagcccgtccctgctgccaggtcagctttccagtctgtctccttttcctttcagacttttccttgtatagagattttagactgacttacactcttacctgtttatctgattttgtttatttcttgtactgtttcaaaccttgggaagtaagctttccctatagcaaccagattaacatttaataccatttcttagatggatttttctaggatttaatttttccctcagcattgatctgtatagagttctttttcctatgaggcttcagacatttgccccagattgctaaggcatcgattgaaagggtttgttggcaatccctgcccggccacttgcttcaaggagctgtctcccagtggtacagccatctctctgagcatgctctatatccgtctaatgccaagatgttcctattgatgctctctctcctttccccaggtgaaggctgcattgcccttcgcttggagaccacagaggctcagcatcctatctacacgcctctcacccaccatggggagatgactggccacttcaggggagagattaagctgcagacctcccagggcaagatgagggagaagctctatggtaggtcagccagcctcctcctggctttcccagaggccactgcactaaggacatgttctttctctcagcaaagctatcaagtatctatgatctgtgccttcagaatatagcgtggttagaaagtctctagggtagcgtggtgatcctggcactcaggaggctgaggcgagaagatcgtgagttcaaggctagtctgggtgacatactgagacgatgtctttaatgagaagaaaacaagtcatctcatttatctctcacaaccgccctgtgagtcaagtatggttatctctacttggcagatggagaaactggagctagatgactggcttaatgttgcaaactcaggctaagccaagttcatccagggccaagcaggagctggtagacatagtggttgcttgcctgtcatgaaaatagcaagtgtaggaacgcacaacatggggtgcaggggctccttatgccagggagggctctaagggccactgcttctgtgcatgtgactctcctgtgccccatgagagatctttcttttctctagcaggacagtgggttgaacagatagtgggtcattacctagcaaccacattccagaaataaagcccggggttcctttaaactgatttataggggttcccataaacagataagctctgcatgggaagtactcttatagggtaacctaatagctgttctaagtttgttctagtctgacaagaaatgtctgttttatccatggctaagctaacaaggtatttcttgaaataacaactgggtcataagtatctgttcatctccttctggacccatggcaggcatcatggcaggcatcatggcaggcaacagttgccaagcgtaaggattttgaagcctgaaaatcttgcttgatatacctctcccagtaaccactatttactgtccagctttggcctgcaaagagataccacattgccatttctaacccatgccgtggctaggatctcacatgttttctttcctaaatgttctcttccagactttgtgaagacagagcgggatgaatccagtggaatgaaatgcttgaagaacctcaccagccatgaccctatgaggcaatgggagccttctggcaggtagacgaagcttgctaagacttattacagctagctgggctgtgtgaaccagagtccaggagagggtaaagtggagttcaggaaagccaaggtcagaggagaagaaatgttgtggcccaggggacccactctcctacctcagtttcacatcctgagttcaaatcctcatctctgaaagatcaaagccttggagaacatttccatgtaggaagggctcagctcaactatcctttaatgaacacctgctatattcaggaagacacagtgtaaatacagtatagtccatgacctacagacttaacaccagccagtggaaggatgtgcagtgcaggaagaaggagctatagatgactgaacaattccagggcaaaagatacttctcctgcattcagggaggccaggaacataagtggtcaaaagtcaccacccatgacttcctagcttcatttctgttgctgtggcaaatgccctgattaaaaggtagcatggggagggaagggatcatttgacttcctcaagcccacattacagtctagtgttgtggagaagccagggcaggaacctgaagtatcacatcaacagtcaagagcacagagaataaacacatgcacccttgcttgttctcagttagttttcttcactcttacacagttcaaggcccagccaaggaaatggtgccacccacaatggactgagtcttcctacattaattaacaatgaagacaagtccctatagacatgcccacaagccaaacttatctagtcaattcttcataaagactctcttccctggtgatcctagattgtgtcaagagggcatttcaaaccaaccaacacagaggggtgtagcggggcatgcctttacttccagaactcaggacgtagacgggctgatcttgtgagttcacggccagcgttgtctacacagtgaattccaggacattcagagctacataatgagaccctatctcaagaaacaaaataaaacaaaacacagaaagactaaccaacacagtgactaacaggaaagacggattggagagtaacctgggcattccagtctatgttgtttcctgatatcagaaacacccctgtttcctgatgtgctcgtgttagtcctcctgagatggagcccattgtggaacaaggtttggtgtgtgactacaaaggttgcctcacctggctctggactgtctcctccagttttaacacagctgccctccttaccacctctagcctcaccctctgtgccgcacactcaccctgctggtctgatgccttccgtgggatgtgcaaaccaacatgtcccacgtgactctcccacagccccaggcccaccaggttctcattctgctctccccatgtgctgaccttgtcttcagtgaaccccacaacagggcagcctctatccctgaccacagggccttagcatggacctttgcctccacacagagcatcctcctctctttttctctcttctgtactccatataaattcaatccgcagacagccttgagtcaatcttctccagccatgttctacctggtgggtctccactctcctacttgtctcaacttgatctcttatttctctttgccatattatacacgtaccagaatgagtaaatgaattgttgtgcccaagaaataaaggtactgagaggctgagaagctgaggaacattttcctcaggctggtgccagccaagaagggaagaggcttcaggtgtgcctccacatcttcctacagggtccctgcatgtggtgtctccagcctcaatgagatgatcaatccaaactacattggtatggggccttttggacagcccctgcatgggaaatcaaccctgtccccagatcagcaactcacagcttggagttatgaccagctacccaaagactcctccctggggcctgggaggggggagggtcctccaacccctccctcccaaccacctctgtcgccaaagaagttttcatcttccacagccaaccgaggtccctgccccagggtgcaagaggcaaggtgagtgtcctctgaattgtgtgtgtgtgtctgtctgagtgtctgtgtttctgctcaaaagcatcccttgggggctcccaagggtgacggcctgaagagggcagagttgtagtaggttctgcccactactttggcttctgcctgtccaaaacagttggtgcaacttgattttaaaggggactgggttgggacttgccaagaaagccatcttcttataaaaactgcatttactcacaaagacatttctcaaaccaatgacaatctatgccatcctccccttcctggagttctcagtgggaaagaggtgaggatttctgaatagacaccattatctaccccaaatctctctctttccttaaaaacaagcattcatgttctcatatataactatctgggggctggagagatgactcagtggttgagagcactgactgctctttcggaggtcctgagttcaattcccagcaaccacatggtggctcacaaccatctgtagtgagatctgatgccctcttctggtgtgtctgagaagaatgacagtgcactcacatacataagtaagaaaataactatctggaccctcctgatggaaacatatattggaaactaggaacccaaatgaaggcagagctgtcatcctacagagggagccggccagaacaggtttaggagcaaggaccacacagcccagagatgaagtcctaagcagatatgggaaacattaggtgggaatcccattcctacaggatgatagatggccaagtgacatccagacctagttaacagtgacacagatgtgtctcctccccagctgccttgagcattttgtggtgacaccggctctcacattcagcctcttttctgagtgctgtttcttttcttcccttttcagaacttcagttccttagcagagctaactcatagtaatcagggacccgtgctggagcatcacccacagtgcggttctcctccagaccctctaagtcaaatgcctataccaggcctgcctggggattcctgtggagaggcactgacagtcctgtgccttagctgttagctgaactagctgaaaggtgggagggcaggtcccttagccagactgaagtctacttcctaggagcaagggagaaatcgccttggcatccctccccggaaatgaggatcaaggtagccatccagaaggtactgaggtacttgtttgacaaaggcagtctctttcgagaccccatgaagcagagctagagacagccacaaagaaagcacaagttcatggactcagaggttcccagagtggaagtcactgtgtgcttcacacggtgaacagagcttggtggaaacatgtcctctccagccccaggtgacagcataggggtagacagctatcagggagcgagctacaggactaggtggcaatagccacccatcccaggttccccaaggctgccccaactttagcatttaaaagtcccatctcctggaaaacactgcagtcctggtaagcttggaccaccaccatgtgaggtcaaggctgtgtgccaagaaaggagactctgctcaggccctagccggcatccatggtctcctgcacaagaactgactctgccccctggataagagcttggtttcctgttgcctatactcaaacttttttttttttttgactaaaactcgtgagtttaatctgtttttcctaacctccattaggatagcaggtgtaccctgaacactgctggaaggatcactttctagcttctatctcattggctccatcctctggcctctctatctctatagctgagtggatagccagatgccacacacatgcacaggcacgcacacgcacgcacatgggggggtggggagatggggggggagttgcttgcttctgtctaccacaatgccctcagaggccagaagaggacattggattcccaagaactgcagttacatacagttgtgagttactatgtagatgccaggagtcgaacctggtaactctggaagaagagcaaatactcttaatcacagaaccaactcttcagcccggcatggtgttgcatgcctttaatcccagcactcagcagacagtgaggcgggcggatctgtcagttctatatcaaccagggatacgtagtgagccctggtgtttaaaaagaaaaattctcaaggcagaatccatgtaaaaattattccctggaaaataagtaattcggaggcatttctgtggtgcatgttatatgatcactactgtagttagttccagagcattcttatcacccctaaatgaaacccagaggcatgaagcaactactccccatgtcctgacttctgaacatatttcctccccatctcctctccttttccccatggttccagacctggggatctgggaaaggtggaagctctgctccaggaggacctgctgctgacgaagcccgagatgtttgagaacccactgtatggatccgtgagttccttccctaagctggtgcccaggaaagagcaggagtctcccaagatgctgcggaaggagcccccgccctgtccagacccaggaatctcatcacccagcatcgtgctccccaaagcccaagaggtggagagtgtcaaggggacaagcaaacaggcccctgtgcctgtccttggccccacaccccggatccgctcctttacctgttcttcttctgctgagggcagaatgaccagtggggacaagagccaagggaagcccaaggcctcagccagttcccaagccccagtgccagtcaagaggcctgtcaagccttccaggtcagaaatgagccagcagacaacacccatcccagctccacggccacccctgccagtcaagagtcctgctgtcctgcagctgcaacattccaaaggcagagactaccgtgacaacacagaactcccccaccatggcaagcaccgccaagaggaggggctgcttggcaggactgccatgcaggtatgttgagctgtatgtatatgggtgtatatgcatatgtgtgcacgcatgcatctgtgtgtgtgtgtatgtgtatgtgtatcctgggatgtgctctgtgaagaagggctaaacctggggtttgtacctggcatctgttcctgttcctgactggtcctacagggcatctgcagtgattcttgagagcgacagagaggagctgttgacacctgtagagtaagggagtctaagtcatttatattttagtcttatcatccttaattagggtatgcacttaaaacattcctggtgttttagagacctcaggcagaatatccccttcctatagaatagatttattgtagagcaaaactaacaatgacttaaatacacacacacacagagttgagactggacatgatagtacaccgctataatcccagcacactgtacatctagagcaagaggattggaagttcaaggcaagcctgggctaaatggtgagacagacaacagcagcagtgagagagctaaggttatatagctcagtgggagagtgcacaggaagcctccagaaagcccttaggtgaatctccagtatcacaaggaacagagtctagaactagactgctggccattccattagcatacttattgcagcattgagggtcagacttggggccttacactaggcaagcactgtaccacagtcctgtgttggatttaaatcccaagtctccactgcacggctctatgcctttttaaactgtcaaaaagaaactactagtgccagccattgtgtagatttatggggagaaaaagattatgccatatactgttattgtttgtgatcatttttacagttatcactcattttgaaactatatacaacatatatctatctcataggtactgtgtttgtaatatatgcatgaattaacatataaggcacttaggacagtgtggctctttcacattaattagcctgacctaacttacagcactaggggtcaaatctggcaaggccctgatagccctttctgttctcatcttacagtgagctgctggtgatcggagcctggaggaacagcacaaagcagacctgcgcctctctcaggatgcctctctcaggatgcctcttggaggacctcctgctagctcttcttgcctagcttcaagtcccaggctgtgtattttttttcaggaaacggcctcacttctctgtggtccaagaagtgtgctgctggctgccacactgtgcggcagatgctaaagctggatgacaaacgcacgccatacagacagcagacagcggcactgggtctcagaacttggattcctgggccttcttccagtcgccgttttaaagaaaggaactaacggagctgctcatccgagggtgaagatataaataataatattattaataataataacagtcaggtgccatgtgctgtgttaagtgctttatgaacatttgtcgggctggcctccagtgctgaggtgccagtcagcctgaaccctatgcccaggcccactaatcccaaatggtgggtcctgagatgtttttaaaaagcattaaagaaaaccatcggtctcttagagctaaccggccgggctctactgcagggacccgaacagtctgcatggctaagtggcacaaggagcctggccctgtccagcttcagagatccaagctgctttttgctggggttctgtcacaggcctgatcctcttggtttttatggggtttcaagtctgccagagtcagaaatcagctctaactcgccagtgaagagatctggccttaacttaagccagccacgtcaggcccctgctgagcctatggaccaataaatactccccgtgccactggaggtgggcagctatcaccataccctgagttgggccaagcccaccccacccctaccctgcaacatttctgatgtactgaggaagagtctccaccatagtccccaagggctgagttctccagcctgctatcagggaaggtgagcattggtcccaggctctcaaaatagtgcagcctcttcttcccaagctctggggtgcaccctgtgtccttggttaccaggagactagggttgtgatatcttttcttgtcttgctttttgatatatcaggattaatgtaggaaaccagacctagattattcaggagagtaggtatatcccctgtgtttccca

SEQ ID NO: 2 - DNA, 11928 bp, Mus musculus
gcttcccgaggagcacctgaaagccatccaggattatctgagcactcagctcctcctggattccgactttttgaagacgggctccagcaacctccctcacctgaagaagctgatgtcactgctctgcaaggagctccatgggtaacggagagccctgagagaggggtgggggagcttcatgggtaatgggagcccctacccacccaggaggatggccacagcaagagaaagtgctcattagagtgaccctgggtctcctctctgtccagatgtctctgcagcactcacagtaattggcccaggtggagtctggaatgttccaggcttgttggaagctcttgctctcatagaatctgagctctaactgagctgggaaagttgatcatttgtttattccttttagggtattgggggggcacggatgtatgcatgctggtatgtatgcatgctggggatgtagggcctcatgtgtgctaaatacatgtgcctattctgtaatgttttcttgtttgtttttaactcaaattaattagaggcagtttctctgtttaaaaaaaaaaggtggaagacactgccatctcatttgtgttgggacttgagatatatatatatatatatatatatatatatatatatatataatttatttatttatttatttgtgtatgtgtgtataagaaagcgggggacactcatgtgccatggtgtgtatggggacacacgtgtcctgtgggggaacattgtgtcaggggaacaagcatgtgctgagggtggggggatatatatgtgccactgtggtgatttggttgggggagacatgtatgtgctatgatacctgtgtgtaggacagaggacagactcacacaggagccttcttacctgctgagctgtctcagcagtccaagccctccaggctgtagccactcttcctccttgctacagtcccacatccagccaacacataaaggctctggcaggaaataaattaaatttgctttgtgtgtgggtgctgacggctcaagtcttccggtgatggtaatgtttaaagcaagccaacatcagttctccagtgccgaagttattgaatgactgaccaatgggtaacacttaggatttttaaaaattattttgattgtacattttgaattacattaatcttaaaataaactacaaacataaacacactgtggttggagctgggcatagtggcacatgcctttaattccagaacttgggaggcagaggctggcatatctctatgactttgaggccagcctggtctatataatgagttccaggacagagagatcctgtctcacaaacagagaaaaccccataactatacttttattgtactggttggtctacagtgtaaagaattggcaaagaatatgaaagattacactgggaagaaactgaaagccatccagagtaatgaagcaaacccctcacaaatgtgggaaacatttcacatgggtgagggcttgcagccagcctgtgctaatttatgttttggtacgtgagcacttagagcagtttccagttctctggtgctctaccaatcttagcttggttaatattcagggatgagtcttccatcaccggtaatctaatttgccattctatttaaaggctcttaaaggcacaggcagtgcattgggtaaatgtggcaaataatttctttgacaaatcgaccaattgtcagattggcctgctagtcatttgtttcaatgagaactgttttttctcaaaggatgctcttgtacaccggctagaagcagggctgtcatttttataggtctctgtggtattttgttgttgttgttctgttgttgcaaatcatttatcactgagggaaaatacacacaaagggccctttctttaaaagtatacatgtatcattttgtgacagctcataagaagctgtttttttctgcctggacacaggtcctgacctgtgctgtgtccttgctaagctttgtcagacccttccacagctccccccaacaacgagttccccagtacctgcctcacctcatcactatggtgacagcagcctctgatgcgcctgactctctggcacattatggcagtgttaaaagcttccatctctctctttgctgaataatgaacctcaggttgttcaagagaccggaatgttcttcacctgcctgcacacatctcttcactttcttttatagatcaggtagggactgggcgtgtagatggaacaaactgttttccgttccccagccatctctgcaggtgcactccacataaatcaagtgttaaaagtgctttgattaaacaggacaggcgcgttcttgagttcatctgttcacatactgtctggcaagcgctgactgagggtctcctctgtaccctgttctgagaactaacaaaagacgaatcaacatacagaaaactgttatttagtgactgattaaactaacgaaggcatgggctggagaaataactcagcagttaagaacatttgctgatcttgcagaggacctgggttgggttcctagcacacacagggacagttccagtcccggtgtgtccttttctcacttctgtggacacaagttttacacatagtgcacacacatacactcacatatataaaacagaacatttaaaagtatgtttaaataacggaatcatttatataggttttcatttacataggtaaataggcaaaaatctgcattttattgtttctaagttttaatttatttttctctgtgtgtacatacgcatgcctccttatctgtatgtgcgtgcactgtgtgcatgcatgaacccacagagaccagaagagtaccacagattctctggagctggagtgattgataggctgttgggagccactccacatggggattcagagttgaacttcgttctctgcaagaacagccagctcttaactgatggcttttacctccagccaccttttcctcatttttaaaatttccttccttcctttttgagacagggtctcaatacttagctcatcccaacttgaccccactcttctcttgccttagtcaccacaatgtttagtttataagcatgcgtcactatgcccggctttaaataaactcacccataatcccagcactgaagtagacaaaagggaggatcgatggggctgactggccacaagccgtgcttcaagttcaatgaagaccctgtctcaagggaataaggcacagaggatagagccatacgcctgacctcctcctctggcctctacccaggcacatgtgcatacacacaccacacacacacacacacacacacacacacacacacacagagagagagagagagagagagagagagagagagagagagagagaaactttttcctctttttttttaaaaatattatttatttcatgtatatgagtacactgttgctgtcttcagacaccccaaaagagggcatcagatcccattacaaatggttgtgagccaccatgtggttgctgggaattgaactcaggacctctggaaaagcagtcagtgctcttaaccatctcttcagccccacaaagaaacttttaatgagcaaataattgcttccaagtaaatactactaatatatttctaaccatactatacaaggaattattaaagaacggataataggagaataaaaaattataagtcactttataatgctatctaatccatctagaacaaaaacactgtaataatgcaaaagagcgcagtgcctagattaaataaataaaatgcagaccaataagtaaactttatagcagcacatggaaatgacgaaattcctaacaaaaagctcaagatgggcagtttatttaaagtgaaatacaggagaaataaagcacagaaagatactcaaaggcatagaagttaacataggggggctggcgagatggctcaacaggtaagagcacccgactgctcttccaaaggtcctgagttcaaatcccagcaaccacatggtggctcacaaccatccgtaatgagatttgactccctcttctggtgtgtctgaagacagctacagtgtacttaacatataataaataaataaaactttaaaaaaaaaattaaagaagttaacatagaagcccactcaggaccccactcagtcctagagtatgacattattatggacattaaaaagagaaaattcagcagtagtgtgcatgcactgcatatatacacaaatccttgagtttcataccaaatgcctttagaccacttgtggctctgcaaacctgtaatcctagcacttgtgaaaaggtcagctcaggaactttgggaaggtcatgaaactcttgcccctccagaagggagaggctaattaacatttctcagaccacagggcgggaaccgacctgcgggtggggacagactgttgcccatttccagactagggaagtccttgtcacctcattccctaaagaccaatcaatttaaagggtgcactgttccgccaatcatattgtgcctagttgctgatgctctattctgcccttagaaaccgtataaaaactagcgaaggggtaccaggggtaaccccctctccttcaggtctgggacaatcccactacactggaacaataaattcctcttgctttttgcattgatcacagctccacttcgtggtaagctaagactccctggagtcttacattggcaaatgcaggcaaaagaatccgaaactcaaggtcatctaaaactacatagcaagcatgctgctagcctgggctccatgagaccctgggggaggggcagagggagaccgttcagaagacagtcaagatgttgcagcagcacaggcagcctggccaccagtgctgtcaccagacatgttaatgttggaataaagcctcaatcatgactctcccagttttataattggaaataagaaaggaaagactataggaacaactgtgttcagaacactatttataatagcaaagatctcagagtaacccaaacttctagacattgatttgggaagatctcttggcagcttattttgaaaactttacaatgttaaatatgtaaaaacaaggacagttttgttttttgttttgttttgttttgttttagggatatatattcatatatgtatatgaatgaaaacccaaacttaaaattccccactatgctttaaaggctttctgacaataacagaaagagaaatagagaatccataaaaactagttctgaaactatcaataggcttgacactctttagctgccaggagagctgaatctgaacacagggaaccccacccagcaccccaaatttggattattgttttattttatctttcccctacccccaagacagggtttctctgtgtggtcctggctgtcctggaactcggagatcctctgcctctctgcctctctgcctctctctctctctgcctctctctgcctctctctctctctctctctgcctctctctctctctctgcccctcgctgcctctctctgcctctctctgcccctctctgcccctctctgcccctctctcttcccctctctgcctctctctctgcccctctctgcctctctgcctctgcctcctgagtgctgggatttaaaggcatcagccatcacttccagcttcctttatcattttaaaaagaatttcctatgtgactactgtatttaaatcaccacacggccaatactcccccccaactcctcccaaatcccctctacccactcaaattcttatcttgtattctttatcattattatacatatgtgtatatatgtgtgtgtgtatatatatatatactatatactgctaatgagtaacatttagtgttattcattgttgcatgttttcaatgtgctttccaggaggctggggggatggctcagtgggcaaaattctagctgcacaagcctaaggaccagggttcagatccccaatataaaggctggctggacatggtggcttgcctatgatactagcatgcttgctggaagcaaagacagggaatccctggagacttagaatctcagaagtgatctgggctggacagactagctgaactggccagctctgggttcatcaagaaaccctacctccataacataaagtgtgatggagaaaggcacctaatgtcaacctcaaacccctacctgcatgtgcacacacatacatccacaccacacacacacacacacacacacacacacaccacacacacacacacacaaataaataagtaaataaataaaatatttagctctccagaccaaatcttggtgaaacccatgcatttgcatttgtgtgtgtcctacaaacactgaaggttaagaagcatgctccttagtaattttatagcagtttgcgtttccagattgaaaacagattctataggctacacagtgctaaatggattatgctcagatacagattgaaaaggatacagattgaaaagggtcggggtctgggccaggatgacgggccaactgatctttgccggggcttgtccttcagggaagggttacaggattcaccactggggtgtggcctatctgctgttaggacctgaattgcctggagtgtttctagttcccactagttgttgaactttaccttgaacctctgctcccagggaagtcatcaggactctgccatccctggagtctctgcagaggttgtttgaccaacagctctccccaggccttcgcccacgacctcaggtaaggggtttggatttggaaagatgcaattgctataggagggactctgaaggcagacagacgcaccgcctcctcacgttggctagtctaatataaacatcgcggtggatggtgaggatagactccatgcccttttgtgaaggcatttcctggcatcagctcctgacttcagacagtttcacccatcagacaaattgcctggtgttggaggaggaggtgagcagggccattcccatcatttctcctcagaaatggaaaggcaaggaaaacatgaggttcttcagacacttaatccctgggactgcaaaatggtggtgcccctcctccacagctgctcacggcggggcaggagatgagggccaaatgaagcatagatctagctattttttttttagtgccttcagtaaatttaaaatcaaataagggaagggacctagatctttatgttatggcattgttaaaagtgagaacttgtagccagggtgtggtggcgcacacctttaatcccagcacttgggaggcagaggcaggcggatttctgagtttgaggccagcctggtctacagagtgagttccaggacagccagggctacacaggtttctctatacagagaaacctgtctcgaaaaaccaagaaaaaaaagaaagaaaaagaaagaaagaaagaaagaaagaaagaaaggaaggaaggaaggaaggaaggaagaaagaggacaacatggtctaggggtcagagagcagaatctccaaaaacaccaacaatgcctgctgtaaatgtatgtcgttgatttggggatgttggcctccagctcaccatttcctgccttagcctccaaagtgctaggattataggcttgagccaacacatctggcttacgcctattgtgtgtggaaggggagtgctgagtgtgctcctgtgtttggtacttatatatgaatatatgtatatacgcatgtacgcatacttgcatgtgaaggccagaggccaatgtcagctgctctcatcttatcctttttattacattgtatttatttgtttgtttgtttgtttgtttcatcgtacgcatgcagccactcatgagcatgtaacagcacaggtatgaaggtagacttgcaggagtcagttctctccttctgtcacttgagttccaggaccacactccagcccccaggcctgggctgtaagagagccatcttactggtcctctactttgtcttctgagatagcatctagactcacggaacctggagctcatctagatttacattggctggccagctgatgcattttaaggtcaaatcttcattccatccctaccccacttccactcccagtgctggagttcgggacacctgccaccaagcccagtttttcctggatgcagaagctccaaactcaggttcccatgttcgcatggcaggcacattttcagttaagccttccccccagctcctttaccctggtctctgaatgggggggaggctataaatcaggctgctctcagacattaggtaggaaatagaccatatacatgaggaaagatattcacctgccccatgggtaccaggaagtgatgtccaactcctcttttgcttatcaggagaaatgctgactactacctctggtaattttgatgttgggaggaacagggacattcataggaccccattcctcgctggtgagagtggagacaggttttctgaagggcaggagatctgtgtagaaaagatggatgctgttttctgaagggaaatggaggtagagtcgacctgggagagaggggaggtgggggatggttgggaggaatgaaaggaagagagacggcagttgggatgtattgtataagagaagaatcagaaagaaaaaaagaaaagctacctgcacccttcaagtgttcctctgtgtgggaggctgtctcagggactacatgggcaccgagaggcatcagtgagggtaggtacttgatgttgtgtccctgaaaacaaggacaggaaatctgctgcatggcctaagatggcaaaatgtggcacaatcaagtaaggcccaggattctgtctgtggtgcagacctgctgtagaatgagctcccagcattcccacttgctgtgtggagacagcatgttgcagagccatgtgaggatgagggtccaggccaggaggatgtcaacccaccaccatgtagccagtgggctgggggagcttgggcccaccaaggagcttgagcagactgacagtgggttatgtacacaagtgggcgtgtcacacaaccgtgcaacacagagaaaatccctgtgatgacaacttctaaaccaccctgaggcaaaaggagtagacaggggattagagcctagcatattggagtcgagtggccatgcagctcttggaagcgtgaggaaggaaatttcctggaaggataggttgtcttcctagcagcctcgtcaatagatgtcaatgtatgaggtagtacctgctacaatcctgcttcttcagaagactgaggcagggggattacttgaacccagaagttctaggccagtatggacaacatagcaagagtctgatttaaaaaaaaaaaaaagtaaagagggaaaccaaataggtgacgtgccacactagtgtcttcctgctccaagggtcctgggcacatgagcttgcttagtgccagaaaagtcagaggaggagagggcagacagagaccctcgtctccacctcctttgactgactaatggggctggatataatctgttttacaaaaggacagcttttcagagctgtttctatctaaggttgctctgaatagccatctcgaaatatgccagagaagaatatttagaggcggcctattttggtctcccacaaagatttcacagggaaaatgtatttgtgttctatttatacaactaaaaatatgcatcagcccggggaaactggctctttgctgcctttgaagtgaaggatgggttaatttctaagaaagtaaaagcaaatgtagtgcaggcaccacggatgctgccagacaccagcgtttaagtggcttgaatggaagagcacaccccaagtatttgaagtaggtgaggagagagaccaggtctccagagttgggcctgcagtggccagggtaagtccaaggggcagtatgacgcagaacagagtggcaacctctaccagtagtagaattcaggtctcattcctaacctcccataaagcagagaatattgcactctctttctctctctctctctctctctctctctctctctctctctctctctctctctaactcacagagatcctcctgcctctgcttcctcattgctggaaataaaagcatgtgccaccacatgcagctctccttgtttggagagagaaaggcagacagacagacagacagacagacagacagagtataatgtgtgcagatgtccacagaactccgaagagggtgttggacccccagaaaccggagttctaggcagttgtaagctgtccagtgggtgttgcgaactgaactcaagtcctctggaaaactggaaagtactcttaaccactaagctatctgttagtcccccaagaatgtcttatcttgataggccttcagatctcacagtccagggggctgacctgctacaaggggtagaggaaagaaaactggctccaggcctgagttcagagtgacatctccagagttcttccttcccttgccgtgtagtaatttcctaccctacacgaggagaaaaggaacagatatgtccagtatgcctggcatcttgaaagggcactgactcttgccgctgtagagcccctttccttggggagtgcagagaagtgctgctagagaggttcaaaagaaacacaacagtcaaaatagttgctggccaagggagggcatgtgccctgtatcgggcctgcaaagccctttcaacagtgtagccaaggccatgtttgacagtacaagcctgtaagcccatgcataagcaagggctgggataaagggctactgttcaagttagttatatacacatcaagtttgttcatcttacatccttaggtaagggtgggtagcttgttagctccacctctccagcaggaaagcatgtccacggtaaggtagatactgtagagtttagctttgttttgacctttctttcttcgttgcttgggattgtacttgggaccttcactcatgcagcccatgtttcagccttgcataaatctataaatatatggttggagtcaggtccagcatatgtgggtaccagtcccagtctggtatagcaatttattagctatttgtctttagacaagtaactatagggttctgagctgtaatggccaccaaacaggtcctttaagtatctttattggccaaacaccagggttaaaacactgaacacagtatggtggtttcaactgtcatggagtccacattctagcaggggaattttacacacaactcacaaatgatacagaccacagcagggtgatttgacagagacaacgaagggcttgcctgagctgcagtagtccgggagcagctcttgatgggatctgagatctagatggcagagcgcccttcctagatgaaatccaagcctctggttactcctcagcaagagtagctggaggaagaccagaagtgaggaggaaaaggctcagagtggagcatctaagcctgcagcacctccagagacaggctttggagcttgcctgtggttcagcctactgtgggaagagaccagtgagggttttaagctgtggggatgggtatattttggaaggcgaacaagaacaccagcagctcaggcaatgtaggagggcgggaaggtgatggatgatcgaggacggccccagagattttaaggactgtgtaggtggggtgagaagcactgtgggtgaggtggagttagagggaagaagtgaagatctatttttttggccatgaggaatttggggtgcacacacacacatacatacacaccccatcatgtccttgtctacagacggggtgataatggtactggaaggtagaaggtggaaagaggaccagcaaggaaggtatccagtgcccaccccacagctcaccctcagacagaccttgttctcattttcattacccaggtgcccggagaggccagtcccatcaccatggttgccaaactcagccaattgacaagtctgctgtcttccattgaagataag

SEQ ID NO: 3 - DNA, 560 bp, Mus musculus
ctctgggttcatcaagaaaccctacctccataacataaagtgtgatggagaaaggcacctaatgtcaacctcaaacccctacctgcatgtgcacacacatacatccacaccacacacacacacacacacacacacacacaccacacacacacacacacaaataaataagtaaataaataaaatatttagctctccagaccaaatcttggtgaaacccatgcatttgcatttgtgtgtgtcctacaaacactgaaggttaagaagcatgctccttagtaattttatagcagtttgcgtttccagattgaaaacagattctataggctacacagtgctaaatggattatgctcagatacagattgaaaaggatacagattgaaaagggtcggggtctgggccaggatgacgggccaactatctttgcccgggcttgtccttcagggaagggttacaggattcaccactggggtgtggcctatctgctgttaggacctgaattgcctggagtgtttctagttcccactagttgttgaactttaccttgaacctctgctcccag

SEQ ID NO: 4 - DNA, 560 bp, Homo sapiens
agcccgaggattggagttgtcaatggatgtgacaatgggaaaatccgtctgagcctgcatttgggctgctaggaggggatttgcatcagaatccacagatcaccagcactgggcagccctaatatttaaaatgcagattctagactcaatcaggcgggagcccagaaatttgcattgttaacacctgtgtgtgtgtgtgtgtgtgtgtgtgtgtgtgtgtgtgttttataaacacagaaggttgggaaccatggataactaagtgaagtcattttgtcactcagatttgaattttctacaggctatagagtgcagtttggctaaagcaaaacctaggtacagtcaggactacacaattccagttcgctgtgggttgggaagggatgggtgggccagtgctggcaagccttgatctttgcccgggcttgtccttctggggagaattacctgcttctgctggactgagggtgccctcatctctggctagagcccgtgctgccatggaagactctttccggtgcccactaatccttgatgttcaccttgtcccctgcccccag

SEQ ID NO: 5 - DNA, 11539 bp, Mus musculus
  modified_base (1847): N = A, C, G OR T/U
gagctctaactgagctgggaaagttgatcatttgtttattccttttagggtattgggggggcacggatgtatgcatgctggtatgtatgcatgctggggatgtagggcctcatgtgtgctaaatacatgtgcctattctgtaatgttttcttgtttgtttttaactcaaattaattagaggcagtttctctgttaaaaaaaaaaaggtggaagacactgccatctcatttgtgttgggacttgagatatatatatatatatatatatatatatatatatatatataatttatttatttatttatttgtgtatgtgtgtataagaaagcgggggacactcatgtgccatggtgtgtatggggacacacgtgtcctgtgggggaacattgtgtcaggggaacaaacatgtgctgagggtggggggatatatatgtgccactgtggtgatttggttgggggagacatgtatgtgctatgatacctgtgtgtaggacagaggacacactcacacaggagccttcttacctgctgagctgtctcaagcagtccaagmcctccatggctgtagccactcttcctccttgctacagtcccacatccasccaacacataaaggctctggcaggaaataaattaaatytgctttgtgtgtgggtgctgacggctcaartcttccggtgatggtaatgtttaaarcaarccaamatcagttctccagtgccgaagttattgaatgactgaccaatgggtaacacttaggatttttaaaaattattttgattgtacattttgaattacattaatcttaaaataaactacaaacataaacacactgtggttggagctgggcatagtggcacatgcctttaattccagaacttgggaggcagaggctggcatatctctatgactttgaggccagcctggtctatataatgagttccaggacagagagatcctgtctcacaaacagagaaaaccccataactatacttttattgtactggttggtctacagtgtaaagaattggcaaagaatatgaaagattacactgggaagaaactgaaagccatccagagtaatgaagcaaacccctcacaaatgtgggaaacatttcacatgggtgagggcttgcagccagcctgtgctaatttatgttttggtacgtgagcacttagagcagtttccagttctctggtgctctaccaatcttagcttggttaatattcagggatgagtcttccatcaccggtaatctaatttgccattctatttaaaggctcttaaaggcacaggcagtgcattgggtaaatgtggcaaataatttctttgacaaatcgaccaattgtcagattggcctgctagtcatttgtttcaatgagaactgttttttctcaaaggatgctcttgtacaccggctagaagcagggctgtcatttttataggtctctgtggtattttgttgttgttgttctgttgttgcaaatcatttatcactgagggaaaatacacacaaagggccctttctttaaaagtatacatgtatcattttgtgacagctcataagaagctgtttttttctgcctggacacaggtcctgacctgtgctgtgtccttgctaagctttgtcagacccttccacagctccccccaacaacgagttccccagtacctgcctcacctcatcactatggtgacagcagcctctgatgcgctgactctctggcacattatggcagtgttaaaagcttccatctctctctttgctgaataatgaacctcaggttgttcaagagaccggaatgttcttcacctgcctgcacacatctcttcactttcttttatanatcaggtagggactgggcgtgtagatggaacaaactgttttccgttccccagccatctctgcaggtgcactccacataaatcaagtgttaaaagtgctttgattaaacaggacaggcgcgttcttgagttcatctgttcacatactgtctggcaagcgctgactgagggtctcctctgtaccctgttctgagaactaacaaaagacgaatcaacatacagaaaactgttatttagtgactgattaaactaacgaaggcatgggctggagaaataactcagcagttaagaacatttgctgatcttgcagaggacctgggttgggttcctagcacacacagggacagttccagtcccggtgtgtccttttctcacttctgtggacacaagttttacacatagtgcacacacatacactcacatatataaaacagaacatttaaaagtatgtttaaataacggaatcatttatataggttttcatttacataggtaaataggcaaaaatctgcattttattgtttctaagttttaatttatttttctctgtgtgtacatacgcatgcctccttatctgtatgtgcgtgcactgtgtgcatgcatgaacccacagagaccagaagagtaccacagattctctggagctggagtgattgataggctgttgggagccactccacatggggattcagagttgaacttcgttctctgcaagaacagccagctcttaactgatggcttttacctccagccaccttttcctcatttttaaaatttccttccttcctttttgagacagggtctcaatacttagctcatcccaacttgaccccactcttctcttgccttagtcaccacaatgtttagtttataagcatgcgtcactatgcccggctttaaataaactcacccataatcccagcactgaagtagacaaaagggaggatcgatggggctgactggccacaagccgtgcttcaagttcaatgaagaccctgtctcaagggaataaggcacagaggatagagccatacgcctgacctcctcctctggcctctacccaggcacatgtgcatacacacaccacacacacacacacacacacacacacacacacagagagagagagagagagagagagagagagagagagagagagagagaaactttttcctctttttttttaaaaatattatttatttcatgtatatgagtacactgttgctgtcttcagacaccccaaaagaaggcatcagatcccattgacaaatggttgtgagccaccatgtggttgctgggaattgaactcaggacctctggaaaagcagtcagtgctcttaaccatctcttcagccccacaaagaaacttttaatgagcaaataattgcttccaagtaaatactactaatatatttctaaccatactatacaaggaattattaaagaacggataataggagaataaaaaattataagtcactttataatgctatctaatccatctagaacaaaaacactgtaataatgcaaaagagcgcagtgcctagattaaataaataaaatgcagaccaataagtaaactttatagcagcacatggaaatgacgaaattcctaacaaaaagctcaagatgggcagtttatttaaagtgaaatacaggagaaataaagcacagaaagatactcaaaggcatagaagttaacataggggggctggcgagatggctctacaggtaagagcacccgactgctcttccaaaggtcctgagttcaaatcccagcaaccacatggtggctcacaaccatccgtaatgagatttgactccctcttctggtgtgtctgaagacagctacagtgtacttaacatataataaataaataaaactttaaaaaaaaaattaaagaagttaacatagaagcccactcaggaccccactcagtcctagagtatgacattattatggacattaaaaagagaaaattcagcagtagtgtgcatgcactgcatatatacacaaatccttgagtttcataccaaatgcctttagaccacttgtggctctgcaaacctgtaatcctagcacttgtgaaaaggtcagctcaggaactttgggaaggtcatgaaactcttgcccctccagaagggagaggctaattaacatttctcagaccacagggcgggaaccgacctgcgggtggggacagactgttgcccatttccagactagggaagtccttgtcacctcattccctaaagaccaatcaatttaaagggtgcactgttccgccaatcatattgtgcctagttgctgatgctctattctgcccttagaaaccgtataaaaactagcgaaggggtaccaggggtaaccccctctccttcaggtctgggacaatcccactacactggaacaataatttcctctggctttttgcattgatcacagctccacttcgtggtaagttaagactccctggagtcttacattggcaaatgcaggcaaaagaatccgaaactcaaggtcatctaaaactacatagcaagcatgctgctagcctgggctccatgagaccctgggggaggggcagagggagaccgttcagaagacagtcaagatgttgcagcagcacaggcagcctggccaccagtgctgtcaccagacatgttaatgttggaataaagcctcaatcatgactctcccagttttataattggaaataagaaaggaaagactataggaacaactgtgttcagaacactatttataatagcaaagatctcagagtaacccaaacttctagacattgatttgggaagatctcttggcagcttattttgaaaactttacaatgttaaatatgtaaaaacaaggacagttttgttttttgttttgttttgttttgttttagggatatatattcatatatgtatatgaatgaaaacccaaacttaaaattccccactatgctttaaaggctttctgacaataacagaaagagaaatagagaatccataaaaactagttctgaaactatcaataggcttgacactctttagctgccaggagagctgaatctgaacacagggaaccccacccagcaccccaaatttggattattgttttattttatctttcccctacccccaagacagggtttctctgcgtggtcctggctgtcctggaactcggagatcctctgcctctctgcctctctgcctctctctctctctgcctctctctgcctctctctctctctctctctgcctctctctctctctctgcccctcgctgcctctctctgcctctctctgcccctctctgcccctctctgcccctctctcttcccctctctgcctctctctctgcccctctctgcctctctgcctctgcctcctgagtgctgggatttaaaggcatcagccatcacttccagcttcctttatcattttaaaaagaatttcctatgtgactactgtatttaaatcaccacacggccaatactcccccccaactcctcccaaatcccctctacccactcaaattcttatcttgtattctttatcattattatacatatgtgtatatatgtgtgtgtgtatatatatatatactatatactgctaatgagtaacatttagtgttattcattgttgcatgttttcaatgtgctttccaggaggctggggggatggctcagtgggcaaaattctagctgcacaagcctaaggaccagggttcagatccccaatataaaggctggctggacatggtggcttgcctatgatactagcatgcttgctggaagcaaagacagggaatccctggagacttagaatctcagaagtgatctgggctggacagactagctgaactggccagctctgggttcatcaagaaaccctacctccataacataaagtgtgatggagaaaggcacctaatgtcaacctcaaacccctacctgcatgtgcacacacatacatccacaccacacacacacacacacacacacacacacaccacacacacacacacacaaataaataagtaaataaataaaatatttagctctccagaccaaatcttggtgaaacccatgcatttgcatttgtgtgtgtcctacaaacactgaaggttaagaagcatgctccttagtaattttatagcagtttgcgtttccagattgaaaacagattctataggctacacagtgctaaatggattatgctcagatacagattgaaaaggatacagattgaaaagggtcggggtctgggccaggatgacgggccaactatctttgcccgggcttgtccttcagggaagggttacaggattcaccactggggtgtggcctatctgctgttaggacctgaattgcctggagtgtttctagttcccactagttgttgaactttaccttgaacctctgctcccagggaagtcatcaggactctgccatccctggagtctctgcagaggttgtttgaccaacagctttccccaggccttcgcccacgacctcaggtaaggggtttggatttggaaagatgcaattgctataggagggactctgaaggcagacagacgcaccgcctcctcacgttggctagtctaatataaacatcgcggtggatggtgaggatagactccatgcccttttgtgaaggcatttcctggcatcagctcctgacttcagagtttcacccatcagacaaattgcctggtgttggaggaggaggtgagcagggccattcccatcatttctcctcagaaatggaaaggcaaggaaaacatgaggttcttcagacacttaatccctgggactgcaaaatggtggtgcccctcctccacagctgctcacggcggggcaggagatgagggccgaatgaagcatagatctagctattttttttttagtgccttcagtaaatttaaaatcaaataagggaagggacctagatctttatgttatggcattgttaaaagtgagaacttgtagccagggtgtggtggcgcacacctttaatcccagcacttgggaggcagaggcaggcggatttctgagtttgaggccagcctggtctacagagtgagttccaggacagccagggctacacaggtttctctatacagagaaacctgtctcgaaaaaccaagaaaaaaaagaaagaaaaagaaagaaagaaagaaagaaagaaagaaagaaagaaagaagaaagaaaggaaggaaggaaggaaggaaggaagaaagaggacaacatggtctaggggtcagagagcagatctccaaaaacaccaacaatgccttgctgtaaatgtatgtcgttgatttggggatgttggcctccagctcaccatttcctgccttagcttccaaagtgctaggattataggcttgagccaacacatctggcttacgcttattgtgtgtggaaggggagtgctgagtgtgctcctgtgtttggtacttatatatgaatatatgtatatacgcatgtacgcatacttgcatgtgaaggccagaggccaatgtcagctgctctcatcttatcctttttattacattgtatttatttgtttgtttgtttgtttgtttcatcgtacgcatgcagccactcatgagcatgtaacagcacaggtatgaaggtagacttgcaggagtcagttctctccttctgtcacttgagttccaggaccacactccagcccccaggcctgggctgtaagagagccatcttactggtcctctactttgtcttctgagatagcatctagactcacggaacctggagctcatctagatttacattggctggccagctgatgcattttaaggtcaaatcttcattccatccctaccccacttccactcccagtgctggagttcgggacacctgccaccaagcccagtttttcctggatgcagaagctccaaactcaggttcccatgttcgcatggcaggcacattttcagttaagccttccccccagctcctttaccctggtctctgaatgggggggaggctataaatcaggctgctctcagacattaggtaggaaatagaccatatacatgaggaaagatattcacctgccccatgggtaccaggaagtgatgtccaactcctcttttgcttatcaggagaaatgctgactactacctctggtaattttgatgttgggaggaacagggacattcataggaccccattcctcgctggtgagagtggagacaggttttctgaagggcaggagatctgtgtagaaaagatggatgctgttttctgaagggaaatggaggtagagtcgacctgggagagaggggaggtgggggatggttgggaggaatgaaaggaagagagatggcagttgggatgtattgtataagagaagaatcagaaagaaaaaaagaaaagctacctgcacccttcaagtgttcctctgtgtgggaggctgtctcagggactacatgggcaccgagaggcatcagtgagggtaggtacttgatgttgtgtccctgaaaacaaggacaggaaatctgctgcatggcctaagatggcaaaatgtggcacaatcaagtaaggcccaggattctgtctgtggtgcagacctgctgtagaatgagctcccagcattcccacttgctgtgtggagacagcatgttgcagagccatgtgaggatgagggtccaggccaggaggatgtcaacccaccaccatgtagccagtgggctgggggagcttgggcccaccaaggagcttgagcagactgacagtgggttatgtacacaagtgggcgtgtcacacaaccgtgcaacacagagaaaatccctgtgatgacaacttctaaaccaccctgaggcaaaaggagtagacaggggattagagcctagcatattggagtcgagtggccatgcagctcttggaagcgtgaggaaggaaatttcctggaaggataggttgtcttcctagcagcctcgtcaatagatgtcaatgtatgaggtagtacctgctacaatcctgcttcttcagaagactgaggcagggggattacttgaacccagaagttctaggccagtatggacaacatagcaagagtctgatttaaaaaaaaaaaaaaagtaaagagggaaaccaaataggtgacgtgccacactagtgtcttcctgctccaagggtcctgggcacatgagcttgcttagtgccagaaaagtcagaggaggagagggcagacagagaccctcgtctccacctcctttgactgactaatggggctggatataatctgttttacaaaaggacagcttttcagagctgtttctatctaaggttgctctgaatagccatctcgaaatatgccagagaagaatatttagaggcggcctattttggtctcccacaaagatttcacagggaaaatgtatttgtgttctatttatacaactaaaaatatgcatcagcccggggaaactggctctttgctgcctttgaagtgaaggatgggttaatttctaagaaagtaaaagcaaatgtagtgcaggcaccacggatgctgccagacaccagcgtttaagtggcttgaatggaagagcacaccccaagtatttgaagtaggtgaggagagagaccaggtctccagagttgggcctgcagtggccagggtaagtccaaggggcagtatgacgcagaacagagtggcaacctctaccagtagtagaattcaggtctcattcctaacctcccataaagcagagaatattgcactctctttctctctctctctctctctctctctctctctctctctctctctctctctctctctaactcacagagatcctcctgcctctgcttcctcattgctggaaataaaagcatgtgccaccacatgcagctctccttgtttggagagagaaaggcagacagacagacagacagacagacagacagagtataatgtgtgcagatgtccacagaactccgaagagggtgtgggacccccagaaaccggagttctaggcagttgtaagctgtccagtgggtgttgcgaactgaactcaagtcctctggaaaactggaaagtactcttaaccactaagctatctgttagtcccccaagaatgtcttatcttgataggccttcagatctcacagtccagggggctgactgctacaaggggtagaggaaagaaaactggccccaggcctgagttcagagtgacatctccagagttcttccttcccttgccgtgtagtaatttcctaccctacacgaggagaaaaggaacagatatgtccagtatgcctggcatcttgaaagggcactgactcttgccgctgtagagcccctttccttggggagtgcagagaagtgctgctagagaggttcaaaagaaacacaacagtcaaaatagttgctggccaagggagggcatgtgccctgtatcgggcctgcaaagccctttcaacagtgtagccaaggccatgtttgacagtacaagcctgtaagcccatgcataagcaagggctgggataaagggctactgttcaagttagttatatacacatcaagtttgttcatcttacatccttaggtaagggtgggtagcttgttagctccacctctccagcaggaaagcatgtccacggtaaggtagatactgtagagtttagctttgttttgacctttctttcttcgttgcttgggattgtacttgggaacttcactcatgcagcccatgtttcagccttgcataaatctataaatatatggttggagtcaggtccagcatatgtgggtaccagtcccagtctggtatagcaatttattagctatttgtctttagacaagtaactatagggttctgagctgtaatggccaccaaacaggtcctttaagtatctttattggccaaacaccagggttaaaacactgaacacagtatggtggtttcaactgtcatggagtccacattctagcaggggaattttacacacaactcacaaatgatacagaccacagcagggtgatttgacagagacaacgaagggcttgcctgagctgcagtagtccgggagcagctcttgatgggatctgagatctagatggcagagcgcccttcctagatgaaatccaagcctctggttactcctcagcaagagtagctggaggaagaccagaagtgaggaggaaaaggctcagagtggagcatctaagcctgcagcacctccagagacaggctttggagcttgcctgtggttcagcctactgtgggaagagaccagtgagggttttaagctgtggggatgggtatattttggaaggcgaacaagaacaccagcagctcaggcaatgtaggagggcgggaaggtgatggatgatcgaggacggccccagagattttaaggactgtgtaggtggggtgagaagcactgtgggtgaggtggagttagagggaagaagtgaagatctatttttttggccatgaggaatttggggtgcacacacacacatacatacacaccccatcatgtccttgtctacagacggggtgataatggtactggaaggtagaaggtggaaagaggaccagcaaggaaggtatccagtgcccaccccacagctcaccctcagacagaccttgttctcattttcattacccaggtgcccggagaggccagtcccatcacc

SEQ ID NO: 6 - DNA, 22 bp, Artificial Sequence
  Description of Artificial Sequence  Synthetic Primer
atctttgcccggggcttgtcct

SEQ ID NO: 7 - DNA, 50 bp, Artificial Sequence
  Description of Artificial Sequence  Synthetic Primer
gtctgggccaggatgacgggccaactatctttgcccgggcttgtccttca

SEQ ID NO: 8 - DNA, 12 bp, Artificial Sequence
  Description of Artificial Sequence  Synthetic Primer
cactagaaggtt

SEQ ID NO: 9 - DNA, 13 bp, Artificial Sequence
  Description of Artificial Sequence  Synthetic Primer
gtgcgtgggccag

SEQ ID NO: 10 - DNA, 10 bp, Artificial Sequence
  Description of Artificial Sequence  Synthetic Primer
tcagggagag

SEQ ID NO: 11 - DNA, 12 bp, Artificial Sequence
  Description of Artificial Sequence  Synthetic Primer
gtgcgcctatct

SEQ ID NO: 12 - DNA, 23 bp, Artificial Sequence
  Description of Artificial Sequence  Synthetic Primer
tactcctcagcaagagtagctgg

SEQ ID NO: 13 - DNA, 23 bp, Artificial Sequence
  Description of Artificial Sequence  Synthetic Primer
gctgaacttgtggccgtttacgt

SEQ ID NO: 14 - DNA, 21 bp, Artificial Sequence
  Description of Artificial Sequence  Synthetic Primer
cttctatagccttcccaagcc

SEQ ID NO: 15 - DNA, 20 bp, Artificial Sequence
  Description of Artificial Sequence  Synthetic Primer
ctcgtaggtctcacaggaag

SEQ ID NO: 16 - DNA, 25 bp, Artificial Sequence
  Description of Artificial Sequence  Synthetic Primer
cctgctggattacattaaagcactg

SEQ ID NO: 17 - DNA, 25 bp, Artificial Sequence
  Description of Artificial Sequence  Synthetic Primer
gtcaagggcatatccaacaacaaac

SEQ ID NO: 18 - DNA, 24 bp, Artificial Sequence
  Description of Artificial Sequence  Synthetic Primer
ggcgttctctttggaaaggtgttc

SEQ ID NO: 19 - DNA, 20 bp, Artificial Sequence
  Description of Artificial Sequence  Synthetic Primer
ctcgaaccacatccttctct

SEQ ID NO: 20 - DNA, 23 bp, Artificial Sequence
  Description of Artificial Sequence  Synthetic Primer
ttgctgcacgagggctcagaatc

SEQ ID NO: 21 - DNA, 23 bp, Artificial Sequence
  Description of Artificial Sequence  Synthetic Primer
tccgattctcatgctctggcttg

SEQ ID NO: 22 - DNA, 23 bp, Artificial Sequence
  Description of Artificial Sequence  Synthetic Primer
cagccctgtctttgccacgtttg

SEQ ID NO: 23 - DNA, 23 bp, Artificial Sequence
  Description of Artificial Sequence  Synthetic Primer
tccactggattcatcccgctctg

SEQ ID NO: 24 - DNA, 23 bp, Artificial Sequence
  Description of Artificial Sequence  Synthetic Primer
cttcctcttgcaacagagaaccc

SEQ ID NO: 25 - DNA, 23 bp, Artificial Sequence
  Description of Artificial Sequence  Synthetic Primer
actcaacgtccactttgagatgc

(19) United States
(12) Patent Application Publication
(10) Pub. No.: US 2007/0287150 A1
(43) Pub. Date: Dec. 13, 2007

(54) Methods And Compositions Involving S-Ship Promoter Regions

(76) Inventor: Larry R. Rohrschneider, Mercer Island, WA (US)

Correspondence Address: FULBRIGHT & JAWORSKI L.L.P., 600 CONGRESS AVE., SUITE 2400, AUSTIN, TX, 78701, US

(21) Appl. No.: 10/593,202

(22) PCT Filed: Mar. 18, 2005
(86) PCT No.: PCT/US05/08977

§ 371 Date: Jul. 25, 2007

Related U.S. Application Data
(60) Provisional application No. 60/554,318, filed on Mar. 18, 2004.

(51) Int. Cl. C12N 15/11; C12N 15/64; C12N 5/10; C12Q 1/68

(52) U.S. Cl. 435/6; 435/243; 435/320.1; 435/325; 536/24.1

(57) ABSTRACT

The present invention concerns s-ship promoter compositions and methods using the promoter. It includes polynucleotides, vectors, host cells, and transgenic animal including an s-ship promoter controlling the expression of a heterologous nucleic acid. Methods of the invention concern methods of expressing a heterologous nucleic acid is a tissue-specific, developmental-specific, or temporally controlled manner. Other methods includes screening methods and therapeutic methods.

The present application claims priority to U.S. Provisional Patent Application 60/554,318, filed on Mar. 18, 2004, which is hereby incorporated by reference in its entirety.

The government may own rights in the invention pursuant to grant number CA82499 from the National Institutes of Health.

### BACKGROUND OF THE INVENTION

1. Field of the Invention

The present invention relates generally to the fields of molecular and developmental biology. More particularly, it concerns methods and compositions involving s-SHIP promoter regions, which can be used to promote transcription in particular cell types and at particular times during development.

2. Description of Related Art

Stem cells have been the focus of tremendous interest in recent years because of the progress made in developmental and molecular biology and the promise of therapeutic applications in a wide variety of contexts, from heart disease to diabetes, and cancer to Parkinson's disease (see generally Abbott et al., 2003; Daley, 2003; Hirai, 2002; Kondo et al., 2003; Nakano, 2003). Toward fulfilling this promise, many researchers have engaged in extensive studies to characterize factors and pathways in stem cell development and to evaluate candidate therapeutic and diagnostic agents. Such agents include proteins that are gene products, sometimes heterologous, in the stem cells. The ability to express a transgene in stem cells is critical for providing data toward these endeavors. The study of genes normally expressed in stem cells has yielded not only information regarding the developmental, cellular, and molecular biology of these cells, but also useful tools for further studies.

Pathways involved in stem cell function include the protein phosphatidylinositol 3-kinase (PI3K), which becomes activated through cell surface receptors. PI3K is involved in the generation of phosphatidylinositol 3, 4, 5-triphosphate, which activates signaling pathways leading to cell proliferation. The SH2-containing inositol 5′-phosphatase (SHIP1) removes the phosphate group from the D5 position of phosphatidylinositols, which is considered an significant feedback mechanism on cell activation for hematopoietic cells (Lioubin et al., 1996; Rohrschneider et al., 2000).

A form of SHIP1 lacking the SH2 domain has been identified and referred to as stem or short SHIP (s-SHIP) (Tu et al, 2001). Tu et al. found the protein contains amino acids encoded by exons 6-27 of SHIP1 and that it is expressed in embryonic and hematopoietic stem cells. It was initially unclear how s-SHIP protein was produced from the ship1 gene. Kavanaugh et al. (1996) suggested that SIP-110 was a spliced product of SHIP1; however, Tu et al (2001) proposed, based on its cDNA sequence, that it was transcribed from a promoter within the SHIP1 gene. This was inferred from the fact that the first 44 nucleotides of the s-SHIP cDNA were at the 5′ end immediately before exon 6 of SHIP1. These 44 nucleotides were not contained in the SHIP1 cDNA, but were identical to the 44 nucleotides of genomic ship1 intron 5, immediately adjacent to exon 6. However, no functional evidence for an s-ship promoter was provided. Therefore, while a promoter with the tissue-specific expression of s-ship could be valuable from both a research and therapeutic/diagnostic perspective, further investigation of the s-ship gene was required to identify the promoter and characterize any tissue-specific activity.

Moreover, until now, a promoter providing the developmental-specific expression of the s-ship promoter, which includes expression in stem cells, has not previously been described. Thus, the present invention addresses these issues.

### SUMMARY OF THE INVENTION

The present invention concerns methods and compositions involving a functional and isolated s-ship promoter. The invention includes nucleic acid molecules, host cells, and transgenic organisms having an s-ship promoter, as well as methods of using the promoter for transcription, expression studies, stem cell analyses, and therapeutic applications. In addition the present invention relates to methods and compositions involving an isolated and function promoter capable of directing transcription in a) adult and embryonal stem/progenitor cells and in b) in adult and embryonal stem/progenitor cells that have differentiated to the point where the promoter directs transcription only when a developmental decision is required, i.e., when the cell is in the resting or growing phase, or the transition from the resting to the growing phase. This promoter will be referred to as a “developmental decision promoter.” S-ship promoter regions discussed herein constitute a developmental decision promoter. Thus, it is contemplated that embodiments discussed with respect to an s-ship promoter can be applied more generally to a developmental decision promoter. Consequently, the present invention covers those embodiments with respect to development decision promoters.

The present invention concerns an s-ship promoter and its functional derivatives. The term “promoter” is used according to its ordinary and plain meaning to a person skilled in the art of eukaryotic transcriptional regulation. The terms “s-ship promoter” or “s-ship1 promoter” refer to the nucleic acid sequence from the s-ship gene that is capable of promoting transcription of a nucleic acid sequence that is connected to it (downstream). Transcription can be assayed according to any number of ways known to those of skill in the art, including, but not limited to, an expression assay using a screenable or selectable marker; ribonuclease protection assay (RNAP), RT-PCR, and in vitro transcription reactions, all of which are well known to those of skill in the art and can be implemented using commercially available reagents and protocols (see generally, Sambrook et al., 1989; Ausubel, 1992 and 1994, all of which are incorporated by reference).

Compositions of the invention include isolated polynucleotides comprising an s-ship promoter capable of promoting transcription. SEQ ID NO:1 is a 102 kb genomic mouse ship1 sequence. In certain embodiments, the s-ship1 promoter comprises, or has at least or at most 5, 6, 7, 8, 9, 10, 11, 12, 13, 14, 15, 16, 17, 18, 19, 20, 21, 22, 23, 24, 25, 30, 40, 50, 60, 70, 80, 90, 100, 110, 12, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, or more contiguous nucleotides of the ship1 gene, including SEQ ID NO:1-23, or any range derivable therein. In specific embodiments, s-ship1 promoter includes one or more of the following regions of SEQ ID NO:1: from nucleotide (nt) 49485 to 61006 (11.5 kb-GFP construct); 49485 to 57111, which is 7626 nt (7.6 kb construct); from nt 49485 to 55810, which is 6326 nt (6.3 kb construct); from 54807 to 61006, but lacking 57109 to 57944 (6.2 kb-GFP construct); from nt 51389 to 55810, which is 4421 nt (4.4 kb construct); from nt 52199 to 56423, which is 4224 nt (4.2 kb construct); from nt 53820 to 55810, which is 1990 nt (1.9 kb construct); from nt 54755 to 55810, which is 1055 nt (0.96 kb construct); or from nt 55668 to 55810, which is 142 nt (44 nt construct). It is further contemplated that the lengths of contiguous nucleotides discussed above can be applied with respect to these identified regions of SEQ ID NO:1, as well as any other sequence disclosed herein.

Moreover, any of these lengths or regions discussed in the context of SEQ ID NO:1, apply to the corresponding regions of SEQ ID NO:2, SEQ ID NO:3, and SEQ ID NO:4. SEQ ID NO:2 includes the genomic sequence for the mouse ship gene from exon 5 through exon 7 (exon 5, intron 5, exon 6, intron 6, and exon 7 inclusive). SEQ ID NO:3 is a mouse s-ship promoter sequence that includes the 560 nucleotides upstream of exon 6 (in intron 5). SEQ ID NO:4 is a human s-ship promoter sequence that includes the 560 nucleotides upstream of exon 6 (in intron 5). SEQ ID NO:5 is the mouse s-ship promoter region in the 11.5 kb-GFP construct. While these s-ship promoters provided are from human and mouse, the invention, is not limited to these species. It is contemplated that mammalian s-ship promoters are contemplated, particularly those with homology to the sequences disclosed in the application. SEQ ID NO:6 is the sequence from intro 5 of s-ship that has a p53 family binding motif 5′-ATCTTTGCCC/GGGGCTTGTCCT-3′, meaning that members of the p53 family of proteins have been shown to bind to sequences homologous or identical to this sequence. SEQ ID NO:7 is a sequence from the s-ship promoter that is homologous to a Pax8 binding sequence (5′-CACT/AGAAGGTT-3′). SEQ ID NO:8 is a sequence from the s-ship promoter that is homologous to a Smad 3/4 binding sequence (5′-GT/GC/GTGGGCCAG-3′). SEQ ID NO:9 is a sequence from the s-ship promoter that is homologous to a Stat 1/5 binding sequence (5′-TCAGGGA/GAG-3′). SEQ ID NO:10 is a sequence from the s-ship promoter that is homologous to a GATA/Lmo2 sequence (5′-GTGC/GCCTATCT-3′). It is understood by those of skill in the art that the convention of using a slash (/) indicates two alternative nucleotides at that position, where the slash separates the alternatives. It is also contemplated that sequences of the invention may include one or more of these consensus sequences for these binding sites. In certain embodiments, these motifs may be repeated singly or in combination with one another.

Moreover, it is contemplated that the promoter contains enhancer activity. In some embodiments of the invention, 10, 20, 30, 40, 50, 60, 70, 80, 90, 100, 110, 12, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600 contiguous nucleotides, or any range derivable therein, from the region including the first 600 nucleotides upstream of exon 6 are included in s-ship promoters of the invention. It is also contemplated that segments that are at least 80%, 85%, 90%, 95%, 96%, 97%, 98%, 99% identical to any of these regions or capable of hybridizing to the complement of such a regions are contemplated as part of the invention. Such segments may further comprise sequences in intron 6, exon, 5, exon 6 or other regions of the ship1 gene.

It is contemplated that functional derivatives of the s-ship promoter also contemplated by the invention. Functional derivatives of an s-ship promoter may be at least 80%, 85%, 90%, 95%, 96%, 97%, 98%, 99% identical to the polynucleotides discussed herein. Such derivatives may also be characterized by any of the lengths of contiguous nucleotides discussed above. Moreover, polynucleotides of the invention include those that are capable of hybridizing to all or part of the recited lengths of SEQ ID NOs:1-10 discussed herein, including those particular regions recited in the previous paragraph. Such polynucleotides may be capable of hybridization under high, medium or low stringency conditions.

In specific embodiments, the s-ship promoter is capable of promoting tissue-specific transcription. Transcription may be accomplished, in some embodiments of the invention, in skin, a hair follicle, cornea, embryo, gonads, mammary gland, pancreas, and/or vascular smooth muscle. It is also contemplated that transcription may be achieved in cells qualified as or with characteristics of stem cells, which may or may not be derived from skin, a hair follicle, cornea, embryo, gonads, mammary gland, pancreas, and/or smooth muscle. In some embodiments, transcription is achieved in a hematopoietic cell in a tissue-specific manner, including in hematopoietic stem or progenitor cells, but also in more mature or differentiated cells.

It is specifically contemplated that the s-ship promoter directs transcription in a developmentally and/or cell-cycle-dependent manner. In some embodiments, the s-ship promoter directs transcription in stem or progenitor cell that differentiates to a point so that the promoter no longer provides expression at all or provides expression during certain times, such as when it is preparing for a growth and/or developmental phase. As discussed above, the invention concerns developmental decision promoters such as s-ship and embodiments discussed with respect to s-ship can be applied respect to developmental decision promoters, and vice versa.

In some embodiments, the present invention includes a promoter that is capable of directing transcription in cells that qualify as stem or progenitor cells and/or cells that have undergone some differentiation but are not terminally differentiated and that are not in a resting state. In some embodiments, the invention provides isolated poly nucleotides, expression cassettes, vectors and host cells comprising a heterologous nucleic acid sequence under the control of a developmental decision promoter.

In these embodiments, the developmental decision promoter is capable of providing expression in embryonic stem cells. In other embodiments, the promoter is capable of providing expression in adult stem cells. It is contemplated that the adult stem cells are differentiated but not terminally differentiated; in other words, they are self-renewing and capable of being differentiated into other cell types derivative of the stem cell. For example, the adult stem cell may be a hematopoietic or epidermal stem cell meaning it is capable of self-renewing and becoming any hematopoietic or epidermal skin cell. The term “terminally differentiated” is used according to its ordinary and plain meaning according to those of ordinary skill in the art.

In other embodiments, the developmental decision promoter is capable of providing expression in adult stem cells that are in growing phase (i.e., in a non-resting phase of mitosis or meiosis). In certain other cases, the promoter is capable of providing expression in a cell from mouse embryonic development stages E3-E18.5. The E numbers refer to age of the embryo, based on days, from approximate conception, for example, as set forth on the World Wide Web at the following address: genex.hgu.mrc.ac.uk/CDROM_online/macd/html_shdw_links/mastaging.html. It is contemplated the expression may be achieved at mouse embryonic development stage E1, E2, E3, E3.5, E4, E4.5, E5, E5.5, E6, E6.5, E7, E7.5, E8, E8.5, E9, E9.5, E10, E10.5, E11, E11.5, E12, E12.5, E13, E13.5, E14, E14.5, E15, E15.5, E16, E16.5, E17, E17.5, E18, E18.5, E19, E19.5, E20 or later, or any combination derivable therein, or any corresponding stage of development in another species of mammal. In some embodiments, the promoter can provide expression throughout all these stages of development (constitutive) or through a subset (not constitutive throughout). In particular embodiments, the promoter is capable of directing transcription in stem/progenitor cells of an embryo and also in adult stem cells periodically (non-constitutively) or constitutively.

It is further contemplated that the developmental decision promoter is further capable of providing expression in a cell that is in a developed animal. In other words, a developed animal refers to an animal that has already been born and is no longer an fetus or embryo. Thus, the cell may be in an animal that has already been born and it may be near or surrounded by differentiated cells or tissue. For example, the cell may be a stem or progenitor cell in the developed animal.

In particular embodiments, the developmental decision promoter is an s-ship promoter region comprises a sequence that can hybridize under stringent conditions to nucleic acid segment comprising the complement of i) at least 20 contiguous nucleic acids of SEQ ID NO:1, SEQ ID NO:2, or SEQ ID NO:3; or ii) SEQ ID NO:5, SEQ ID NO:6, SEQ ID NO:7, SEQ ID NO:8, SEQ ID NO:9 and/or SEQ ID NO:10.

In some embodiments of the invention, an s-ship promoter is operably attached to a heterologous nucleic acid. The term “heterologous” is used according to its plain and ordinary meaning to a person skilled in the art of molecular biology. It is a relative term and in the context of an s-ship promoter, it refers to a nucleic acid sequence that is not normally found in nature (with respect to sequence and position) with the s-ship promoter. In other words, it refers to any nucleic acid that is not the entire genomic sequence of the s-ship gene. In some embodiments, the s-ship promoter is connected to a nucleic acid sequence encoding part of the s-ship gene product or all or part of an s-ship1 cDNA sequence. Alternatively, the s-ship promoter may be placed at a location different than is found in nature.

Because recombinant cells and transgenic animals, including knockout versions thereof, are part of the invention, the present invention further encompasses nucleic acids containing an s-ship gene or a portion thereof and a marker sequence, wherein the s-ship gene is disrupted by the marker sequence. In some embodiments, the nucleic acid is under the control of a promoter, which is an s-ship promoter in further embodiments. The promoter may also be constitutive, inducible, or conditional. Promoters discussed herein may be tissue-specific (spatially restricted), developmental-specific (providing transcription at specific developmental stages or times), and/or temporally restricted.

The present invention further concerns expression cassettes, vectors, and host cells that contain or include polynucleotides having an s-ship promoter that has been isolated away from its chromosomal context. The polynucleotides and embodiments discussed above may be implemented with respect to expression cassettes, vectors, and host cells.

It is contemplated that the s-ship promoter may control the transcription of a nucleic acid sequence encoding a marker. In some embodiments, the marker is colorimetric, enzymatic, or fluorescent. Examples include, but are not limited to, β-galactosidase, chloramphenicol acetylase, luciferase, and green fluorescent protein. In further embodiments, a heterologous nucleic acid segment encodes a therapeutic or diagnostic gene product. The therapeutic or diagnostic gene product may be a protein or RNA molecule, such as an siRNA or miRNA molecule. In some embodiments, the therapeutic gene product is selected from the group consisting of a tumor suppressor, an oncogene, a cytokine, a cytokine receptor, a differentiation-inducer, growth factor, and a growth factor receptor. It is contemplated that more than one heterologous sequence or gene may be placed under the control of a promoter. Examples of such proteins are well known to those of skill in the art, and include, but are not limited to, interleukins (IL-2, -6, -8, -9, -10, -11, -12, -13, -14, -15, -16, -17, -18, -19, -20, -21, -22, -23, -24, etc.), interferons, receptor tyrosine kinases and their ligands (kit/steel, CSFR/CSF, GM-CSFR/GM-CSF, PDGFR/PDGF, flk-1/VEGF, Lif, EGF, FGF, etc.), transforming growth factors α and β, Epo, IGF, tumor necrosis factor α and β. A number of examples can be seen on the world wide web at indstate.edu/thcme/mwking/growth-factors.html, which is specifically incorporated by reference. In specific embodiments, it is contemplated that the heterologous encoded protein can transform or immortalize a cell, such as an oncogene. In certain embodiments, a stem cell can be immortalized or transformed.

In some embodiments of the invention, a vector is a plasmid, YAC, BAC, or virus. Viruses include adenovirus, adeno-associated virus, retrovirus, flavivirus, and vaccinia virus.

Compositions of the invention may be prepared in a pharmaceutically or pharmacologically acceptable formulation. Such formulations are well known to those of skill in the art for use in in vivo contexts.

Other aspects of the invention include host cell having an s-ship promoter operably attached to a heterologous nucleic acid segment. In some embodiments, the host cell is eukaryotic, though it may be prokaryotic. In specific embodiments, the host cell is from a mammal, insect, bacteria, or yeast. Cells from monkeys, mice, rats, rabbits, hamsters, ferrets, and humans are specifically contemplated for use with nucleic acids of the invention. In some cases, the host cell is an embryonic cell, which may specifically be a blastocyst cell. In other cases, the host cell is a stem or progenitor cell. In some cases, the cell is a hematopoietic cell, meaning any cell in that lineage. It is contemplated that the cell may be in vitro or in vivo.

Cells that can be used according to methods and compositions of the invention include, but are not limited to, CD34+ cells (cells expressing CD34 on their surface), undifferentiated cells, stem cells, progenitor cells, cord blood cells, placental cells, neonatal or fetal cells, immature cells, pluripotent cells, and totipotent cells. The term “stem cell” is used according to its ordinary meaning, for example, as described by the National Institutes of Health (on the World Wide Web at stemcells.nih.gov). Stem cells 1) are “capable of dividing and renewing themselves for long periods”; 2) are unspecialized; and, 3) can give rise to specialized cell types.

The invention specifically contemplates the use of embryonic stem cells, adult stem cells, or neonatal and fetal stem cells. An adult stem cell typically refers to a stem cell from a particular organ or tissue that is capable of differentiating into one or more cells of that organ or tissue. Umbilical cord blood contains stem cells that are similar to embryonic stem cells in that they are believed to be capable of being differentiated into a number of different cell types, as opposed to cell types of one particular organ or tissue. Umbilical cord blood refers to blood that remains in the umbilical cord and placenta following birth and after the cord is cut. “Placental blood” is understood to be synonymous with cord blood; similarly, cord blood stem cell is considered synonymous with placental or placental blood stem cell. The use of stem cells from umbilical cord blood is specifically contemplated in certain embodiments of the invention. In some but not all cases, the use of other stem cells is specifically not considered part of the invention, particularly the use of pancreatic/endocrine progenitor or stem cells is not considered for use with some embodiments. Furthermore, cells of the invention may be characterized by cell surface antigens. Cell surface antigens and their correlation with cell type and cell development are known to those of ordinary skill in the art.

It will be understood that cultures or samples containing cells discussed above are also contemplated for use according to methods and compositions of the invention.

Further embodiments of the invention include cells for use in the generation of transgenic organisms (knock-in and knock-out). Accordingly, there are recombinant host cells in which one or both s-ship genes is disrupted by marker sequence or in which all or part of an s-ship gene is flanked by an excisable sequence, such as a loxP sequence. The marker sequence serves the purpose of showing when the transgenic sequence is present or absent in the cell.

The present invention further concerns transgenic animals comprising an s-ship promoter operably attached to a heterologous nucleic acid segment. Mammals are specifically contemplated, particularly mice. In some cases, the invention involves a mammal having cells comprising an s-ship transgenic sequence. The s-ship sequence may be knocked in or out in a restricted or controlled manner. For example, whether it is knocked in or out may be controlled in a tissue-specific, inducible, conditional, developmental or temporal manner. Consequently, animals may have heterologous genes under the control of a promoter or system that operates in that way. The Cre-Lox system is one example. The transgene of interest itself may not be under the control of a limited promoter, but a secondary gene whose product initiates the knock-in or knock-out process may be under such a promoter. In one embodiments, animals of the invention may have an s-ship transgenic sequence that includes an s-ship coding sequence flanked by loxP sequences. They may also have a heterologous nucleic acid sequence encoding a Cre recombinase. In some cases, the nucleic acid sequence encoding the Cre recombinase is under the control of an inducible or conditional promoter. Transgenic animals of the invention are not limited by the Cre-Lox system, which serves as an example of how expression may be controlled.

A number of methods are included as part of the present invention. In some embodiments, there are methods for expressing a recombinant nucleic acid in a cell comprising: a) transfecting the cell with an expression cassette comprising an s-ship promoter operably attached to the recombinant nucleic acid, wherein the nucleic acid is transcribed. The cell may be any of the host cells discussed above. Moreover, it is contemplated that embodiments may be carried out with a developmental decision promoter, which may be an s-ship promoter region. In some embodiments there are methods for expressing a nucleic acid in a stem cell comprising providing to a cell a polynucleotide including the nucleic acid under the control of a developmental decision promoter, wherein the nucleic acid is expressed in the cell. It is contemplated that a cell may be in a subject. The cell may have been provided with a nucleic acid in vivo or in vitro. In the latter case, a cell may be introduced into a subject thereafter.

Alternatively, an isolated nucleic acid encoding a developmental decision promoter can be provided to cell such that the promoter integrates into the cell's genome to drive expression of a gene that becomes operably linked to the promoter. The present invention covers methods and compositions for implementing the expression strategy.

Other embodiments of the invention concern methods of screening for a candidate substance that regulates activity of the s-ship promoter comprising a step selected from the group consisting of: (a) contacting a nucleic acid comprising an s-ship promoter with an s-ship promoter binding protein and the candidate substance under conditions that allow binding between the protein and the promoter and determining whether the candidate compound modulates the binding between the protein and the promoter; and (b) contacting the candidate substance with a cell comprising the s-ship promoter operably attached to a reporter gene coding for an expression product and assaying for expression of the reporter gene expression product. One or both steps may be employed. Ways of determining whether the candidate compound modulates binding between a protein and the promoter are well known to those of skill in the art. The compound may inhibit, reduce, decrease, eliminate, increase, promote, tighten the binding between the protein and the promoter. Assays for such an interaction include, but are not limited to, electrophoretic mobility shift assays (EMSA), DNA footprinting, functional transcription assays—as described above—Southwestern assays, and PCR-based assays.

The present invention also includes methods for identifying stem cells in a population of cells comprising: (a) administering to cells in the population a nucleic acid comprising an s-ship promoter operably attached to a reporter or marker gene. The reporter or marker gene is then used to identify positively-expressing cells, which would indicate the cell is a stem cell. The cell may be in an organ and/or in an animal. In some embodiments, methods include sorting cells based on expression of the reporter or marker gene. In addition to the assays discussed above, FACS analysis may be employed, in addition to other cell sorting techniques. Methods include differentiation of the cells.

Aspects of the invention also concern methods for screening for a modulator of cell function comprising: a) transfecting a stem or hematopoietic cell with an expression cassette comprising an s-ship promoter operably attached to a nucleic acid encoding a candidate modulator; and, b) assaying the cell for a cell function, wherein a difference in cell function in the cell as compared to a cell in the absence of the candidate modulator is indicative of a modulator. The term “modulator” refers to a substance that affects cell function. It may affect cell function by acting on or through a pathway. The modulator may inhibit, reduce, eliminate, decrease, increase, promote, induce, or enhance a particular cell function or result of a pathway in the cell. It is contemplated that this method may be employed to identify a modulator as a candidate therapeutic agent for the treatment of a blood-related disease or condition.

Therapeutic methods are also provided by the present invention. Methods are not necessarily limited to a particular disease or condition. It is contemplated that any method in which expression in stem cells or cells in which the s-ship promoter can function are contemplated for use in therapeutic methods of the invention. For example, the method may be applied to pancreatic disorders and diseases.

Thus, in some embodiments, there is a method of treating a patient with a blood-related disease or condition comprising: a) transfecting a cell with an expression cassette comprising an s-ship promoter region operably attached to a therapeutic nucleic acid; and, b) administering the cell to the patient. Blood-related disease or condition include blood-related cancers—such as leukemia, lymphoma, or myeloma—and anemia. In some cases, the blood-related condition can be treated using stem cell replacement therapy.

Other methods of the invention include providing a method of treating a tumor comprising providing to stem cells of the tumor an agent that promotes their destruction. In some embodiments a patient with a tumor is provided with a host cell or expression construct comprising a developmental decision promoter such as an s-ship promoter region that provides expression for a therapeutic agent in the tumor stem cell. The therapeutic agent may a protein or a nucleic acid. In certain embodiments, the agent promotes apoptosis or cell death of the tumor stem cell, such as with a toxin or apoptosis inducer.

Other methods of the invention include ways of tracking stem cells or isolating stem cells. Expression using developmental decision promoters can be used to track or isolate stem cells by virtue of their expressing a product that can be tracked or used to isolate the stem cells. In terms of tracking, the product may be some kind of reporter, which may or may not be a cell surface marker. In the case of isolating stem cells, the product will allow the stem cells to be separated from non-stem cells, such as by FACS analysis based on an expressed cell surface marker.

Cells for therapeutic use may, in addition to the cells discussed above, be bone marrow cells, or be autologous or allogeneic.

It is contemplated that any embodiment discussed with respect to any method or composition described herein can be implemented with respect to any other method or composition described herein. For example, an embodiments discussed with respect to an s-ship promoter region applies to a developmental decision promoter, and vice versa. Similarly, an embodiments discussed with a polynucleotide, primer, expression constructs, host cells, transgenic organisms, and method of the invention are contemplated for use with any other polynucleotides, primers, expression constructs, host cells, transgenic organisms, and methods of the invention, and vice versa.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law convention, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. ship1 genomic segments cloned into a promoter-less expression vector for testing cell-specific promoter activity. The upper line represents the general ship1 genomic region containing potential activity for cell-specific s-SHIP expression. Intron 5 contains the likely promoter activity and transcription is proposed to begin before exon 6. The 44-intronic nucleotides, contained in the s-SHIP cDNA, are shown as red. A 7.6 kb genomic fragment (second line down), as well as the indicated sub-fragments, were cloned into a promoter-less plasmid for GFP expression. The design and construction of the plasmid is detailed in Materials and Methods.

FIG. 2. Flow cytometry analysis of cell type-specific promoter activity in D3 ES cells vs. NIH13T3 cells. Each construct shown was cloned into a promoter-less GFP plasmid, which was linearized and electroporated into D3 ES cells, or transfected into NIH3T3 cells. G418 resistant colonies were then examined by flow cytometry for GFP expression. Two different “empty vector” negative controls were utilized depending on whether the insert contained a splice acceptor or both splice acceptor and donor. Both these plasmids without genomic insert were negative for GFP expression in both cell types, but only a single negative-control vector is shown. Two positive-control plasmids were utilized in each experiment. These controls expressed GFP from an IRES, and one expressed a protein insert, both were positive in each cell type.

FIG. 3A-B. Structure of the 11.5-kb and 6.2-kb transgenic promoter-GFP constructs for in vivo analysis. FIG. 3A. Two promoter transgenic constructs were prepared. The first construct is called the 11.5 kb-GFP transgene, and contains the entire genomic ship1 segment from the Sac I site near the 5′ end of intron 5 through the putative translation start site at an ATG preceded by a suitable Kozac sequence within exon 7. The translational start ATG for the enhanced GFP is fused, in frame, to the likely ATG translational start for s-SHIP. A second transgenic construct, called the 6.2 kb-GFP transgene, is identical to the 11.5 kb-GPP construct, except it contains only 0.96 kb upstream of exon 6, and lacks 833 nt within intron 6. FIG. 3B. Transgenic copy numbers were estimated by semi-quantitative PCR analysis relative to the endogenous diploid gab2 gene.

FIG. 4. Computer analysis of 600 nucleotides of the intron-5 transgene promoter region. A. The region immediately upstream of exon 6 is shown with potential transcription factor binding motifs determined by analysis using the MatInspector program. Only the factors with matrix and core similarity greater than 0.9 are shown. Those factor motifs within the strand shown are over-lined, while those factors potentially interacting with the complementary strand are shown underlined. The SSR or stem-SHIP region identified by Tu et al., 2001 is in bold, and an initiator sequence for transcription is situated at the beginning of the SSR. Exon 6 (not shown) begins at the 3′ end of the SSR.

FIG. 5. The two primary proteins, s-SHIP and SHIP1, are produced from the ship1 gene. The domain structure of the two proteins is shown above the ship1 genomic intron/exon organization. Transcription for the full-length 145 kDa SHIP1 protein initiates in promoter 1 (Prol), utilizes all 27 exons, and translation begins in the exon 1 encoded region. The stop codon is the first three nucleotides of exon 27. Transcription for the s-SHIP protein begins within intron 5 (Pro 2), and downstream is identical to the SHIP1 product. Translation, however, presumably begins in the first ATG of exon 7. Both transcripts and protein sequences are therefore identical from the ATG in exon 7 through the stop codon in exon 27. The dashed lines indicate translation start and stop points for each protein within the genomic exons.

FIG. 6. The 560-nucleotide regions immediately upstream of exon 6 from the mouse and human sequences were compared. Inr indicates the initiator sequence. Binding sites are also identified.

FIG. 7. p53 binding sequences with half sites are depicted. This sequence is SEQ ID NO:7.

### DESCRIPTION OF ILLUSTRATIVE EMBODIMENTS

The present invention is based on the isolation and characterization of the s-ship1 promoter, which can be used to promote transcription. Methods and compositions involving the s-ship1 promoter are provided herein. In some embodiments, they take advantage of the tissue specificity of s-ship1 expression. s-ship1 encodes a protein whose expression has been observed in limited cell populations, and thus, the tissue-specificity of its promoter can be exploited in a number of different ways.

### I. SHIP1 AND s-SHIP BACKGROUND

The s-ship1 promoter was studied because of the function and expression patterns for the s-ship1 (also referred to as s-ship) and ship1 gene products. The murine SHIP1 protein is encoded in 27 exons of the Inpp5d (inositol polyphosphate-5-phosphatase D) locus, spanning approximately 102 kbps on chromosome 1 at position 57.0 cM of the genetic map, or cytoband C5 of the cytogenetic map (reviewed in Rohrschneider et al., 2000; Wolf et al., 2001; NCBI databases). The full-length protein is 145 kDa, but splicing, involving exons 25 and 26, can produce 4 additional proteins ranging in size from 109-135 kDa (Lucas and Rohrschneider, 1999; Wolf et al., 2000). These splicing reactions affect the 350-amino acid C-terminal tail region and its numerous protein interaction motifs, such as those binding PTB, SH2, and SH3 domains.

The prominent structural features of the SHIP1 protein dictate its major functional interactions. The SHIP1 SH2 domain has general specificity for tyrosine-phosphorylated Yxx(L/I/V) amino acid motifs, and its inositol 5′ phosphatase domain removes phosphate from the 5′ position of inositol(3,4,5)P₃, phosphatidylinositol(3,4,5)P₃ or Inositol(1,3,4,5)P₄ [see Sly et al., (2003) for review]. The tyrosine-phosphorylated C-terminal tail interacts directly with the PTB domain of Shc and Dok proteins (Lioubin et al., 1996; Sattler et al., 2001; Tamir et al., 2000), and a potential interaction motif for the SH2 domain of the p85 component of the p85/PI3K is present in the full-length SHIP1 (Gupta et al., 1999; Lucas and Rohrschneider 1999), but eliminated by the splicing events producing the α and β isoforms (Rohrschneider et al., 2000). Polyproline-rich interaction motifs for the SH3 domains of Grb2 also are present in the C-tail region (Kavanaugh et al., 1996). The SHIP1 proteins (e.g., the 145 kDa protein and isoforms thereof) are expressed in hematopoietic cells and testes, with lower expression observed in a few other adult tissues (Q. Liu et al., 1998, reviewed in Rohrschneider, 2003).

Functionally, both biochemical and genetic experiments indicate SHIP1 is a negative regulator of myeloid cell proliferation, survival, and perhaps chemotaxis (see Sly et al., 2003; Rohrschneider, 2003). Also, SHIP1 negatively regulates degranulation, inflammatory cytokine release, and adhesion for mast cells, and SHIP1 is a component of negative signaling (anergy) in B cell proliferation. The molecular mechanisms for most of these effects require the attachment of the SHIP1 SH2 domain to the cytoplasmic portions of transmembrane receptors containing appropriate tyrosine-phosphorylated interaction motifs. There, the SHIP1 inositol-5′phosphatase domain converts the plasma membrane PI3K-produced substrate, phosphatidylinositol(3,4,5)P₃ to phosphatidylinositol(3,4)P₂ effectively terminating proliferation signals. Therefore, the SH2 domain of SHIP1 plays a critical role in initiating many of these negative biological effects.

An additional smaller protein from the ship1 locus is described as an SH2-less 104 kDa protein (Tu et al., 2001). This product is called s-SHIP, with the prefix signifying its only known expression within two stem cell types (i.e., ES cells and lineage-depleted Sca1-positive cells of the bone marrow). This protein was first described by Kavanaugh et al. (1996) and called SIP-110 in the human; but details of its existence were unclear until Tu et al. (2001) defined the cDNA and demonstrated endogenous expression in the two cell types described above. Thus, structurally, s-SHIP differs from SHIP1 only by the lack of the N-terminal SH2 domain; but biochemically, s-SHIP also lacks tyrosine phosphorylation and association with Shc (Kavanaugh et al., 1996; Tu et al., 2001). Nevertheless, s-SHIP constitutively interacts with Grb2. The lack of an SH2 domain in s-SHIP indicates its interaction mechanism with target proteins probably differs from that of SHIP 1; however, the biological functions of s-SHIP are not known.

### II. NUCLEIC ACIDS

A. Polynucleotides

The s-ship1 promoter was identified as a strong promoter for s-SHIP by analyses of the genomic ship1 intron-5 region in driving GFP expression both in vitro and in vivo. This promoter exhibited cell-type specific expression in ES cells, and mice transgenic for the promoter (the 11.5 kb-GFP transgene) showed tissue-specific GFP expression within the inner cell mass of the blastocyst. Transgenic mice produced with a shorter promoter construct (the 6.2 kb-GFP transgene) expressed GFP throughout the blastocyst, suggesting the absence of negative regulatory regions in the shorted transgene. RT-PCR analysis demonstrated s-SHIP expression within the blastocyst. These results indicate that the 11.5-kb promoter region of the transgene contains the information for tissue-specific expression of s-SHIP, as well as tissue-specific shut-off of this protein. It is specifically contemplated that this promoter and the transgenic mice will be useful for future examination of GFP-expression in potential stem/progenitor cells of the embryo and the adult mouse.

The present invention concerns polynucleotides, isolatable from cells, that are free from total genomic DNA and that contain an s-ship promoter. It is contemplated that the s-ship1 promoter is capable of directing transcription of nucleic acid sequence. Transcription may be directed in a tissue-specific or developmental manner. The nucleic acid sequence may encode a peptide or polypeptide, or it may also encode an RNA molecule that is not translated into a protein.

A “promoter” is a control sequence that is a region of a nucleic acid sequence at which initiation and rate of transcription are controlled. It may contain genetic elements at which regulatory proteins and molecules may bind, such as RNA polymerase and transcription factors. The phrases “operatively positioned,” “operatively linked,” “under control,” “operatively attached,” and “under transcriptional control” mean that a promoter is in a correct functional location and/or orientation in relation to a nucleic acid sequence to control transcriptional initiation and/or expression of that sequence. Typically, the promoter is located 5′ or upstream from the strand of sequence to be transcribed. A promoter may or may not be used in conjunction with an “enhancer,” which refers to a cis-acting regulatory nucleic acid sequence involved in the transcriptional activation of a nucleic acid sequence. More particularly, it refers to a nucleic acid sequence that is tissue-specific and stimulates transcription regardless of orientation (forward or reverse orientations both work). The inventors believe that within the first 600 nucleotides upstream of exon 6 there is enhancer activity. Consequently, it is contemplated that all or part of this region may be included in nucleic acid construct containing segment of SEQ ID NO:1.

As used herein, the term “DNA segment” or “nucleic acid segment” refers to a DNA or nucleic acid molecule that has been isolated free of total genomic DNA of a particular species. Therefore, a DNA segment encoding a polypeptide refers to a segment that contains wild-type, polymorphic, or mutant polypeptide-coding sequences yet is isolated away from, or purified free from, total mammalian or human genomic DNA. Included within the term “DNA segment” are a polypeptide or polypeptides, DNA segments smaller than a polypeptide, and recombinant vectors, including, for example, plasmids, cosmids, phage, viruses, and the like.

As used in this application, the term “s-ship polynucleotide” refers to an s-ship-encoding nucleic acid molecule. The term “cDNA” is intended to refer to DNA prepared using messenger RNA (mRNA) as template.

It also is contemplated that a particular polypeptide from a given species may be represented by natural variants that have slightly different nucleic acid sequences but, nonetheless, encode the same protein.

Similarly, a polynucleotide comprising an isolated or purified wild-type, polymorphic, or mutant polypeptide gene refers to a DNA segment including wild-type, polymorphic, or mutant polypeptide coding sequences isolated substantially away from other naturally occurring genes or protein encoding sequences. In this respect, the term “gene” is used for simplicity to refer to a functional protein, polypeptide, or peptide-encoding unit. As will be understood by those in the art, this functional term includes genomic sequences, cDNA sequences, and smaller engineered gene segments that express, or may be adapted to express, proteins, polypeptides, domains, peptides, fusion proteins, and mutants. A nucleic acid encoding all or part of a native or modified polypeptide may contain a contiguous nucleic acid sequence encoding all or a portion of such a polypeptide of the following lengths: about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs.

In particular embodiments, the invention concerns isolated DNA segments and recombinant vectors incorporating an s-ship promoter with a heterologous nucleic acid sequence or a ship or s-ship cDNA segment. Thus, an isolated DNA segment or vector containing a DNA segment may encode, for example, the heterologous nucleic acid sequence. The term “recombinant” may be used in conjunction with a polypeptide, the name of a specific polypeptide, a nucleic acid sequence, or a host cell, and this generally means that the entity involves or involved a nucleic acid molecule that was manipulated in vitro using recombinant DNA technology.

The nucleic acid segments used in the present invention, regardless of the length of the coding sequence itself, may be combined with other nucleic acid sequences, such as enhancers, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

It is contemplated that the nucleic acid constructs of the present invention may encode full-length polypeptide from any source or encode a truncated version of the polypeptide, such that the transcript of the coding region represents the truncated version. The truncated transcript may then be translated into a truncated protein. Alternatively, a nucleic acid sequence may encode a full-length polypeptide sequence with additional heterologous coding sequences, for example to allow for purification of the polypeptide, transport, secretion, post-translational modification, or for therapeutic benefits such as targetting or efficacy. As discussed above, a tag or other heterologous polypeptide may be added to the modified polypeptide-encoding sequence, wherein “heterologous” refers to a polypeptide that is not the same as the modified polypeptide.

In a non-limiting example, one or more nucleic acid constructs may be prepared that include a contiguous stretch of nucleotides of sequences disclosed herein, including the s-ship promoter.

A nucleic acid construct may be at least 20, 30, 40, 50, 60, 70, 80, 90, 100, 110, 120, 130, 140, 150, 160, 170, 180, 190, 200, 250, 300, 400, 500, 600, 700, 800, 900, 1,000, 2,000, 3,000, 4,000, 5,000, 6,000, 7,000, 8,000, 9,000, 10,000, 15,000, 20,000, 30,000, 50,000, 100,000, 250,000, 500,000, 750,000, to at least 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges,” as used herein, means any length or range including or between the quoted values (i.e., all integers including and between such values).

It is specifically contemplated that nucleic acids of the invention may include, be at most, or be at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000 or more contiguous nucleotides (or any range derivable therein) of nucleic acid disclosed in this application, including, but not limited to SEQ ID NO:1, intron 5 of the mouse s-ship gene, an s-ship promoter, and any other SEQ ID NOs such as SEQ ID NO:2, SEQ ID NO:3, SEQ ID NO:4, SEQ ID NO:5, or any of SEQ ID NO:6-11 and/or 12-25.

The various probes and primers designed around the nucleotide sequences of the present invention may be of any length. By assigning numeric values to a sequence, for example, the first residue is 1, the second residue is 2, etc., an algorithm defining all primers can be proposed:
n to n+y
   where n is an integer from 1 to the last number of the sequence and y is the length of the primer minus one, where n+y does not exceed the last number of the sequence. Thus, for a 10-mer, the probes correspond to bases 1 to 10, 2 to 11, 3 to 12 . . . and so on. For a 15-mer, the probes correspond to bases 1 to 15, 2 to 16, 3 to 17 . . . and so on. For a 20-mer, the probes correspond to bases 1 to 20, 2 to 21, 3 to 22 . . . and so on.

It also will be understood that this invention is not limited to the particular nucleic acid sequences of SEQ ID NO:1. Recombinant vectors and isolated DNA segments may therefore variously include coding regions, coding regions bearing selected alterations or modifications in the basic coding region, or they may encode biologically functional equivalent sequences. For example, mutations can be made to SEQ ID NO:1-25 that potentially enhance or alter function relative to the native sequence or alternatively, may be silent with regard to function.

The s-ship promoter sequence of the invention is exemplified by the nucleic acid sequence given in SEQ ID NO:1. Alternatively, an s-ship promoter sequence can include all or part of SEQ ID NO:2, SEQ ID NO:3, SEQ ID NO:4 and/or SEQ ID NO:5, as well as any of SEQ ID NO:6-11, or any sequence with at least 80% identity to such sequences and/or capable of hyrbidizing to the complements of such sequences under conditions of high stringency. The invention is not limited to SEQ ID NO:1 as a person of ordinary skill in the art could readily manipulate SEQ ID NO:1 or use all or part of it in subsequent assays and experiments. In certain embodiments, the present invention concerns nucleic acid sequences capable of hybridizing all or parts of SEQ ID NO:1. Parts of SEQ ID NO:1 include, in specific embodiments, a s-ship1 promoter with one or more of the following regions of SEQ ID NO:1: from nucleotide (nt) 49485 to 60914 (11.5 kb-GFP construct); 49485 to 57072, which is 7588 nt (7.6 kb construct); from nt 49485 to 55810, which is 6326 nt (6.3 kb construct); from 49485 to 54755, but lacking 57050 to 57883 (6.2 kb-GFP construct); from nt 51389 to 55810, which is 4421 nt (4.4 kb construct); from nt 52199 to 56423, which is 4224 nt (4.2 kb construct); from nt 53820 to 55810, which is 1990 nt (1.9 kb construct); from nt 54755 to 55810, which is 1055 nt (0.96 kb construct); or from nt 55668 to 55810, which is 142 nt (44 nt construct). It is specifically contemplated that nucleic acids of the invention include those capable of hybridizing to such regions or to subsets of such regions. Moreover, it is contemplated that those nucleic acids capable of hybridizing to such regions may be at least 80, 85, 90, 95, 96, 97, 98, 99% or more complementary to all or part of these regions of SEQ ID NOs:1-25.

SEQ ID NO:1 is one sequence for the ship1 gene. The structure of the gene based on SEQ ID NO:1 is as follows: exon 1 (1-300); exon 2 (4914-4977); exon 3 (44875-45025); exon 4 (47380-47551); exon 5 (49130-49271); exon 6 (55771-55858); exon 7 (69077-61057); exon 8 (61175-61246); exon 9 (63231-63354); exon 10 (71113-71219); exon 11 (74821-74923); exon 12 (76837-77033); exon 13 (77601-77718); exon 14 (78653-78749); exon 15 (79268-79403); exon 16 (80787-80894); exon 17 (81041-81129); exon 18 (82789-82870); exon 19 (85604-85693); exon 20 (87766-87879); exon 21 (89288-89370); exon 22 (90735-90822); exon 23 (91701-91850); exon 24 (92863-92959); exon 25 (94708-94983); exon 26 (97360-97953); and exon 27 (98991-100141). The respective introns lie between the exon sequences. In certain embodiments, the s-ship promoter comprises the region spanning intron 5 to intron 6 (inclusive) (referred to as “intron 5/6 region”) or sequences from that region. This region is in SEQ ID NO:5. It will be understood that there may be minor sequence differences between different isolates and clones. In such cases, a person of skill the art would recognize corresponding regions between different isolates, clones, and strains.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture. It is generally appreciated that conditions can be rendered more stringent by the addition of increasing amounts of form amide. For example, under highly stringent conditions, hybridization to filter-bound DNA may be carried out in 0.5 M NaHPO₄, 7% sodium dodecyl sulfate (SDS), 1 mM EDTA at 65° C., and washing in 0.1×SSC/0.1% SDS at 68° C. (Ausubel et al., 1989).

Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Under low stringent conditions, such as moderately stringent conditions the washing may be carried out for example in 0.2×SSC/0.1% SDS at 42° C. (Ausubel et al, 1989). Hybridization conditions can be readily manipulated depending on the desired results.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

However, in addition to the unmodified s-ship promoter sequence of SEQ ID NO:1, the current invention includes derivatives of this sequence and compositions made therefrom. In particular, the present disclosure provides the teaching for one of skill in the art to make and use derivatives of the s-ship promoter. For example, the disclosure provides the teaching for one of skill in the art to delimit the functional elements within the s-ship promoter and to delete any non-essential elements. Functional elements also could be modified to increase the utility of the sequences of the invention for any particular application. For example, a functional region within the s-ship promoter of the invention could be modified to cause or increase tissue-specific expression. Such changes could be made by site-specific mutagenesis techniques, for example, as described below.

One efficient means for preparing such derivatives comprises introducing mutations into the sequences of the invention, for example, the sequence given in SEQ ID NO:1. Such mutants may potentially have enhanced or altered function relative to the native sequence or alternatively, may be silent with regard to function. It will be understood generally that any embodiment discussed in the application with respect to SEQ ID NO:1 may be applied with respect to any other SEQ ID NO, and vice versa.

Mutagenesis may be carried out at random and the mutagenized sequences screened for function in a trial-by-error procedure. Alternatively, particular sequences that provide the s-ship promoter with desirable expression characteristics could be identified and these or similar sequences introduced into other related or non-related sequences via mutation. Similarly, non-essential elements may be deleted without significantly altering the function of the elements. It further is contemplated that one could mutagenize these sequences in order to enhance their utility in expressing transgenes in a particular cell type, for example, in a particular stem cell.

The means for mutagenizing a DNA segment containing an s-ship promoter sequence of the current invention are well-known to those of skill in the art. Mutagenesis may be performed in accordance with any of the techniques known in the art, such as, but not limited to, synthesizing an oligonucleotide having one or more mutations within the sequence of a particular regulatory region. In particular, site-specific mutagenesis is a technique useful in the preparation of promoter mutants, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids also are routinely employed in site directed mutagenesis which eliminates the step of transferring the gene of interest from a plasmid to a phage.

Site-directed mutagenesis in accordance herewith typically is performed by first obtaining a single-stranded vector or melting apart of two strands of a double-stranded vector which includes within its sequence a DNA sequence that includes the s-ship promoter. An oligonucleotide primer bearing the desired mutated sequence is prepared, generally synthetically. This primer is then annealed with the single-stranded vector, and subjected to DNA polymerizing enzymes such as the E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and cells are selected which include recombinant vectors bearing the mutated sequence arrangement. Vector DNA can then be isolated from these cells and used for plant transformation. A genetic selection scheme was devised by Kunkel et al. (1987) to enrich for clones incorporating mutagenic oligonucleotides. Alternatively, the use of PCR™ with commercially available thermostable enzymes such as faq polymerase may be used to incorporate a mutagenic oligonucleotide primer into an amplified DNA fragment that can then be cloned into an appropriate cloning or expression vector. The PCR™-mediated mutagenesis procedures of Tomic et al. (1990) and Upender et al (1995) provide two examples of such protocols. A PCR™ employing a thermostable ligase in addition to a thermostable polymerase also may be used to incorporate a phosphorylated mutagenic oligonucleotide into an amplified DNA fragment that may then be cloned into an appropriate cloning or expression vector.

The preparation of sequence variants of the selected promoter DNA segments using site-directed mutagenesis is provided as a means of producing potentially useful promoter sequences and is not meant to be limiting as there are other ways in which sequence variants of DNA sequences may be obtained. For example, recombinant vectors encoding the desired promoter sequence may be treated with mutagenic agents, such as hydroxylamine, to obtain sequence variants.

As used herein, the term “oligonucleotide-directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” also is intended to refer to a process that involves the template-dependent extension of a primer molecule. The term template-dependent process refers to nucleic acid synthesis of an RNA or a DNA molecule wherein the sequence of the newly synthesized strand of nucleic acid is dictated by the well-known rules of complementary base pairing (see, for example, Watson and Ramstad, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety. A number of template dependent processes are available to amplify the target sequences of interest present in a sample, such methods being well known in the art and specifically disclosed herein below.

One efficient, targeted means for preparing mutagenized promoters or enhancers relies upon the identification of putative regulatory elements within the target sequence. This can be initiated by comparison with, for example, promoter sequences known to be expressed in a similar manner. Sequences which are shared among elements with similar functions or expression patterns are likely candidates for the binding of transcription factors and are thus likely elements which confer expression patterns. Confirmation of these putative regulatory elements can be achieved by deletion analysis of each putative regulatory region followed by functional analysis of each deletion construct by assay of a reporter gene which is functionally attached to each construct. As such, once a starting promoter or intron sequence is provided, any of a number of different functional deletion mutants of the starting sequence could be readily prepared.

As indicated above, deletion mutants of the s-ship promoter also could be randomly prepared and then assayed. With this strategy, a series of constructs are prepared, each containing a different portion of the clone (a subclone), and these constructs are then screened for activity. A suitable means for screening for activity is to attach a deleted promoter construct to a selectable or screenable marker, and to isolate only those cells expressing the marker protein. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous protein.

1. Vectors

Promoter sequences of the invention may be comprised in a vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., (1989) and Ausubel et al, 1996, both incorporated herein by reference. In addition to encoding a polypeptide, a vector may encode other polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA molecules or miRNA molecules. In addition to s-ship promoter regions, expression vectors can contain a variety of other “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various manmalian cells (Friedmann, 1989; Ridgeway, 1988; Baichwal and Sugden, 1986; Coupar et al., 1988; Horwich et al., 1990).

a. Promoters and Enhancers

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In addition to the s-ship promoter, other elements/promoters may be employed, in the context of the present invention, to regulate the expression of a gene. Table 1 is a list of other promoters and enhancers that may be used in conjunction with the s-ship promoter of the invention; this list also identifies references that indicate how promoters can be evaluated. It is not intended to be exhaustive of all the possible elements involved in the promotion of expression but, merely, to be exemplary thereof. Table 2 provides examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell et al., 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Chol et |
| | al., 1988; Reisman et al., 1989 |
| Papilloma Virus | Campo et al., 1983; Lusky et al., 1983; Spandidos |
| | and Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe et |
| | al., 1988; Rosen et al., 1988; Berkhout et al., 1989; |
| | Laspia et al., 1989; Sharp et al., 1989; Braddock et |
| | al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary tumor | | al., 1981; Majors et al., |
| virus) | | 1983; Chandler et al., 1983; |
| | | Lee et al., 1984; Ponta et |
| | | al., 1985; Sakai et al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996), and the SM22a promoter.

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, incorporated herein by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers.

In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk), chloramphenicol acetyltransferase (CAT), or luciferase may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

2. Heterologous Sequences

It is contemplated that polynucleotides of the invention include an s-ship promoter region controlling the expression of a heterologous nucleic acid sequence. The sequence may be a gene, cDNA sequence or an untranslated sequence, such as an siRNA. The invention is not limited to any specific sequence, but in certain embodiments, the heterologous sequence encodes any of the following proteins or RNAs.

Table 3 below provides different classes of proteins, and in some cases, examples of those proteins.

TABLE 3

| | Protein | | |
|---|---|---|---|
| Protein Genus | Subgenus | Protein Species | Protein Subspecies |
| 1) Toxins | Ribosome | | |
| | Inhibitory | | |
| | Proteins | | |
| | | Gelonin | |
| | | Ricin A Chain | |
| | | Pseudomonas | |
| | | Exotoxin | |
| | | Diptheria Toxin | |
| | | Mitogillin | |
| | | Saporin | |
| 2)Cytokines/Growth | Interleukins | IL-1, IL-2, IL-3, IL- | |
| Factors | | 4, IL-5, IL-6, IL-7, | |
| | | IL-8, IL-9, IL-10, IL- | |
| | | 11, IL-12, IL-13, IL- | |
| | | 14, IL-15, IL-16, IL- | |
| | | 17, IL-18, IL-19 | |
| | TNF | | |
| | LT | | |
| | Interferons | IFNα, IFNβ, IFNγ | |
| | Colony | GM-CSF, G-CSF, M- | |
| | Stimulating | CSF, CSF | |
| | Factors | | |
| | LIF | | |
| | Fibroblast | bFGF, FGF, FGF-1, | |
| | Growth Factors | FGF-2, FGF-3, FGF- | |
| | | 4, FGF-8, FGF-9, | |
| | | FGF-10, FGF-18, | |
| | | FGF-20, FGF, 23 | |
| | VEGF | | |
| 3) Enzymes | Oxidoreductases | | |
| | Transferases | Transferring one- | Methyltransferases |
| | | carbon groups | |
| | | | Carboxyl and |
| | | | carbamoyltransferases |
| | | | Amidinotransferases |
| | | Transferring | |
| | | aldehyde or ketone | |
| | | residues | |
| | | Acyltransferases | Acyltransferases |
| | | | Aminoacyltransferases |
| | | Glycosyltransferases | Hexosyltransferases |
| | | Transferring alkyl or | |
| | | aryl groups, other | |
| | | than methyl groups | |
| | | Transferring | Transaminases |
| | | nitrogenous groups | |
| | | | Oximinotransferases |
| | | Transferring | Phosphotransferases |
| | | phosphorous- | |
| | | containing groups | |
| | | | Diphosphotransferases |
| | | | Nucleotidyltransferases |
| | | Transferring sulfur- | Sulfur-transferases |
| | | containing groups | |
| | | | Sulfotransferases |
| | | | CoA-transferases |
| | | Transferring | |
| | | selenium-containing | |
| | | groups | |
| | Hydrolases | Acting on ester bonds | |
| | | Glycosylases | |
| | | Acting on ether | |
| | | bonds | |
| | | Acting on peptide | |
| | | bonds (peptide | |
| | | hydrolases) | |
| | | Acting on carbon- | |
| | | nitrogen bonds, other | |
| | | than peptide bonds | |
| | | Acting on acid | |
| | | anhydrides | |
| | | Acting on carbon- | |
| | | carbon bonds. | |
| | | Acting on halide | |
| | | bonds | |
| | | Acting on | |
| | | phosphorus-nitrogen | |
| | | bonds. | |
| | | Acting on sulfur- | |
| | | nitrogen bonds | |
| | | Acting on carbon- | |
| | | phosphorus bonds | |
| | | Acting on sulfur- | |
| | | sulfur bonds | |
| | Lyases | Carbon-carbon | |
| | | lyases. | |
| | | Carbon-oxygen | |
| | | lyases | |
| | | Carbon-nitrogen | |
| | | lyases | |
| | | Carbon-sulfur lyases | |
| | | Carbon-halide lyases | |
| | | Phosphorus-oxygen | |
| | | lyases | |
| | Isomerases | Racemases and | |
| | | epimerases | |
| | | Cis-trans-isomerases | |
| | | Intramolecular | |
| | | oxidoreductases | |
| | | Intromolecular | |
| | | transferases | |
| | | (mutases) | |
| | | Phosphotransferases | |
| | | (phosphomutases) | |
| | Ligases | Forming carbon- | |
| | | oxygen bonds | |
| | | Forming carbon- | |
| | | sulfur bonds | |
| | | Forming carbon- | |
| | | nitrogen bonds. | |
| | | Forming carbon- | |
| | | carbon bonds | |
| | | Forming phosphoric | |
| | | ester bonds | |

Other examples include but are not limited to the following:

a. Cytokines

Another class of compounds that is contemplated to be operatively linked to a therapeutic polypeptide, such as a toxin, includes interleukins and cytokines, such as interleukin 1 (IL-1), IL-2, IL-3, IL-4, IL-5, IL-6, IL-7, IL-8, IL-9, IL-10, IL-11, IL-12, IL-13, IL-14, IL-15, β-interferon, α-interferon, γ-interferon, angiostatin, thrombospondin, endostatin, METH-1, METH-2, Flk2/Flt3 ligand, GM-CSF, G-CSF, M-CSF, and tumor necrosis factor (TNF).

c. Growth Factors

In other embodiments of the present invention, growth factors or ligands can be complexed with the therapeutic agent. Examples include VEGF/VPF, FGF, TGFβ, ligands that bind to a TIE, tumor-associated fibronectin isoforms, scatter factor, hepatocyte growth factor, fibroblast growth factor, platelet factor (PF4), PDGF, KIT ligand (KL), colony stimulating factors (CSFs), LIF, and TIMP.

d. Inducers of Cellular Proliferation

Another group of proteins that may be used in conjunction with modified proteins of the present invention, such as modified gelonin toxin, comprises proteins that induce cellular proliferation. In some embodiments, the toxin is operatively linked to a ribozyme that can inactivate an inducer of cellular proliferation, while in others, the toxin is linked to the inducer itself. Alternatively, a toxin may be attached to an antibody that recognizes an inducer of cell proliferation.

The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

e. Inhibitors of Cellular Proliferation

The tumor suppressors function to inhibit excessive cellular proliferation. The inactivation of these genes destroys their inhibitory activity, resulting in unregulated proliferation. It is contemplated that toxins may be attached to antibodies that recognize mutant tumor suppressors or wild-type tumor suppressors. Alternatively, a toxin may be linked to all or part of the tumor suppressor. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, mda-7, fus-1, FHIT, p16, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

Other examples are provided in Table 4 below.

f. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al, 1985; Tsujimoto and Croce, 1986). The evolutionarily conserved Bcl-2 protein now is recognized to be a member of a family of related proteins, which can be categorized as death agonists or death antagonists.

Apo2 ligand (Apo2L, also called TRAIL) is a member of the tumor necrosis factor (TNF) cytokine family. TRAIL activates rapid apoptosis in many types of cancer cells, yet is not toxic to normal cells. TRAIL mRNA occurs in a wide variety of tissues. Most normal cells appear to be resistant to TRAIL's cytotoxic action, suggesting the existence of mechanisms that can protect against apoptosis induction by TRAIL. The first receptor described for TRAIL, called death receptor 4 (DR4), contains a cytoplasmic “death domain”; DR4 transmits the apoptosis signal carried by TRAIL. Additional receptors have been identified that bind to TRAIL. One receptor, called DR5, contains a cytoplasmic death domain and signals apoptosis much like DR4. The DR4 and DR5 mRNAs are expressed in many normal tissues and tumor cell lines. Recently, decoy receptors such as DcR1 and DcR2 have been identified that prevent TRAIL from inducing apoptosis through DR4 and DR5. These decoy receptors thus represent a novel mechanism for regulating sensitivity to a pro-apoptotic cytokine directly at the cell's surface. The preferential expression of these inhibitory receptors in normal tissues suggests that TRAIL may be useful as an anticancer agent that induces apoptosis in cancer cells while sparing normal cells. (Marsters et al 1999).

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri). It is contemplated that any of these polypeptides, including TRAIL, or any other polypeptides that induce or promote of apoptosis, may be operatively linked to a toxin, or that an antibody recognizing any of these polypeptides may also be attached to a toxin.

Granzyme enzymes are also capable of inducing apoptosis. These include Granzyme A and Granzyme B.

Other examples are provided in Table 4 below.

TABLE 4

| | | | |
|---|---|---|---|
| Gene | Source | Human Disease | Function |

Growth Factors

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma virus | PDGF B |

Receptor Tyrosine Kinases

| | | | |
|---|---|---|---|
| ERBB/HER | Avian erythroblastosis | Amplified, deleted | EGF/TGF-α/ |
| | virus; ALV promoter | Squamous cell | Amphiregulin/ |
| | insertion; amplified | Cancer; glioblastoma | Hetacellulin receptor |
| | human tumors | | |
| ERBB-2/NEU/HER-2 | Transfected from rat | Amplified breast, | Regulated by NDF/ |
| | Glioblastomas | Ovarian, gastric | Heregulin and EGF- |
| | | cancers | Related factors |
| FMS | SM feline sarcoma virus | | CSF-1 receptor |
| KIT | HZ feline sarcoma virus | | MGF/Steel receptor |
| | | | Hematopoieis |
| TRK | Transfection from | | NGF (nerve growth |
| | human colon cancer | | Factor) receptor |
| MET | Transfection from | | Scatter factor/HGF |
| | human osteosarcoma | | Receptor |
| RET | Translocations and point | Sporadic thyroid cancer; | Orphan receptor Tyr |
| | mutations | familial medullary | Kinase |
| | | thyroid cancer; | |
| | | multiple endocrine | |
| | | neoplasias 2A and 2B | |
| ROS | URII avian sarcoma | | Orphan receptor Tyr |
| | Virus | | Kinase |
| PDGF receptor | Translocation | Chronic | TEL(ETS-like |
| | | Myelomonocytic | transcription factor)/ |
| | | Leukemia | PDGF receptor gene |
| | | | Fusion |
| TGF-β receptor | | Colon carcinoma | |
| | | mismatch mutation | |
| | | target | |

NONRECEPTOR TYROSINE KINASES

| | | | |
|---|---|---|---|
| ABI. | Abelson Mul.V | Chronic myelogenous | Interact with RB, RNA |
| | | leukemia translocation | polymerase, CRK, |
| | | with BCR | CBL |
| FPS/FES | Avian Fujinami SV; GA | | |
| | FeSV | | |
| LCK | Mul.V (murine leukemia | | Src family; T cell |
| | virus) promoter | | signaling; interacts |
| | insertion | | CD4/CD8 T cells |
| SRC | Avian Rous sarcoma | | Membrane-associated |
| | Virus | | Tyr kinase with |
| | | | signaling function; |
| | | | activated by receptor |
| | | | kinases |
| YES | Avian Y73 virus | | Src family; signaling |

SER/THR PROTEIN KINASES

| | | |
|---|---|---|
| AKT | AKT8 murine retrovirus | Regulated by PI(3)K?; |
| | | regulate 70-kd S6 k? |
| MOS | Maloney murine SV | GVBD; cystostatic |
| | | factor; MAP kinase |
| | | kinase |
| PIM-1 | Promoter insertion | |
| | Mouse | |
| RAF/MIL | 3611 murine SV; MH2 | Signaling in RAS |
| | avian SV | Pathway |

MISCELLANEOUS CELL SURFACE

| | | | |
|---|---|---|---|
| APC | Tumor suppressor | Colon cancer | Interacts with catenins |
| DCC | Tumor suppressor | Colon cancer | CAM domains |
| E-cadherin | Candidate tumor | Breast cancer | Extracellular homotypic |
| | Suppressor | | binding; intracellular |
| | | | interacts with catenins |
| PTC/NBCCS | Tumor suppressor and | Nevoid basal cell cancer | 12 transmembrane |
| | Drosophilia homology | syndrome (Gorline | domain; signals |
| | | syndrome) | through Gli homogue |
| | | | CI to antagonize |
| | | | hedgehog pathway |
| TAN-1 Notch | Translocation | T-ALI. | Signaling |
| homologue | | | |

MISCELLANEOUS SIGNALING

| | | | |
|---|---|---|---|
| BCL-2 | Translocation | B-cell lymphoma | Apoptosis |
| CBL | Mu Cas NS-1 V | | Tyrosine- |
| | | | Phosphorylated RING |
| | | | finger interact Ab1 |
| CRK | CT1010 ASV | | Adapted SH2/SH3 |
| | | | interact Ab1 |
| DPC4 | Tumor suppressor | Pancreatic cancer | TGF-β-related signaling |
| | | | Pathway |
| MAS | Transfection and | | Possible angiotensin |
| | Tumorigenicity | | Receptor |
| NCK | | | Adaptor SH2/SH3 |

GUANINE NUCLEOTIDE EXCHANGERS AND BINDING PROTEINS

| | | | |
|---|---|---|---|
| BCR | | Translocated with ABL | Exchanger; protein |
| | | in CML | Kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N-RAS | HaRat SV; Ki RaSV; | Point mutations in many | Signal cascade |
| | Balb-MoMuSV; | human tumors | |
| | Transfection | | |
| VAV | Transfection | | S112/S113; exchanger |

NUCLEAR PROTEINS AND TRANSCRIPTION FACTORS

| | | | |
|---|---|---|---|
| BRCA1 | Heritable suppressor | Mammary | Localization unsettled |
| | | cancer/ovarian cancer | |
| BRCA2 | Heritable suppressor | Mammary cancer | Function unknown |
| ERBA | Avian erythroblastosis | | Thyroid hormone |
| | Virus | | receptor (transcription) |
| ETS | Avian E26 virus | | DNA binding |
| EVII | MuLV promotor | AML | Transcription factor |
| | Insertion | | |
| FOS | FBI/FBR murine | | Transcription factor |
| | osteosarcoma viruses | | with c-JUN |
| GLI | Amplified glioma | Glioma | Zinc finger; cubitus |
| | | | interruptus homologue |
| | | | is in hedgehog |
| | | | signaling pathway; |
| | | | inhibitory link PTC |
| | | | and hedgehog |
| HMGI/LIM | Translocation t(3:12) | Lipoma | Gene fusions high |
| | t(12:15) | | mobility group |
| | | | HMGI-C (XT-hook) |
| | | | and transcription factor |
| | | | LIM or acidic domain |
| JUN | ASV-17 | | Transcription factor |
| | | | AP-1 with FOS |
| MLL/VHRX + ELI/MEN | Translocation/fusion | Acute myeloid leukemia | Gene fusion of DNA- |
| | ELL with MLL | | binding and methyl |
| | Trithorax-like gene | | transferase MLL with |
| | | | ELI RNA pol II |
| | | | elongation factor |
| MYB | Avian myeloblastosis | | DNA binding |
| | Virus | | |
| MYC | Avian MC29; | Burkitt's lymphoma | DNA binding with |
| | Translocation B-cell | | MAX partner; cyclin |
| | Lymphomas; promoter | | regulation; interact |
| | Insertion avian | | RB?; regulate |
| | leukosis | | apoptosis? |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator or |
| | | syndrome | elongin; transcriptional |
| | | | elongation complex |
| WT-1 | | Wilm's tumor | Transcription factor |

CELL CYCLE/DNA DAMAGE RESPONSE

| | | | |
|---|---|---|---|
| ATM | Hereditary disorder | Ataxia-telangiectasia | Protein/lipid kinase |
| | | | homology; DNA |
| | | | damage response |
| | | | upstream in P53 |
| | | | pathway |
| BCL-2 | Translocation | Follicular lymphoma | Apoptosis |
| FACC | Point mutation | Fanconi's anemia group | |
| | | C (predisposition | |
| | | leukemia | |
| FHIT | Fragile site 3p14.2 | Lung carcinoma | Histidine triad-related |
| | | | diadenosine 5′,3″″- |
| | | | P¹.p⁴ tetraphosphate |
| | | | asymmetric hydrolase |
| hMLI/MutL | | HNPCC | Mismatch repair; MutL |
| | | | Homologue |
| HMSH2/MutS | | HNPCC | Mismatch repair; MutS |
| | | | Homologue |
| HPMS1 | | HNPCC | Mismatch repair; MutL |
| | | | Homologue |
| hPMS2 | | HNPCC | Mismatch repair; MutL |
| | | | Homologue |
| INK4/MTS1 | Adjacent INK-4B at | Candidate MTS1 | p16 CDK inhibitor |
| | 9p21; CDK complexes | suppressor and MLM | |
| | | melanoma gene | |
| INK4B/MTS2 | | Candidate suppressor | p15 CDK inhibitor |
| MDM-2 | Amplified | Sarcoma | Negative regulator p53 |
| p53 | Association with SV40 | Mutated >50% human | Transcription factor; |
| | T antigen | tumors, including | checkpoint control; |
| | | hereditary Li-Fraumeni | apoptosis |
| | | syndrome | |
| PRAD1/BCL1 | Translocation with | Parathyroid adenoma; | Cyclin D |
| | Parathyroid hormone | B-CLL | |
| | or IgG | | |
| RB | Hereditary | Retinoblastoma; | Interact cyclin/cdk; |
| | Retinoblastoma; | osteosarcoma; breast | regulate E2F |
| | Association with many | cancer; other sporadic | transcription factor |
| | DNA virus tumor | cancers | |
| | Antigens | | |
| XPA | | xeroderma | Excision repair; photo- |
| | | pigmentosum; skin | product recognition; |
| | | cancer predisposition | zinc finger |

As discussed above, other heterologous sequences include those that can be used as a reporter, such as a screenable or selectable marker. Included in this category are colorimetric or fluorescent reporters (GFP, β-gal, etc.), enzymatic reporters (CAT, luciverase, horseradish peroxidase, etc.), or drug resistance reporters.

Furthermore, it is contemplated that encoded sequences may be fusion proteins, fragments or portions of proteins (including peptides), chimeric proteins, as well as non-protein molecules such as functional RNA molecules (tRNA, rRNA, miRNA, siRNA, antisense, ribozyme, etc.). Such sequences are well known to those of skill in the art.

Moreover, in some embodiments the nucleic acid sequence is a cell-surface marker or an antibody. Cell-surface molecules include, but are not limited to, cluster of differentiation antigens (CD), CTLA, CMRF, cellular adhesion molecules (CAM) molecules such as CD4, CD8, CMRF83, CTLA4, etc. In some embodiments of the invention, these molecules are used to identify cells with stem-cell properties. For example, the cell-surface molecule can be used to identify and/or segregate such cells. FACS analysis can be implemented for this purpose.

B. Host Cells

The invention include host cells transfected, transformed, or infected with a recombinant nucleic acid sequence discussed in this application. Such host cells would be considered recombinant host cells. The mode of transmission of the nucleic acid sequence into the host cell is of no significant consequence with respect to the invention; therefore, the terms “transfected,” “transformed,” and “infected: are used interchangeably unless otherwise specified.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid, such as an s-ship promoter sequence, is transferred or introduced into the host cell. A transformed cell includes the primary subject cell and its progeny.

Host cells may be derived from prokaryotes or eukaryotes, including yeast cells, insect cells, and mammalian cells, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (World Wide Web at atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla, Calif.). Alternatively, bacterial cells such as E. coli LE392 could be used as host cells for phage viruses. Appropriate yeast cells include Saccharomyces cerevisiae, Saccharomyces pombe, and Pichia pastoris.

Examples of eukaryotic host cells for replication and/or expression of a vector include HeLa, NIH3T3, Jurkat, 293, Cos, CHO, Saos, and PC12. Stem cell lines and other immature cell lines are specifically contemplated as suitable host cells of the invention. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In certain embodiments of the invention, a host cell refers to a cell obtained from a subject that is transfected, transformed, or infected with an s-ship promoter region. The promoter region may integrate into the cell's genome or it may exist in the cell extrachromosomally. Moreover, it may be operably linked to a nucleic acid sequence whose expression is controlled by the s-ship promoter region. In further embodiments, the nucleic acid sequence is a heterologous sequence, meaning it is not one associated with an s-ship promoter in nature. In some cases, the heterologous sequence is a reporter gene. In other cases, it is a therapeutic or diagnostic nucleic acid, meaning that the resulting transcript or protein can be used as a therapeutic or diagnostic with respect to the host cell. It is contemplated that the host cell may be obtained from subject, transfected or infected with the s-ship promoter region, which may or may be operably linked to a therapeutic or diagnostic nucleic acid, and returned back to the subject. This ex vivo approach can be used in a variety of contexts, including but not limited to, the treatment of cancer or other hyperproliferative diseases or disorders, autoimmune disease, diseases or disorders involving stem cells, diseases or disorders treatable with stem cells, and/or diseases or disorders caused by protein deficiencies.

In certain embodiments, the s-ship promoter regions are employed to direct transcription in cells in a temporal or developmentally specific manner. Therefore, it is contemplated that in some embodiments of the invention, a recombinant host cell contains a heterologous nucleic acid sequence under the control of an s-ship promoter region and the heterologous nucleic acid sequence is initially expressed but after the cell differentiates, expression is limited or eliminated. In some embodiments, stem cells are used for ex vivo therapy in which stem cells or a subset of progenitor cells are obtained either from a non-recipient donor or from the recipient themselves, introduced with the s-ship promoter-heterologous nucleic acid sequence, and then administered to the subject in which therapy is needed. It is contemplated that the introduced cells could potentially provide expression insofar as they did not become differentiated.

The invention has applicability also with respect to tumor cells as host cells for nucleotides containing s-ship promoter regions. The idea of tumor stem cells has been around for more than 40 years, however, more recent technology has given further credible support to this concept. Initially, experiments demonstrated that about one in a million tumor cells can initiate a tumor upon transplantation into autologous hosts.

Two models were proposed to account for these findings; 1) all tumor cells are alike and stochastic events determine whether a cell might be a tumor initiating cell, and 2) not all tumor cells are alike, but a very few (about 1/10⁶ tumor cells) are inherently capable of tumor initiation on transplantation into a suitable host. These models, termed variously: stochastic vs. hierarchy, nurture vs. nature, probabilistic vs. deterministic, have been applied to understanding many stem/developmental systems. Recently, however, these models of tumor development have been tested using the more refined NOD/SCID mouse for transplantation. Breast tumor-specific cell-surface markers (along with the absence of lineage markers (lin−) of differentiated cells) have been used for identification and isolation of the few tumor initiating cells within the breast tumor mass (Al-Hajj M. Wicha M S. Benito-Hernandez A. Morrison S J. Clarke M F. Prospective identification of tumorigenic breast cancer cells. Proc Natl Acad Sci USA. 100:3983-8, 2003.). In this case, specific populations capable of tumor transplantation were identified, and these tumor cells were all lineage-minus. Further experiments showed that the same tumor population could be isolated from the transplanted animals, and that the transplanted cells had reestablished the same heterogeneity in transplanted tumor as found in the primary population. These data strongly support the hierarchy model (#2, above) in which specific cells are destined for sustaining the tumorigenic capability. The results above were obtained with breast tumors, but studies in both brain and acute myeloid leukemia (AML) have supported the same characteristics of tumor cells able to initiate tumors on transplantation (Lapidot et al., 1994; Singh et al., 2003; Bhatia et al, 1997).

In the case of human AML, hematopoietic stem cell surface markers are well known (CD34+, CD38−. Lin−, Thy1.1+, c-Kitlo) and this “stem cell” fraction contained the tumor-initiating cells. Results from all three tumor systems examined indicate that a tissue stem cell might be the initial target for tumor formation. The properties of the tumor stem cells identified by transplantation suggest that they sustain the tumor mass by self-replication; however, partial differentiation of the tumor stem cell produces a vast majority of tumor cells, which can no longer sustain the tumor on transplantation. Thus, like normal stem cells, tumor stem cells self-replicate and differentiate producing a larger mass of differentiated but non-transplantable tumor cells.

This theory of tumor stem cells as the primary source of tumor growth has important implications for tumor therapy and potential stem cell therapies for correction of tissue-dependent human diseases. The fact that the s-SHIP promoter expresses exclusively in stem/progenitors in the embryo and adult suggests s-SHIP protein can be used to further study tumor models and evaluate pathways and to drive expression of agents, including therapeutic and diagnostic agents, in tumor cells, particularly those that qualify as tumor stem cells.

C. Assays of Transgene Expression

Assays may be employed with the instant invention for determination of the relative efficiency of transgene expression. For example, assays may be used to determine the efficacy of deletion mutants of the s-ship promoter in directing expression of exogenous proteins. Similarly, one could produce random or site-specific mutants of the s-ship promoter of the invention and assay the efficacy of the mutants in the expression of a given transgene. Alternatively, assays could be used to determine the efficacy of the s-ship promoter in directing protein expression when used in conjunction with various different enhancers, terminators or other types of elements potentially used in the preparation of transformation constructs.

For mammals, expression assays may comprise a system utilizing cell lines, or alternatively, whole organisms. Additionally, assays of tissue or developmental specific promoters are generally feasible.

The biological sample to be assayed may comprise nucleic acids isolated from the cells of any plant material according to standard methodologies (Sambrook et al., 1989). The nucleic acid may be genomic DNA or fractionated or whole cell RNA. Where RNA is used, it may be desired to convert the RNA to a complementary DNA. In one embodiment of the invention, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given sample with a statistically significant reference group of non-transformed control cells. Typically, the non-transformed control cells will be of a genetic background similar to the transformed cells. In this way, it is possible to detect differences in the amount or kind of protein detected in various transformed cells.

As indicated, a variety of different assays are contemplated in the screening of cells or animals of the current invention and associated promoters. These techniques may in cases be used to detect for both the presence and expression of the particular genes as well as rearrangements that may have occurred in the gene construct. The techniques include but are not limited to, fluorescent in situ hybridization (FISH), direct DNA sequencing, pulsed field gel electrophoresis (PFGE) analysis, Southern or Northern blotting, single-stranded conformation analysis (SSCA), RNAse protection assay, allele-specific oligonucleotide (ASO), dot blot analysis, denaturing gradient gel electrophoresis, RFLP and PCR™-SSCP.

1. Quantitation of Gene Expression with Relative Quantitative RT-PCR™

Reverse transcription (RT) of RNA to cDNA followed by relative quantitative PCR™ (RT-PCR™) can be used to determine the relative concentrations of specific mRNA species, for example, an mRNA whose expression is controlled by an s-ship promoter. By determining that the concentration of a specific mRNA species varies, it can be shown that the gene encoding the specific mRNA species is differentially expressed. In this way, a promoters expression profile can be rapidly identified, as can the efficacy with which the promoter directs transgene expression.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ study to successfully determine the relative abundances of a particular mRNA species is that relative concentrations of the amplifiable cDNAs must be normalized to some independent standard. The goal of an RT-PCR™ study is to determine the abundance of a particular mRNA species relative to the average abundance of all mRNA species in the sample.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for plant tissue. The problems inherent in plant tissue samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

2. Marker Gene Expression

Marker genes represent an efficient means for assaying the expression of transgenes. Using, for example, a selectable marker gene, one could quantitatively determine the expression levels in the cell using a construct comprising the selectable marker coding region operably linked to the promoter to be assayed, e.g., an s-ship promoter. Alternatively, particular cell types could be exposed to a selective agent and the relative resistance provided in these cells quantified, thereby providing an estimate of the tissue specific expression of the promoter.

Screenable markers constitute another efficient means for quantifying the expression of a given transgene. Potentially any screenable marker could be expressed and the marker gene product quantified, thereby providing an estimate of the efficiency with which the promoter directs expression of the transgene. Quantification can readily be carried out using either visual means, or, for example, a photon counting device.

A preferred screenable marker gene assay for use with the current invention include the use of the screenable marker gene β-galactosidase (β-gal), luciferase, or green fluorescent protein (GFP).

3. Purification and Assays of Proteins

One means for determining the efficiency with which a particular transgene is expressed is to purify and quantify a polypeptide expressed by the transgene. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; and isoelectric focusing. A particularly efficient method of purifying peptides is fast protein liquid chromatography or even HPLC.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide being assayed always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is well known to those of skill in the art.

D. Methods of Gene Transfer

Suitable methods for nucleic acid delivery to effect expression of compositions of the present invention are believed to include virtually any method by which a nucleic acid (e.g., DNA, including viral and nonviral vectors) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

E. Transgenic and Knockout Animals

1. Transgenic Animals

It is further contemplated that transgenic animals are part of the present invention. A transgenic animal of the present invention may involve an animal in which an s-ship promoter drives the expression of a transgene. The transgene can be expressed temporally or spatially in a manner different than or the same as a non-transgenic animal. The transgene may also be heterologous with respect to the host cell or organism, such as, for example, the luciferase gene in a mammalian cell. Moreover, it is contemplated that the transgene may be expressed in a different tissue type or in a different amount or at a different time than the endogenously expressed version of the transgene.

In a general aspect, a transgenic animal is produced by the integration of a given transgene into the genome in a manner that permits the expression of the transgene, or by disrupting the wild-type gene, leading to a knockout of the wild-type gene. Methods for producing transgenic animals are generally described by Wagner and Hoppe (U.S. Pat. No. 4,873,191; which is incorporated herein by reference), Brinster et al. (1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

U.S. Pat. No. 5,639,457 is also incorporated herein by reference to supplement the present teaching regarding transgenic pig and rabbit production. U.S. Pat. Nos. 5,175,384; 5,175,385; 5,530,179, 5,625,125, 5,612,486 and 5,565,186 are also each incorporated herein by reference to similarly supplement the present teaching regarding transgenic mouse and rat production. Transgenic animals may be crossed with other transgenic animals or knockout animals to evaluate phenotype based on compound alterations in the genome.

2. Knockout Animals or Cells

The generation of an animal model lacking s-ship or a particular nucleic acid (encoding an RNA that is translated or not) is contemplated as part of the present invention to understand further stem cell function. This strategy could also be implemented in cell culture as well.

The lack of activity as a result of the knockout may provoke various types of pathophysiological disturbances in a knockout animal or cell. This can be used to characterize the role or function of a particular gene product at a particular time in development or in a particular cell type. Use of the s-ship promoter can be used to drive the expression of the knockout gene such that only certain cells, for example stem cells, may be affected. One method of inhibiting the endogenous expression of a particular gene in an animal is to disrupt the gene in germline cells and produce offspring from these cells. This method is generally known as knockout technology. U.S. Pat. No. 5,616,491, incorporated herein by reference in its entirety, generally describes the techniques involved in the preparation of knockout mice, and in particular describes mice having a suppressed level of expression of the gene encoding CD28 on T cells, and mice wherein the expression of the gene encoding CD45 is suppressed on B cells. Pfeffer et al. (1993) describe mice in which the gene encoding the tumor necrosis factor receptor p55 has been suppressed. The mice showed a decreased response to tumor necrosis factor signaling. Fung-Leung et al. (1991a; 1991b) describe knockout mice lacking expression of the gene encoding CD8. These mice were found to have a decreased level of cytotoxic T cell response to various antigens and to certain viral pathogens such as lymphocytic choriomeningitis virus.

The term “knockout” refers to a partial or complete suppression of the expression of at least a portion of a protein encoded by an endogenous DNA sequence in a cell. The term “knockout construct” refers to a nucleic acid sequence that is designed to decrease or suppress expression of a protein encoded by endogenous DNA sequences in a cell. The nucleic acid sequence used as the knockout construct is typically comprised of: (1) DNA from some portion of the gene (exon sequence, intron sequence, and/or promoter sequence) to be suppressed, in conjunction with all or part of the s-ship promoter; and (2) a marker sequence used to detect the presence of the knockout construct in the cell. The knockout construct is inserted into a cell, and integrates with the genomic DNA of the cell in such a position so as to prevent or interrupt transcription of the native DNA sequence. Such insertion usually occurs by homologous recombination (i.e., regions of the knockout construct that are homologous to endogenous DNA sequences hybridize to each other when the knockout construct is inserted into the cell and recombine so that the knockout construct is incorporated into the corresponding position of the endogenous DNA).

The knockout construct nucleic acid sequence may comprise 1) a full or partial sequence of one or more exons and/or introns of the gene to be suppressed, 2) a full or partial promoter sequence of the gene to be suppressed, or 3) combinations thereof. Typically, the knockout construct is inserted into an embryonic stem cell (ES cell) and is integrated into the ES cell genomic DNA, usually by the process of homologous recombination. This ES cell is then injected into, and integrates with, the developing embryo.

The phenotype of a mouse heterozygous for the knockout may lend clues as to the function and importance of that gene or sequence, as well as contribute an understanding about its physiological relevance, particularly with respect to disease states. Animals completely lacking the targeted gene (homozygous null) may provide additional information. Mice lacking the targeted gene may not be viable, which itself is indicative of the importance of that gene. Should such mice be viable (heterozygous or homozygous nulls), they may be crossed with other transgenic or knockout mice. Furthermore, knock-out mice having any phenotype that resembles a disease state may be used to screen or test therapeutic drugs that slow, modify, or cure conditions. As is known to the skilled artisan, a conditional knockout, wherein the gene is disrupted under certain conditions, is frequently used.

3. Conditional Transgenic and Knockdown Animals and Cells

The present invention further contemplates conditional transgenic or knockdown animals (or cells in culture), such as those produced using recombination methods. Bacteriophage P1 Cre recombinase and flp recombinase from yeast plasmids are two non-limiting examples of site-specific DNA recombinase enzymes which cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present invention. Such recombinases include the Int recombinase of bacteriophage λ (with or without X is) (Weisberg et. al., 1983), herein incorporated by reference); TpnI and the β-lactamase transposons (Mercier et al., 1990); the Tn3 resolvase (Flanagan and Fennewald, 1989); the yeast recombinases (Matsuzaki et al, 1990); the B. subtilis SpoIVC recombinase (Sato et al., 1990); the Flp recombinase (Schwartz and Sadowski, 1989; Parsons et al., 1990; Golic and Lindquist, 1989; Amin et al., 1990); the Hin recombinase (Glasgow et al., 1989); immunoglobulin recombinases (Malynn et al., 1988); and the Cin recombinase (Haffiter and Bickle, 1988; Hubner et al., 1989), all herein incorporated by reference. Such systems are discussed (Echols, 1990; de Villartay, 1988; Craig, 1988; Poyart-Salmeron et al., 1989; Hunger-Bertling et al., 1990; and Cregg and Madden, 1989), all herein incorporated by reference.

Of particular interest in the present invention is the Cre recombinase. Cre has been purified to homogeneity, and its reaction with the loxP site has been extensively characterized (Abremski and Hess, 1984), herein incorporated by reference). Cre protein has a molecular weight of 35,000 and can be obtained commercially from New England Nuclear/DuPont. The cre gene (which encodes the Cre protein) has been cloned and expressed (Abremski et al., 1983), herein incorporated by reference). The Cre protein mediates recombination between two loxP sequences (Sternberg et al., 1981), which may be present on the same or different DNA molecule. Because the internal spacer sequence of the loxP site is asymmetrical, two loxP sites can exhibit directionality relative to one another (Hoess and Abremski, 1984). Thus, when two sites on the same DNA molecule are in a directly repeated orientation, Cre will excise the DNA between the sites (Abremski et al., 1983). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

Recombinases have important application for characterizing gene function in knockout models. When the constructs described herein are used to disrupt limulus clotting factor protease-like genes, a fusion transcript can be produced when insertion of the positive selection marker occurs downstream (3′) of the translation initiation site of the limulus clotting factor protease-like gene. The fusion transcript could result in some level of protein expression with unknown consequence. It has been suggested that insertion of a positive selection marker gene can affect the expression of nearby genes. These effects may make it difficult to determine gene function after a knockout event since one could not discern whether a given phenotype is associated with the inactivation of a gene, or the transcription of nearby genes. Both potential problems are solved by exploiting recombinase activity. When the positive selection marker is flanked by recombinase sites in the same orientation, the addition of the corresponding recombinase will result in the removal of the positive selection marker. In this way, effects caused by the positive selection marker or expression of fusion transcripts are avoided.

### III. PROTEINACEOUS COMPOSITIONS

In certain embodiments, the present invention concerns novel compositions comprising at least one proteinaceous molecule, such as s-SHIP1, SHIP1, or a modulator of an s-ship1 promoter. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible.

### IV. THERAPEUTIC APPLICATIONS

The invention is widely applicable to a variety of situations where it is desirable to be able to regulate the level of gene expression, such as by turning gene expression “on” and “off”, in a rapid, efficient and controlled manner without causing pleiotropic effects or cytotoxicity. The invention may be particularly useful for gene therapy purposes in humans, in treatments for either genetic or acquired diseases. The general approach of gene therapy involves the introduction of one or more nucleic acid molecules into cells such that one or more gene products encoded by the introduced genetic material are produced in the cells to restore or enhance a functional activity. For reviews on gene therapy approaches Anderson, et al. (1992; Miller et al. (1992); Friedmann et al. (1989); and Cournoyer et al. (1990). However, current gene therapy vectors typically utilize constitutive regulatory elements which are responsive to endogenous transcriptions factors. These vector systems do not allow for the ability to modulate the level of gene expression in a subject. In contrast, the regulatory system of the invention provides this ability.

To use the system of the invention for gene therapy purposes, at least one DNA molecule is introduced into cells of a subject in need of gene therapy (e.g., a human subject suffering from a genetic or acquired disease) to modify the cells. The cells are modified to comprise: 1) nucleic acid encoding an inducible regulator of the invention in a form suitable for expression of the inducible regulator in the host cells; and 2) an siRNA (e.g., for therapeutic purposes) operatively linked to a tissue-specific promoter such as an s-ship1 promoter. A single DNA molecule encoding components of the regulatory system of the invention can be used, or alternatively, separate DNA molecules encoding each component can be used. The cells of the subject can be modified ex vivo and then introduced into the subject or the cells can be directly modified in vivo by conventional techniques for introducing nucleic acid into cells. Thus, the regulatory system of the invention offers the advantage over constitutive regulatory systems of allowing for modulation of the level of gene expression depending upon the requirements of the therapeutic situation.

Genes of particular interest to be knocked down or knocked out in cells of a subject for treatment of genetic or acquired diseases include those encoding a deleterious gene product, such as an abnormal protein. Examples of non-limiting specific diseases include anemia, blood-related cancers, Parkinson's disease, and diabetes.

The present invention can be applied to develop autologous or allogeneic cell lines for therapeutical purposes. For example, gene therapy applications of particular interest in cell and/or organ transplantation are utilized with the present invention. In exemplary embodiments, downregulation of transplantation antigens (such as, for example, by downregulation of beta2-microglobulin expression via siRNA) allows for transplantation of allogeneic cells while minimizing the risk of rejection by the patient's immune system. The present invention would allow for a switch off of the RNAi in case of adverse effects (e.g. uncontrollable replication of the transplanted cells).

Cells types that can be subjected to the present invention include hematopoietic stem cells, myoblasts, hepatocytes, lymphocytes, airway epithelium, skin epithelium, islets, dopaminergic neurons, keratinocytes, and so forth. For further descriptions of cell types, genes and methods for gene therapy see e.g., Armentano et al. (1990); Wolff et al. (1990); Chowdhury et al. (1991); Ferry et al. (1991); Quantin et al (1992); Dai et al. (1992); van Beusechem et al. (1992); Rosenfeld et al. (1992); Kay et al. (1992); Cristiano et al (1993); Hwu et al (1993); and Herz and Gerard (1993).

In particular embodiments of the present invention, there is a method of treating any disease condition amenable to treatment with an s-ship promoter. In specific embodiments, the method comprises preparing a polynucleotide construct having a region encoding a therapeutic or diagnostic (marker) gene that is operably linked to an an s-ship promoter, wherein the gene encoded by the construct is for the treatment of the disease condition.

A. Pharmaceutical Formulations, Delivery, and Treatment Regimens

In an embodiment of the present invention, methods of treatment are contemplated. An effective amount of the pharmaceutical composition, generally, is defined as that amount sufficient to detectably and repeatedly to ameliorate, reduce, minimize or limit the extent of the disease or its symptoms. More rigorous definitions may apply, including elimination, eradication or cure of disease.

The routes of administration will vary, naturally, with the location and nature of the lesion, and include, e.g., intradermal, transdermal, parenteral, intravenous, intramuscular, intranasal, subcutaneous, percutaneous, intratracheal, intraperitoneal, intratumoral, perfusion, lavage, direct injection, and oral administration and formulation.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

B. Combination Treatments

The compounds and methods of the present invention may be used in the context of traditional therapies. In order to increase the effectiveness of a treatment with the compositions of the present invention, it may be desirable to combine these compositions with other agents effective in the treatment of those diseases and conditions. For example, the treatment of a cancer may be implemented with therapeutic compounds of the present invention and other anti-cancer therapies, such as anti-cancer agents or surgery. Likewise, the treatment of a vascular disease or condition may involve both the present invention and conventional vascular agents or therapies.

Various combinations may be employed; for example, a host cell of the present invention is “A” and the secondary anti-cancer agent/therapy is “B”:

| | | | |
|---|---|---|---|
| A/B/A B/A/B B/B/A | A/A/B A/B/B B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of that particular secondary therapy, taking into account the toxicity, if any, of the treatment. It is expected that the treatment cycles would be repeated as necessary. It also is contemplated that various standard therapies, as well as surgical intervention, may be applied in combination with the described therapy.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell Growth and Transfection Conditions

NIH3T3 cells, originally obtained from the American Type Culture Collection (ATCC, Rockville, Md.), were grown in DMEM with 10% fetal bovine serum. The D3 embryonic stem (ES) cell line was obtained from Dr. Tasulcu Honjo (Nakano et al., 1994) and grown in high glucose DMEM (GIBCO/Invitrogen Corp., #11965-092) supplemented with 2 mM L-glutamine, 1 mM sodium pyruvate, 0.1 mM nonessential amino acids, 0.15 mM monothioglycerol (Sigma, M7522), and 15% fetal bovine serum (pre-tested for ES cell growth (HyClone Labs, Inc.)). D3 ES cells were routinely grown on a LIF-producing feeder layer of mitomycin C-treated (Nagy et al., 2003) SNL cells, obtained from Phil Soriano (FHCRC). The SNL cells are G418-resistant. Usually, one passage before flow cytometry, ES cell were transferred to gelatin(Sigma)-coated plates without a feeder layer and with LIF (ESGRO) added to the medium (1000 units/ml).

DNA was transfected into D3 ES cells by electroportion essentially as described by Nagy et al, (2003). ES cells were suspended in PBS (Ca²⁺ and Mg²⁺-free) at 1×10⁶ cells/ml and 0.8 ml of the cell suspension placed in a 0.4-cm-wide electrode-gap sterile cuvette (BIO-RAD). Plasmid DNA (20 μg), linearized by overnight digestion with Afl II and Qiagen-purified, was added and mixed. Two pulses (instead of one as recommended) of current were applied to the cells in the cuvette employing settings of 500 mF, and 230V on a BIO-RAD Gene-Pulser™ with Capacitance Extender. After 5 min on ice, the viscous solution was transferred to a 10-cm culture dish containing mitomycin C-treated SNL cells. After 24 hr, G418 selection was begun using 280 μg/ml active G418. Cells were passed after 10-14 days onto gelatin-coated plates (no feeder cells) in LIF containing medium with G418. Flow cytometry was performed 3-4 days later.

Afl II-linearized plasmid DNA (10 μg) was introduced into NIH3T3 cells by transfection using Superfect reagent (Qiagen) as recommended by the manufacturer. G418 selection was begun 24 hr after transfection using 400 μg/ml G418. Cells were passaged twice in G418 before flow cytometry. Regardless of the electroporation into ES cells or transfection into the NIH3T3 cells, abundant G418 resistant colonies were obtained for each cell type.

Two positive control GFP-expression plasmids were used for both NIH3T3 cells and the D3 ES cells to be sure the transfection/electroporation steps were functional and that GFP expression occurred in each experiment. These positive controls also helped set the gates for analyses of GFP-expressing cells. These two plasmids were the pIRES2-GFP empty plasmid (BD Biosciences Clontech) and pIRES2-GFP containing an insert encoding the Capn5 gene. Both plasmids expressed equally well in each cell type, and the empty pIRES2-GFP vector always expressed higher levels of GFP than the one containing the insert.

Immunoblotting Analysis for SHIP Proteins

The techniques for cell extraction, electrophoresis, and immunoblotting have been described previously (Liu et al., 2001). Equal amounts of protein extracts from each cell type were loaded for gel electrophoresis. SHIP proteins were detected using monoclonal antibody P2C6 at a 1:1000 dilution (Lucas and Rohrschneider, 1999).

Flow Cytometry

Cells were examined for GFP expression on a Caliber II bench-top analyzer. Cytometer setting were established using positive FDC-P1 cells expressing GFP from a retroviral vector and negative cells, not transfected, or transfected with an empty plasmid. At least 10⁴ cells were analyzed for each plasmid transfected, and two independent transfections were examined. Both transfections gave similar results, and the results of one experiment are shown.

Construction of Promoter-Less GFP-Expression Constructs for Analysis of s-SHIP Intron-5 Promoter Activity

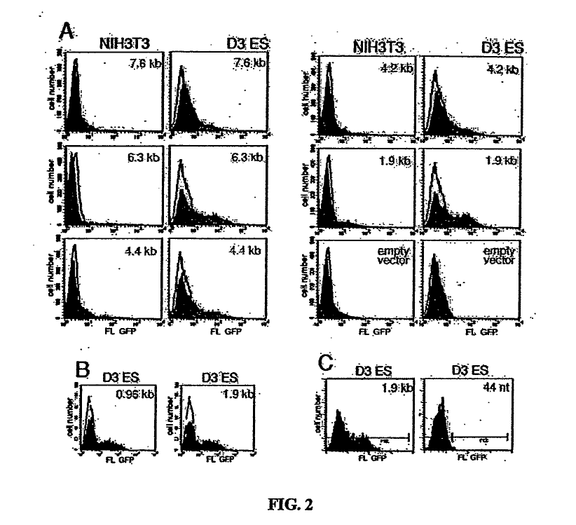

A 7.6-kb DNA Sac I-Sac I fragment from a Lambda 129Sv mouse genomic clone (Wolf et al., 2000, NCBI accession #AF235499, hereby incorporated by reference) was used for initial examination of potential tissue-specific promoter activity. This region contained almost all of intron-5, the 88 bp of exon-6, and 1271 bp extending into intron-6. This 7.6-kb segment was cloned into pBluescript KS (Stratagene), and sub-segments of the region were obtained with the restriction sites shown in FIG. 2. These sub-segments were cloned into a promoter-less GFP-expression construct.

The promoter-less GFP-expression construct was made from the pEGFP-1 plasmid (BD Biosciences Clontech) by modifications of the MCS (multiple cloning site), incorporating additional synthesized cloning sites (EcoRI-AccI(up)-BssHII-NheI-PstI) for insertion of the sub-fragments from the 7.6 kb intron-5 clone. Both AccI and BssHII recognize multiple sequences and the nucleotide sequence in the synthesized DNA corresponded to AccI site at nucleotide 2776 of the 7.6-kb region, and the 5′ BssHII site of the pBluescript plasmid, respectively. In addition, prior to incorporation of the extended MCS, the SV40 early and late introns from pCMVβ were inserted at the 3′ end of the MCS between the KpnI and AgeI sites. Two intron cassettes were used: one containing only the splice acceptor site from the long intron, and a second containing both early and late introns. The former was used only for inserts (e.g., the 7.6-kb and 4.2-kb inserts) containing an intact exon 6 with its splice donor site. The two final plasmids each containing the extended MCS and either the late SV40 intron only (pEGFP2-SD3-1), or both SV40 introns (pEGFP2-SD1-2), were sequenced through the inserted intron region and one of each with correct sequence selected for inserting the 7.6-kb clone and sub-regions.

The longest promoter construct contained the entire 7.6-kb putative s-SHIP promoter region, and was excised from the pBluescript plasmid with BssHII for insertion into the MCS of the pEGFP-SD3-1 plasmid. The 6.3-kb fragment was obtained with a partial PstI digestion and complete BssHII digestion. The 4.4-kb and 4.2-kb fragments were from derived from PstI and AccI digestions, respectively. The 1.9-kb segment was obtained from digestion of the 4.4 kb fragment with NheI. The smallest 0.96 kb region was produced by deleting a region of the pBluescript 7.6 kb clone from the SwaI site 960 nucleotides 5′ of exon 6, to the FbaI site 22 nucleotides from the 5′ end of the 7.6 kb clone. After ligation, the fragment from the 5′ BssHII site to the PstI site was excised. Each fragment was inserted into their respective restriction sites of the extended MCS. Restriction analysis of each purified plasmid confirmed the correct insert in the correct orientation, and all cloning junctions were sequenced to confirm proper ligation. Each plasmid was linearized with AflII, and Qiagen purified from agarose gels before electroporation or transfection.

Construction of the 11.5 kb- and 6.2 kb-GFP s-SHIP Promoter Transgenes

The 11.5 kb-GFP transgenic construct was prepared from two separate plasmids containing the two halves of the proposed s-SHIP promoter region, plus an 833 nt sequence from a lambda genomic clone, which was inserted between these two halves. The genomic organization of SHIP1 is shown in Wolf et al. (2000). The starting genomic clone contained a 4 kb region from the SacI site near the 3′ end of the 7.6 kb genomic clone in intron 6, extending through exon 8 and into intron 8. This SacI-SacI fragment was cloned into the SacI site of pBluescript SK (pBSK). The GFP gene from pEGFP-1 (Invitrogen/Clontech) was excised with NcoI (encompassing the ATG translation start site of GFP) and SspI. This was ligated into the NcoI (the putative s-SHIP translation start site in exon 7) and EcoRV sites of the pBSK-4 kb clone. Next, the 5′ half of the genomic promoter was added in the form of the SacI-SacI 7.6 kb genomic sub-clone. This was inserted into the one remaining SacI site at the 5′ end of the intron 6-exon 7-GFP clone in pBSK. This left a gap of 0.9 kb between the two SacI sites in intron 6 (see Wolf et al., 2000). This region was recovered as a larger BsiWI-EcoRI 2117 nt fragment, whose sequence demonstrated the insertion of 833 nucleotides between two SacI sites. Therefore, this BsiWI-EcoRI fragment was inserted into the same unique sites of the transgenic construct to produce the finished 11.5 kb-GFP transgene in pBSK.

The 6.2 kb-GFP transgene-construct was prepared from the 11.5 kb-GFP transgene prior to the insertion of the 833 nt at the intron 6 SacI site. This 11.5 kb(Δ833)-GFP construct was digested with FbaI and Swal, removing 5.3 kb from the 5′ end of intron 5. Re-ligation removed all but 19 intron 5 nt at the 5′ end of the 11.5 kb-GFP transgene. Both 11.5 kb-GFP and 6.2 kb-GFP transgenes, in pBSK, were cut from the plasmid with BssHII and Qiagen purified from an agarose gel for introduction into the mouse genome.

Production of Transgenic Mice

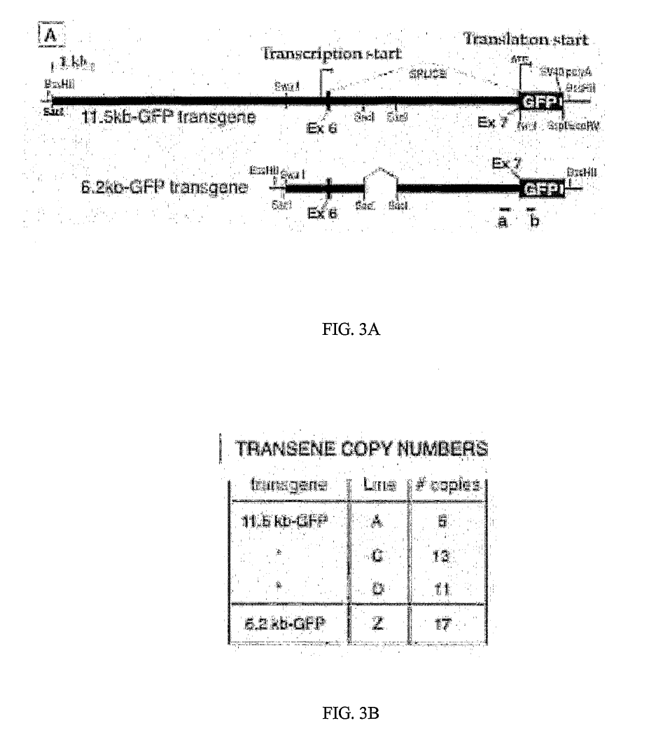

Founder transgenic mice were prepared in our Transgenic Mouse Facility by pronuclear injection of fertilized zygotes from (C57B1/6 female X CBA/J male) F1 mice. Mice, positive for the transgene, were screened by PCR using DNA obtained from tails or toes of young animals. The location of the primer set for PCR is shown in FIG. 3: the upstream primer (a) is within intron 6 (Pro-up-2,5′-TACTCCTCAGCAAGAGTAGCTGG-3′) (SEQ ID NO:12), and the downstream primer (b) within the GFP gene (GFP-dnl, 5′-GCTGAACTTGTGGCCGTTTACGT-3′) (SEQ ID NO:13) produce a 632 nucleotide (nt) product. These primers were used for detection of both 6.2 kb-GFP and 11.5 kb-GFP transgenic mice. Positive chimeric mice were bred to C57B1/6 mice and four founder lines (A, B, C and D) obtained for the 11.5 kb-GFP mice. Later analyses demonstrated that founder line B was not positive for GFP expression, even though the primer pair a and b gave a positive 632 nt product. Therefore, line B is not included in further analyses. The other lines were maintained by breeding transgene-positive animals with wild-type C57B1/6 mice. For some experiments transgene-positive offspring were generated from positive intra-line breeding. Two founder animals were obtained for the 6.2 kb-GFP transgene but one was lost.

The transgene copy number in each founder line (except 11.5 kb-GFP, line B) was determined by semi-quantitative RT-PCR of transgene expression relative to endogenous Gab2 expression. Primers for detecting genomic gab2 are: E4F, 5′-CTTCTATAGCCTTCCCAAGCC-3′ (SEQ ID NO:14); E5R, 5′-CTCGTAGGTCTCACAGGAAG-3′ (SEQ ID NO:15).

Analysis of Embryos

Preimplantation embryos were harvested at 2.5 and 3.5 dpc from uterine horns of pregnant females [see Nagy et al, (2003) for details of these methods]. The morulae and blastocysts were washed in RPMI 1640 medium (Gibco) containing 10% fetal bovine serum, transferred to PBS (Ca²⁺ and Mg²⁺), and GFP-expression or phase images photographed on a Nikon Eclipse TE200 inverted microscope coupled to a Roper Scientific 1 k×1 k pixel digital camera. Images were captured with MetaMorph software and prepared for publication with Photoshop (Adobe). High-resolution z-sections of GFP expression within embryos were made with a Leica TCS SP Confocal microscope.

Several blastocysts were plated onto gelatin-coated tissue-culture wells in DME 10% fetal bovine serum, and photographed three days later. During this period, blastocysts hatched from the zona pellucida, and attached to the culture plate. The attached mass of trophectoderm cells with the non-adherent ICM was photographed for GFP and phase with a Nikon Eclipse TE200 microscope.

RT-PCR Analysis of s-SHIP Expression in Blastocysts

mRNA was isolated from wild-type 3.5 dpc blastocysts, FDC-P1 cells and the D3 ES cells using a Dynabeads mRNA DIRECT micro kit (Dynal). Reverse transcription used the Sensiscript kit from Qiagen, and the PCR cycling conditions were as follows: 94° C. 1 min, [94° C. 15 sec, 68° C. 2 min]×30 cycles, 68° C. 5 min, and a 4° C. hold. Each reaction used the equivalent of 1.5 ng mRNA, based on the concentration before reverse transcription. Primers pairs were:

| | |
|---|---|
| HPRT-up1, | |
| 5′-CCTGCTGGATTACATTAAAGCACTG-3′, | (SEQ ID NO:16) |
| HPRT-down1 | |
| 5′-GTCAAGGGCATATCCAACAACAAAC-3′; | (SEQ ID NO:17) |
| OCT4-Up1 | |
| 5′-GGCGTTCTCTTTGGAAAGGTGTTC-3′, | (SEQ ID NO:18) |
| OCT4-Down1 | |
| 5′-CTCGAACCACATCCTTCTCT-3′; | (SEQ ID NO:19) |
| SHIP1/s-SHIP pair #3, | |
| SHIP-E8FW, | |
| 5′-TTGCTGCACGAGGGCTCAGAATC-3′, | (SEQ ID NO:20) |
| SSP883RV, | |
| 5′-TCCGATTCTCATGCTCTGGCTTG-3′; | (SEQ ID NO:21) |
| SHIP1/s-SHIP pair #4, | |
| SP2109FW, | |
| 5′-CAGCCCTGTCTTTGCCACGTTTG-3′, | (SEQ ID NO:22) |
| SP2637RV, | |
| 5′-TCCACTGGATTCATCCCGCTCTG-3′; | (SEQ ID NO:23) |
| SHIP1/s-SHIP pair #5, | |
| newfw, | |
| 5′-CTTCCTCTTGCAACAGAGAACCC-3′, | (SEQ ID NO:24) |
| newrv, | |
| 5′-ACTCAACGTCCACTTTGAGATGC-3′. | (SEQ ID NO:25) |

### Example 2

### Identification and Characterization of the s-SHIP Promoter

Potential s-SHIP promoter activity was first analyzed in cell lines grown in culture. Several cell lines were tested for s-SHIP vs. SHIP1 protein expression, based on the known and expected expression pattern of the s-SHIP protein (Lioubin et al., 1994; Tu et al., 2001). These results showed the expression of the ˜104-kDa s-SHIP only in the ES cells, whereas the 145-kDa SHIP1 product was exclusively expressed in the maturing FD-Fms myeloid cells. Hot SDS-extraction of the ES cells did not change the size of the s-SHIP protein, suggesting that this 104-kDa product is not the result of proteolytic degradation during extraction (Horn et al., 2001). SHIP proteins were not detectable in NIH3T3 fibroblasts, the SNL cells serving as feeder for the ES cell growth, or the 293 human kidney cells. Therefore, NIH3T3 cells and D3 ES cells were selected as negative and positive cells, respectively, for analysis of the potential s-SHIP promoter activity.

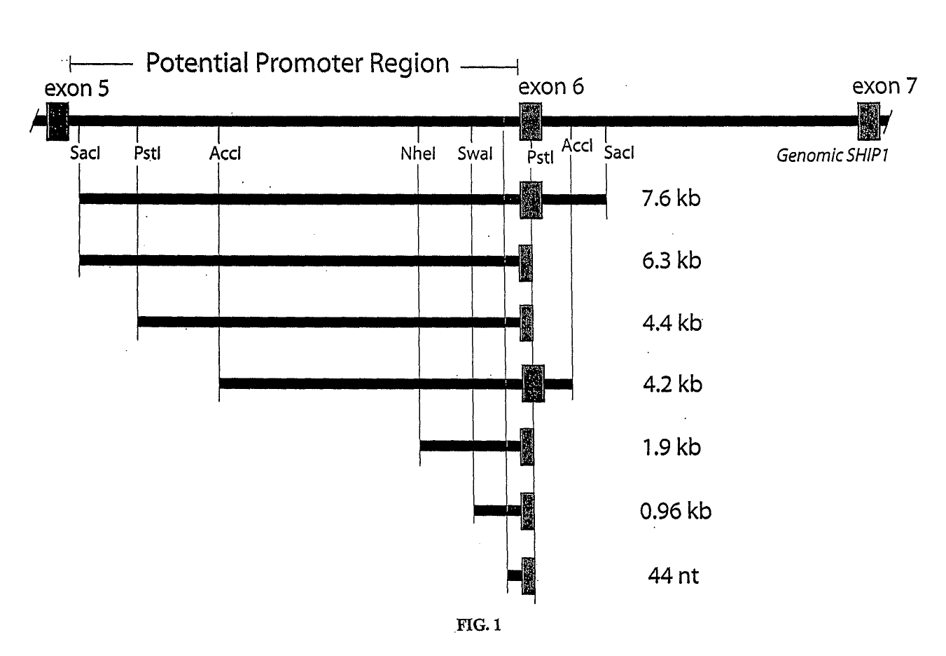

A 7.6-kb genomic ship1 region containing the intron-5 region was obtained for initial promoter analysis. The entire 7.6-kb region and sub-fragments thereof were cloned into a promoter-less GFP (enhanced green-fluorescent protein) expression vector (FIG. 1). Promoter activity of the intron-5 region was then assayed in the cells positive for s-SHIP expression (embryonic stem cells, clone D3) vs. cells negative for s-SHIP expression (NIH3T3 cells). The expression of GFP in each cell type, assayed by flow cytometry, was a measure of the promoter activity within each fragment of the 7.6 kb genomic DNA. The results indicated that, whereas, empty vectors alone lacked significant promoter activity in either cell type, vectors containing intron-5 segments exhibited substantial expression in the D3 ES cells but not in the NIH3T3 cells. Segments of intron 5, ranging from 0.96 kb to 7.6 kb were active for GFP expression in the ES cells; however, the shorter segments appeared most active. Two fragments of 1.9 kb and 0.96 kb, immediately upstream of exon 6, each exhibited equally high GFP expression. The shortest insert fragment contained part of exon 6, but only the 44 nucleotides upstream of exon 6, (Tu et al., 2001), and was completely without promoter activity. These results strongly suggest that the intron-5 region of genomic ship1 contains cell-specific promoter activity, and segments more distal to exon 6 may have negative regulatory activity.

Based on the ES/NIH3T3 cell-transfection experiments, two new constructs with an extended region downstream of the intron-5 genomic area were prepared for in vivo analysis of promoter activity in transgenic mice (FIG. 3A). Transgenic mice were produced for in vivo examination of the putative s-SHIP promoter/enhancer activity, and determining the overall expression pattern of the transgene, and presumably s-SHIP protein. The promoter in the longer of the new constructs (the 11.5 kb-GFP transgene) contained the entire intron 5 from the above 7.6-kb genomic fragment, plus all of exon 6, intron 6, and the portion of exon 7 ending at the theoretical ATG start site (Kozak, 1987) for the s-SHIP protein translation. This start site was fused, in frame, to the ATG for the GFP protein. All of intron 6 and part of exon 7 were included in this construct because, 1) the construct might then more closely resemble the endogenous promoter, 2) splicing may be important for efficient expression (Nott et al., 2004), and 3) positive or negative regulatory elements for expression may also reside within this sequence. The second, shorter, transgenic promoter construct (the 6.2 kb-GFP transgene) was similar, but contained only 0.96 kb of intron 5 sequence adjacent to exon 6, and also lacked 833 nucleotides between two SacI sites within intron 6. Thus, if either construct contained promoter activity in vivo, transcription would start within intron 5, while intron 6 would be spliced out and translation of GFP would begin at the first ATG within an appropriate Kozak site.

Transgenic (Tg) mice were then produced in the Hutchinson Center Transgenic Mouse facility and chimera animals screened for each transgene by PCR. Breeding each founder to wild-type C57B1/6 mice yielded four lines containing the 11.5 kb-GFP transgene, and one line with the 6.2 kb-GFP transgene. Of the four founder Tg11.5 kb-GFP mice, one was negative for expression of the transgene (line B), while three were positive and each has exhibited the same expression patterns (lines A, C and D). Copy numbers of genomic transgenes, measured relative to the endogenous gab2 gene are shown in FIG. 3B. Within the three GFP-expressing 11.5 kb-GFP founder mice, empirical results indicate that line C exhibits the noticeably highest GFP expression levels. Line C mice also exhibit lower birth rates with in utero death at 8.5-9.5 days postcoitum (dpc) apparent. The single 6.2 kb-GFP founder line harbors the most transgene copies, but no overt defects in the physical appearance of these mice, their birth rate or development have been observed.

Experiments were then conducted with the adult transgenic 11.5 kb-GFP mice to examine transgene expression; however, it was difficult initially to find any GFP expressed in these mice by flow cytometry of blood and stem cell enriched bone marrow. After several negative attempts to find GFP expression, it was reasoned that because ES cell expression was readily detectable in the initial ES cell experiments, the best test for in vivo expression would be the inner cell mass (ICM) of the blastocyst, from which ES cells can be derived. Therefore, we looked for GFP expression in 3.5-dpc blastocysts derived from mating of Tg males×WT females. Blastocysts derived from one such cross produced 9 GFP-positive embryos indicating that the Tg was homozygous for the transgene. A separate Tg male bred to a WT female produced both positive and negative blastocysts. GFP-positive morulae were also obtained from similar crosses; whereas, blastocysts or morulae from WT parents were negative for GFP.

Blastocysts are composed of 2-3 cell types depending on their developmental stage. The outer trophectoderm layer of cells surrounds the eccentric inner cell mass (ICM), destined to become the embryo proper, and later stage blastocysts also contain endodermal cells separating the ICM from the blastocoel cavity (Nagy et al., 2003). To obtain a better idea of which cells of the blastocyst express the GFP transgene, transgenic 3.5-dpc blastocysts were allowed to adhere to a culture dish by three days growth in DME 10% FBS. Under these conditions, the zona pellucida is shed, and the outer trophectoderm cells of the blastocyst form an adherent layer while the ICM remains as an unspread mass, and each is distinguishable morphologically from the other. The results showed that the ICM portion of the blastocyst retained the GFP expression while the adherent trophectoderm cells were largely GFP-negative.

A more detailed picture of GFP expression throughout the intact early pre-implantation embryos was seen in confocal Z-sections of GFP within transgenic 2.5-dpc morulae and 3.5-dpc blastocysts. All cells of the 16 to 32-cell morula were GFP-positive. Transition of the morula to the early blastocyst is marked by the formation of the blastocoel cavity. A few cells of this early blastocyst structure began to shut-off GFP expression, and the extent of this GFP shut-off was more evident in the late blastocyst. Here, the outer trophectoderm cells had noticeably lower GFP expression, and the GFP-positive cells were confined to the ICM. Endodermal cells were not readily apparent. In these images, it is helpful to remember that the half-life of the GFP fluorescence is greater than 24 hr (Tech. Borchure, BD Bioscience ClonTech), and therefore cells, which have stopped expressing GFP, will retain some GFP protein and fluorescence for several days. Twenty-four hours separates the morula from the blastocyst stages; therefore, transgene shut-off early during this time would result in lower but not complete lack of GFP fluorescence late in this time span. The 11.5-kb transgene s-SHIP promoter contains the information for both cell-specific positive expression in morula and ICM of the blastocyst, but also cell-specific shut-off in trophectoderm cells.

Preimplantation embryos from the Tg6.2 kb-GFP mice were analyzed next. The transgene in these mice contained only the proximal 0.96-kb region upstream of exon 6, which was necessary for GFP expression in the ES cells. It also lacked 833 nucleotides between two SacI sites of the intron-6 region. GFP expression in the 3.5-dpc blastocyst of the 6.2 kb-GFP line was analyzed. Both qualitative and quantitative features of GFP expression in the Tg6.2 kb-GFP blastocysts differed from those in the Tg11.5 kb-GFP mice. First, GFP expression in the Tg6.2 kb-GFP blastocysts was noticeably stronger (at least 5-fold) than that in the Tg11.5 kb-GFP blastocysts, as measured by exposure times for obtaining equivalent GFP images in the Nikon digital microscope. Second, and more noticeable was the lack of GFP shut-off in the trophectoderm cells of the blastocyst. No clear demarcation in GFP expression was evident between ICM vs. trophectoderm as seen in the Tg11.5 kb-GFP blastocysts.

Blastocysts from the Tg6.2 kb-GFP mice were also allowed to adhere to culture plates and GFP expression was examined. Adherent blastocysts from Tg11.5 kb-GFP mice were examined simultaneously. Adherent Tg6.2 kb-GFP blastocyste expressed GFP in both ICM and trophectoderm cells in a, frequently, haphazard pattern. The Tg11.5 kb-GFP adherent blastocysts expressed GFP only in the ICM as observed previously. A comparison of all embryos examined revealed that an increased GFP expression was apparent within the adherent Tg6.2 kb-GFP blastocysts relative to the adherent Tg11.5 kb-GFP blastocysts. These results were consistent with the promoter analyses performed in the ES cells (FIG. 1), and suggested that the lack of GFP shut-off by the 6.2 kb-GFP transgene was due to negative regulatory information found in either one or both regions of the 11.5 kb-GFP construct missing from the 6.2 kb-GFP transgene.

The data from Tu et al (2001) and that presented herein demonstrated exclusive s-SHIP (rather than SHIP1) expression in ES cells, yet, even though ES cells are derived from the ICM of the blastocyst and the intron 5 s-SHIP promoter functioned well in the ICM, it was still not certain whether the ICM actually expressed s-SHIP in vivo. Consequently, s-SHIP mRNA expression was then analyzed by RT-PCR, compared to that of the universally expressed HPRT, and the ES cell and ICM-specific Oct4 transcription factor. RNA from blastocysts, FDC-P1 myeloid progenitor cells, and D3 ES cells, was positive for HPRT as expected, and only the blastocysts and ES cells were positive for Oct4. Initially, the s-SHIP-specific primers similar to those described by Tu et al. (2001) was used to test for s-SHIP expression; however, poor results were obtained. The forward primer in this set was moved 3′-ward into the region identical to SHIP1 but weak detection was still obtained. s-SHIP was therefore detected by “subtraction” using primers detecting both s-SHIP and SHIP1 products vs. primers detecting only the SHIP1 product. These primers clearly demonstrated the presence of full-length SHIP1 only in the FDC-P1 cells, and s-SHIP in both blastocysts and ES cells. The weak detectability of s-SHIP may be due to poor hybridization of the primers, degradation of the 5's-SHIP mRNA ends, or possibly an additional shorter transcription product from the ship1 gene.

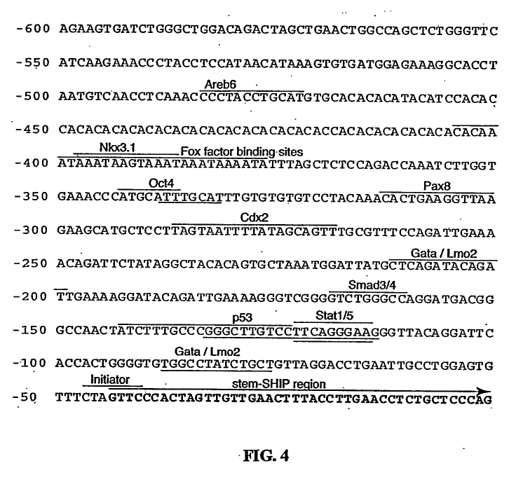

Examination of the minimal 0.96-kb promoter proximal to exon 6 by MatInspector indicated at several transcription-factor binding site potentially active in ES cells and the blastocyst ICM. FIG. 4 shows the first 600 nucleotides of this region upstream of exon 6, with potential transcription factor binding sites and motifs for transcriptional regulation marked. A transcription initiator sequence (Butler and Kadonaga, 2002) straddles the 5′ end of the 44 nt SSR, suggesting a transcriptional start site. Paired GATA, or Lmo2 binding sites are present, two overlapping p53 and Oct-binding sites, and a single extended FOX-factor binding region are prominent motifs. The Oct-binding motif is present in similar regions of both the murine and human s-SHIP promoter, suggesting such a factor could be important for ES and ICM expression. The POU factor Oct4 is expressed in ES cells and is part of an enhancer for ES cell-specific expression of target genes (Dailey et al., 1994). Therefore the Oct site could be part of a similar ES cell enhancer region.

The transgene expression in preimplantation embryos raises a question about possible progenitor transgene expression in the oocytes or sperm of the adult, which then give rise to the fertilized embryo. The transcription factor, Oct4, is expressed in adult and embryonic germ cells, as well as the blastocyst ICM and in ES cells (Pesce et al., 1998). The possibility that the 11.5 kb-GFP transgene could also be germ cell specific is even more likely given the prominent Oct4 binding motif within the 0.96 kb minimal promoter upstream of exon 6 (see FIG. 4). Therefore, ovaries and testes from 7-8 week old adult Tg11.5 kb-GFP mice were harvested and frozen sections stained with Alexa 594-labeled phalloidin for visualizing tissue structure through polymerized actin staining, and endogenous GFP expression. The results of this experiment demonstrated that neither the developing sperm of the testis, nor the developing oocytes of the ovarian follicles expressed GFP. Only blood vessels of the testes and ovaries exhibited specific GFP expression. Therefore, unlike the Oct4 transcription factor, the 11.5 kb-GFP transgene is not a maternally activated gene, must be transcriptionally activated sometime after the germ cells leave the ovary/testis, and before the 2.5-dpc-morula stage of development.

### Example 3

### Further Characterization of the s-SHIP Promoter

The transgenic mice that were generated from the experiments described in Example 2 were further analyzed by immunofluorescence. Embryos were harvested, washed, and fixed 2-4 hr in 2% paraformaldehyde in PBS, then washed in 30% sucrose in PBS and stored in that solution overnight at 4° C. Embryos were frozen in O.C.T. on dry ice and stored at −80° C. until sectioned. Twenty mm sections on Superfrost/Plus (Fisherbrand) microscope glass slides were air-dried overnight and stored, desiccated, at −20 C. Sections were routinely stained with a rabbit anti-GFP antibody coupled to Alexa 488 (Molecular Probes) to enhance the transgenic GFP detection. For general screening, sections were also stained with phalloidin coupled to Alexa 594 (molecular Probes) for detecting morphology by filamentous actin staining. Other antibodies used were specific for: CD45-Cy-Chrome labeled, and Flk1 (VEGFR2) phycoerythrin labeled (both from Pharmingen); Oct4, mouse monoclonal (Santa Cruz Biotechnology); E-cadherin, rat monoclonal (Zymed); and alpha smooth muscle actin, mouse monoclonal (Sigma). The BM alkaline phosphatase detection reagent was from Roche. The tissue sections were blocked in 5% fetal bovine serum for 30 min. washed in PBS, treated 10 min in 0.5% TX-100 in PBS then washed again in PBS. The primary antibodies were applied and sections incubated at RT in a humidified chamber for 45-60 min. Sections were washed 3-times 10 min each in PBS and secondary antibodies added and incubated as before. Final washing was 3-times 15 min and sections were mounted in ProLong (Molecular Probes). All secondary antibodies were from Molecular probes and labeled with Alexa 594 or Alexa 633. Slides were viewed with a Leitz TCS SP Confocal microscope.

s-SHIP expression was tested in blastocysts by RT-PCR, compared to that of the universally expressed HPRT, and the ES cell and ICM-specific Oct4 transcription factor (Pesce et al., 1998). FDC-P1 myeloid progenitor cells express SHIP1 but not s-SHIP (Lucas and Rohrschneider, 1999; Tu et al. 2001), while conversely, D3 ES cells express only s-SHIP (Tu et al. 2001; data not shown). These two cell types represented the positive and negative controls. RNA from FDC-P1, D3 ES cells and 3.5 dpc blastocysts were tested and each was positive for HPRT, while mRNA from the blastocysts and ES cells, but not from the FDC-P1 cells, was positive for Oct4, as expected. s-SHIP was detected by “subtraction” using primers common to both s-SHIP and SHIP1 vs. primers detecting only the SHIP1 product. These primers demonstrate the presence of full-length SHIP1 only in the FDC-P1 cells, and s-SHIP in both blastocysts and ES cells. Analysis of transgene expression in the post-implantation mouse embryo.

Ongoing development of the blastocyst following implantation of the embryo continues with the formation of the epiblast (the embryo body) from cells comprising the blastocyst ICM. Consistent with this derivation, the epiblast in the Tg11.5 kb-GFP E6 embryo retained GFP expression; however, within 24 hr, epiblast GFP expression was lost. E7-7.5 embryos exhibited individual GFP-positive cells, or groups of cells, in the extraembryonic membranes, often appearing to trail from the epiblast. By 8.5 dpc, GFP expression could no longer be seen in the embryo body itself, but both the yolk sac and placenta contained numerous GFP-positive cells.

Serial sections through the transgenic E8.5 decidua and growing embryo have not detected specific GFP expression in any tissue or cells of the embryo body. Specifically, GFP-positive migrating primordial germ cells (PGCs) have not been detected in the hindgut region of the E8.5 embryos (not shown, but see later results). Within the extraembryonic regions, however, the ectoplacental plate (also called chorioallantoic plate) contained patches of GFP-positive cells, and both the blood islands and endoderm cell layer of the yolk sac contained individual or small groups of GFP-positive cells. Occasional intense GFP⁺ round cells within the primitive erythrocyte-filled blood islands were observed, and groups of GFP⁺ cells adjacent to blood islands were also seen. GFP⁺ cells of the peripheral ectoplacental plate sometimes appeared contiguous with GFP⁺ endodermal cells of the yolk sac. Maternal placental contributions did not account for the yolk sac nor ectoplacental plate expression profiles, because the same GFP expression patterns were observed in embryos from Tg males mated to WT females (not shown). This GFP expression pattern in the yolk sac is similar to that reported using an enhancer derived from the Scl(Tal1) stem cell protein (Sanchez et al., 1999), suggesting that these GFP⁺ cells may be related to hemangioblasts. However, Scl(Tal1) expression was not detected in the E8.5 GFP⁺ extraembryonic membrane cells.

In contrast to the lack of GFP expression within the 8.5-dpc embryo proper, whole-mount observations of 11.5-dpc Tg11.5 kb-GFP embryo showed dramatic GFP expression in the caudal region of the embryos and a distinct pattern of GFP expression on, and around, each hindlimb/forelimb pair. This complex pattern represented multiple distinct expression sites. At this age of development, the strongest and most broadly observed site of GFP expression was the epidermal cell layer of the developing skin. This was seen in whole mounts but demonstrated best in frozen sections of E11.5 transgenic embryos. The E11.5 transgenic embryos exhibited extensive GFP expression in the body and limbs, but such expression was not seen in the head region at this embryonic stage. A second epidermis-related GFP expression site was the apical ectodermal ridge (AER) of each developing limb, and a third distinct pattern was the mammary buds. These latter structures form by invaginations of the skin epidermis and were observed as five GFP-positive spots between each forelimb/hindlimb pair, corresponding to the bilateral three thoracic and two inguinal developing mammary glands. These were seen in whole mount embryos or underneath the dissected epidermis.

An additional prominent GFP expression site in 11.5-dpc Tg11.5 kb-GFP embryos was the genital ridge where PGCs were accumulating. The dissected aorta-gonad-mesonephros (AGM) region from an E13.5 embryo demonstrated the localization of the GFP⁺ cells within the gonads. The PGCs co-express the nuclear transcription factor Oct4 within the GFP⁺ cells, identifying these GFP⁺ cells as PGCs. GFP expression was not detected above background in the liver and doral aorta. The dorsal aorta is also present in the dissected AGM between the two gonads, but specific GFP expression was not observable at this site. The absence of GFP expression in the dorsal aorta suggests a lack of any relationship to definitive hematopoiesis, reportedly arising from this region (Dzierzak, Medvinsky, and de Bruijn, 1998).

Primordial Germ Cells

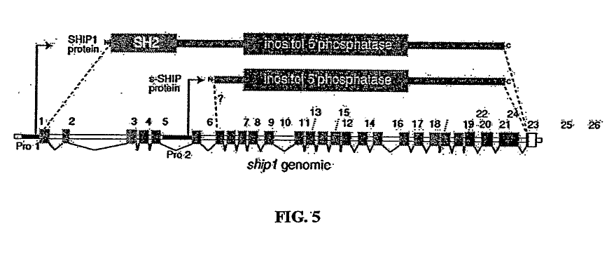

GFP expression in the PGCs of the Tg11.5 kb-GFP embryo was examined in more detail during E9.5-E18.5 stages of embryonic development. Using Oct4 as a marker for PGCs, the temporal co-expression of GFP and Oct4 was followed from embryonic day 9.5 to 18.5. Consistent with earlier results, GFP was not expressed in the earliest E9.5 PGCs migrating along the hindgut, although Oct4 was present in their nuclei (FIG. 5Aa,b,c). At E11.5, PGCs of the genital ridges contained GFP and nuclear Oct4. Both E13.5 and E17.5 PGCs were GFP⁺ and Oct4⁺. Together with the earlier analyses of the E8.5 embryos, these results suggest that GFP is not expressed in the early migrating PGCs of the E8.5-9.5 embryo, but is readily detected in the PGCs of the genital ridge and gonads of the E11.5-17.5 embryos.

GFP expression was observed in PGCs from an E13.5 embryo ovary. Many of the GFP⁺ PGCs cells at this stage were in cell division. Primitive seminiferous tubules are distinguishable in the E15.5 testes, populated with GFP⁺ PGCs and developing spermatogenic cells (Kaufman, 2001). The brightest GFP⁺ cells at this stage were in mitosis and these may represent the type A spermatogonia undergoing cell division (Kaufman, 2001). The seminiferous tubules of the E18.5 embryo were, likewise, filled with GFP spermatogenic cells. Sertoli cells attached to the basement membrane of the seminiferous tubule were GFP-negative. Germ cells in both ovaries and testes were positive for GFP in the E13.5-E18.5 stages of embryo development. However, quite surprisingly, neither ovaries nor testes of adults expressed GFP in any stage of germ cell formation. These observations indicate that the expression of the 11.5 kb-GFP transgene is positively regulated during the E11.5-18.5 developmental stages, but negatively regulated in the adult.

11.5 kb-GFP Transgene Expression in Other Tissues from E15.5-E18.5 Embryos

Following E11.5 in the Tgl1.5 kb-GFP embryo, new GFP⁺ structures are observed. Specifically, the developing cornea of the 15.5-dpc embryo eye was GFP⁺. The cornea is formed from an outer epithelial layer and an inner cell layer derived from neural crest cells. Only the outer epithelial layer showed GFP expression⁺. The retina, derived from neuroepithelium, did not express GFP, while the lens, although an invagination of the epidermis, was likewise GFP-negative.

E15.5 embryos began to exhibit GFP expression in cells surrounding blood vessels, and this expression became more noticeable at E18.5. The expression was restricted to smaller vessels and not expressed in larger arteries (e.g., aorta) or veins. The vessel-associated GFP was expressed in cells wrapping around the circumference of the vessels. This characteristic suggests the GFP⁺ cells are smooth muscle cells, and this notion was supported by the observation that alpha-smooth muscle actin co-localized with the GFP vessel cells. GFP was not detectable in all vascular smooth muscle cells (vSMCs), and was also not detected in cardiac or skeletal muscle smooth muscle cells. This indicates a highly specific regulation of the 11.5 kb-GFP transgene only in a population of the smooth muscle cells associated with small blood vessels.

A few scattered cells in the E15.5 thymus were also positive for GFP. Morphologically, these cells did not appear to be blood derived, but rather adhered to the thymus stroma via E-cadherin, suggesting an epithelial nature.

The paired vomeronasal (Jacobson's) organ in the nasal septum also expressed GFP. Jacobson's organ is derived from the neuroepithelium, again suggesting a potential connection between transgene expression and epidermal-derived tissues.

The E17.5-18.5 transgenic embryos exhibited GFP expression in cells associated with the forming bone matrix. These small irregularly shaped GFP⁺ cells are attached to the newly formed bone and are most likely the osteoblasts in the process of converting extracellular matrix material to bone (Komori, et al., 1997; Long, et al., 2004). This notion was confirmed by demonstrating the colocalization of alkaline phosphatase and GFP in these cells. Trabecular bone also exhibited alkaline phosphatase-positive/GFP⁺ osteoblasts.

Transgene Expression in Embryonic Epidermis, Tissues Derived from Epidermis, and Other Epithelial Cells

Embryonic skin is a major stem/progenitor cell population essential for formation of several appendages and tissues, such as, hair follicles, sweat glands, mammary tissue, and (more indirectly) prostate. Therefore, these epidermal-derived tissues were examined for GFP expression in Tg11.5 kb-GFP embryonic mice.

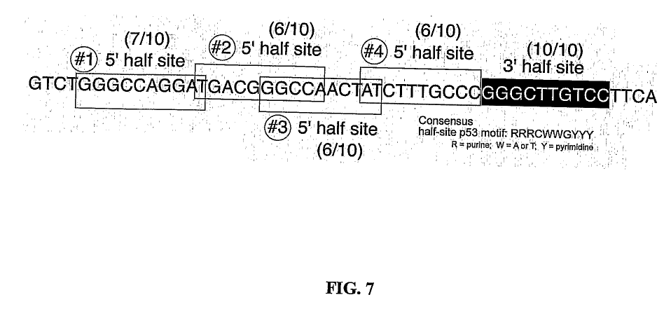

Hair follicle formation initiates around E13.5 by reciprocal inductive signals between skin epidermis and underlying mesenchyme (Miller, 2002). The resultant hair follicle placodes exhibit a localized epidermal thickening in a geometrically ordered array due to inhibitory signaling from each placode (Andl et al., 2002). The results demonstrated that E13.5 transgenic embryos exhibit a geometric array of GFP⁺ speckles in the skin, suggesting GFP expression in the hair follicle placodes. Frozen sections of skin at this stage revealed GFP⁺ epidermal thickening indicating placode formation. Growth and extension of the placodes into the mesenchyme produces the hair follicle sheath with a distal bulb (Alanso and Fuchs, 2003). Early stages of hair follicle formation showed the incorporation of GFP⁺ skin epidermal cells into the follicle extending about one cell diameter into the mesenchyme. The dermal component of the follicle (the dermal papilla) is GFP⁻, and clearly visible in FIG. 7C adjacent to the GFP⁺ cells. As the follicle extends further, the GFP⁺ cells remain as a small cluster, perhaps maintaining their epidermal-cell niche. During further growth of the hair follicle, the GFP⁺ cell cluster remained intact at the distal bulb end of the growing follicle, and the follicle retained the E-cadherin expression, as did the epidermis from which it was derived. Frequently, the base of each growing hair follicle (anchored in the epidermis) lacked significant GFP expression, and the GFP⁺ cluster resides at the distal bulb end of the follicle.

Mammary bud formation also occurs from skin epidermis by epidermis-mesenchyme induction, but differs in the time of initial formation, the size of placodes, and morphology of the developing tissue. The mammary buds were visible in whole mount immunofluorescence of the E11.5 transgenic embryos, and frozen sections taken at the same stage show GFP+mammary placodes. These placodes became round-up and extended into the mesenchyme forming the buds, which subsequently formed larger bulbous structures as they grew. The results also demonstrated the size of a hair follicle relative to a mammary bud at this stage. This growing embryonic mammary tissue exhibited a few GFP⁺ cells at the outside periphery of the tissue. The expanding mammary tissue remained attached to the epidermis and the nipple formed around this attachment site.

A third tissue derived from epithelium is the prostate organ. However, unlike hair follicles and mammary buds, prostate is formed from urothelium by poorly understood signals at the base of the bladder in the E17.5 male mouse. Frozen sections of the prostatic region of the E18.5 Tg11.5 kb-GFP male mouse demonstrated specific GFP expression in regions destined to become lateral lobes of the prostate. Serial sections through this region showed GFP expression only in the prostatic region, and non-transgenic mice lacked this expression. Overall, these results indicated that epidermal/epithelial tissue constitutes a major target for expression of the 11.5 kb-GFP transgene, and tissues derived from epithelium appear to obtain (and maintain) stem cells from these sources.

Expression of GFP from the Shorter 6.2 kb-GFP Transgene in the Mouse Embryo

The 6.2 kb-GFP transgene mice were produced for the purpose of obtaining information about expression capability of promoter segments compared to the 11.5 kb-GFP transgene mice. In pre-implantation embryos the shorter transgene was strongly expressed throughout the blastocyst and no delineation was apparent between the ICM and trophectoderm cells in the intact blastocyst, or in blastocysts allowed to adhere to tissue culture plastic. This lack of ICM specificity could be due to the higher copy number in the single Tg6.2 kb-GFP founder, and/or to the higher expression of the transgene. Regardless, later developmental stages exhibited highly tissue-specific expression patterns, and dramatic differences in the 6.2 kb-GFP transgene expression compared to the 11.5 kb-GFP transgene were observed. The qualitative differences were not yet apparent in E8.5 embryos; however, at this stage the Tg 6.2 kb-GFP E8.5 embryos no longer expressed GFP ubiquitously but only cells of the extraembryonic membranes were GFP⁺. This expression was similar to that observed with the same stage Tg11.5 kb-GFP embryos; however, yolk sac expression was not observed in the Tg6.2 kb-GFP mice. Surprisingly, unlike the Tg11.5 kb-GFP mice, the E11.5-13.5 Tg6.2 kb-GFP embryos were devoid of significant GFP expression. Thus, at these developmental stages two of the most prominent GFP expression sites in the Tg11.5 kbFP embryos (i.e., skin epidermis and PGCs in the genital ridge and gonads) were completely absent in the Tg6.2 kb-GFP embryos.

At still later developmental times, E18.5, two of the GFP expression sites seen in the Tg11.5 kb-GFP embryos were observed in the Tg6.2 kb-GFP embryos. These sites were the blood vessels and the osteoblast cells attached to the forming bone matrix. GFP was expressed in the smaller blood vessels, but not all small vessels expressed GFP.

Therefore, the expression of the 6.2 kb-GFP transgene within the embryo was limited to fewer tissues than observed with the Tg11.5 kb-GFP mice, and represented a subset of expression sites seen within the Tg11.5 kb-GFP mice at the same developmental stage. These results suggest that the promoter sequence remaining within the 6.2 kb-GFP transgene contains the information (enhancers) for tissue-specific expression of GFP in cells of the extraembryonic membranes at E8.5 and in the smooth muscle cells surrounding blood vessels, and osteoblast in late stage embryos. Conversely, the genetic sequences present in the 11.5 kb-GFP transgene, but absent from the 6.2 kb-GFP transgene, contain important instructions (perhaps in conjunction with the shorter promoter sequence) for tissue-specific expression in PGCs, skin epidermal cells as well as tissues derived from skin epidermis.

The results presented here demonstrate that the intron-5/6 region of the ship1 gene contains the promoter/enhancer for tissue-specific expression in primitive embryonic cell populations. GFP expression by the 11.5 kb-GFP construct was first observed in cultured ES cells, then in all blastomeres of the morula, and in the ICM of the blastocyst from Tg11.5 kb-GFP mice. Thus the initial embryo expression occurred uniformly in the totipotent cells of the preimplantation embryo. Following implantation, the initially GFP⁺ epiblast lost GFP expression; however, at E7.5 a few cells in the extraembryonic membranes and placenta were GFP⁺. Observations at different times indicated these cells originated near the epiblast, and probably gave rise to the few GFP⁺ yolk sac cells seen in the blood islands beginning at E8.5. The embryo proper lacked GFP expression from E7.5 to about E10.5, but strong GFP expression was observable around E11.5 in skin epidermis, mammary buds, developing gonads, limb AER region, and a few days later, the developing hair follicles. From E15.5-18.5 the AER GFP label (and structure) vanished, but skin epidermal cells retained GFP expression. During this time, however, GFP⁺ cells of the skin appendages were retained in a small cluster (hair follicles) or a few peripheral epithelial cells (mammary tissue). Also, vSMCs, osteoblasts of developing bone, the vomeronasal organ, prostate, and a few cells of the thymus were GFP⁺ at E15.5-18.5.

Several significant points can be made about the embryonic expression pattern of the 11.5 kb-GFP transgene. First, a strong preference exists for stem/progenitor cells (ES cells, morula, ICM, primordial germ cells, epidermis), but also for several cell types of yet undefined character and potential (extraembryonic cells, yolk sac cells, thymus, vemeronasal cells, vSMCs, osteoblasts). Second, no clear continuum of GFP-expressing cells is observed throughout embryo development; rather, it is likely that transgene expression is turned on and off at various stages and locations during development. Third, as observed in the Tg11.5 kb-GFP vs. the Tg6.2 kb-GFP mice, distinct portions of the intron5/6 promoter/enhancer are essential for tissue-specific expression. Finally, transgene GFP expression was never observed in more mature cells of either embryonic or adult tissues, and many of the GFP⁺ stem/progenitor cells in the embryo are also retained in the adult tissues (ms in preparation). These results indicate that s-SHIP expression is spatially and temporally regulated throughout development (see supplementary Table 5).

TABLE 5
Summary of temporal and spatial GFP expression in the
Tg11.5kb-GFP and Tg6.2kb-GFP mouse embryos.

| Embryo Age | Expression Site | | Tg11.5kb-GFP | Tg6.2kb-GFP |
|---|---|---|---|---|
| F2.5 | morula | | +++ | ++++ |
| E3.5 | blastula: | | | |
| | ICM | | +++ | +++ |
| | trophect | | +/− | ++ |
| E6 | epiblast | | +++ | |
| | extra embryonic | | +/− | |
| E7.5 | epiblast | | +/− | |
| | extra embryonic | | ++ | +++ |
| E8.5 | yolk sac | | ++ | |
| | placenta | | ++ | |
| | PGC (migrating) | | − | |
| E9.5 | PGC (migrating) | | − | |
| E11.5 | PGC (genital | | ++ | − |
| | ridge) | | +++ | − |
| | skin epidermis | | +++ | − |
| | hair follicles | | +++ | − |
| | mammary buds | | +++ | − |
| | AER | | +++ | − |
| E13.5 | gonads | | +++ | − |
| | epidermis | | +++ | − |
| E15.5 | gonads: | testis | +++ | |
| | | ovary | +++ | − |
| | skin epidermis | | +++ | − |
| | mammary tissue | | +++ | − |
| | thymus | | + | − |
| | blood vessels (SMC) | | +/− | ++ |
| E17.5-18.5 | gonads: | testis | +++ | − |
| | | ovary | ++ | |

| | | |
|---|---|---|
| skin epidermis | +++ | − |
| hair follicles | +++ | − |
| mammary tissue | +++ | − |
| prostate | +++ | |
| thymus | + | |
| blood vessels (SMC) | + | +++ |
| osteoblasts | ++ | +++ |

Although several tissues expressed the GFP transgene, most tissues and organs did not, and these include tissues with defined or postulated stem/progenitor cell activity, such as muscle, pancreas, small intestine and colon (Marshak et al., 2001; Charge and Rudnicki, 2003; for a contrasting view Dor et al., 2004). Also negative for GFP expression were the E11.5 dorsal aorta and E13.5 fetal liver-sites, respectively, where definitive hematopoiesis is proposed to occur, and where primitive hematopoietic stem cells home and develop (Dzierzak, Medvinsky, and de Bruijn, 1998). This indicates that the 11.5 kb-GFP transgenic GFP promoter/enhancer is highly tissue-specific in its activity.

### Example 4

### Isolation and Characterization of the Human s-SHIP Promoter

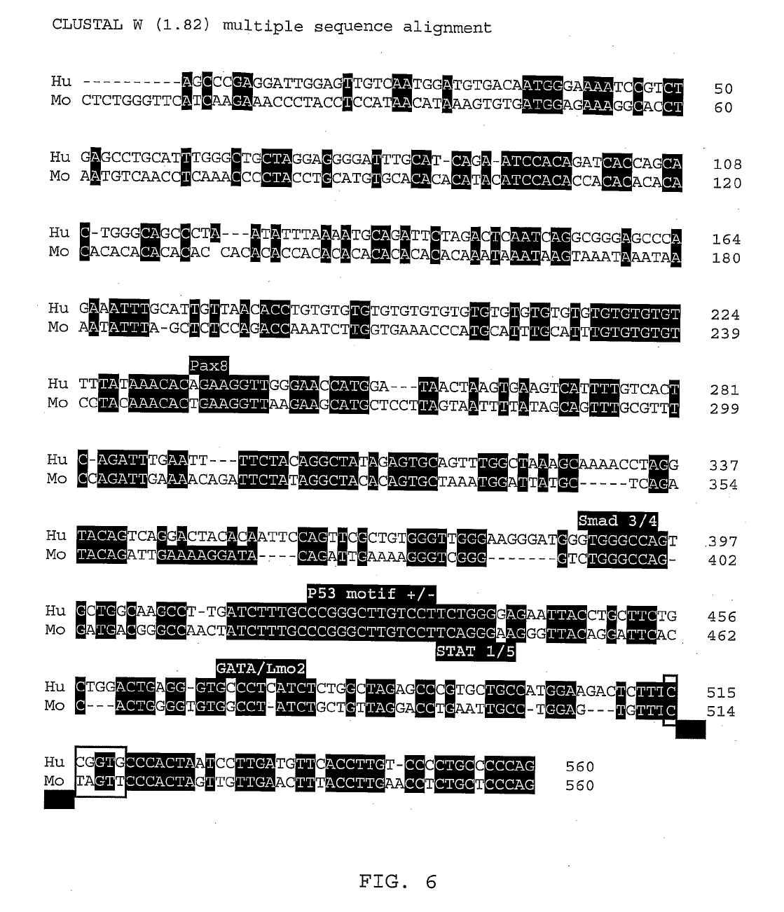

The human s-ship promoter has been isolated and compared to the mouse promoter. FIG. 6 provides a comparison between a region having the genomic sequence of the human promoter that includes 560 nucleotides upstream from exon 6 (at the 3′ end of intron 5) and the corresponding sequence from the mouse sequence.

The mouse and human promoters were significantly homologous. Both promoters contain a binding motif for p53 proteins (p53, p63 or p73). The motif is identified in FIG. 6. Electrophoretic mobility shift assays show that p53 from nuclear extracts of ES cells will bind to a sequence with the p53 motif shown in FIG. 6. FIG. 7 shows p53 binding sequences in mouse, including the different half sites.

In separate experiments, DNA damage caused by UV or gamma irradiation of ES cells induced both p53 and s-SHIP protein expression. ES cells that lack p53, from knockout experiments, did not express nor induce s-SHIP protein after UV irradiation. From the Tg11.5 kb-GFP mice it is known that GFP expression occurs in cells that express p53 (ES cells and others), p63 (epithelial cells of skin, prostate, and mammary tissue), and p73 (neuroepithelial cells as in the vomeronasal organs).

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references are specifically incorporated herein by reference.
U.S. Pat. No. 4,237,224
U.S. Pat. No. 4,683,202
U.S. Pat. No. 4,873,191
U.S. Pat. No. 5,175,384
U.S. Pat. No. 5,175,385
U.S. Pat. No. 5,302,523
U.S. Pat. No. 5,322,783
U.S. Pat. No. 5,384,253
U.S. Pat. No. 5,399,363
U.S. Pat. No. 5,464,765
U.S. Pat. No. 5,466,468
U.S. Pat. No. 5,530,179
U.S. Pat. No. 5,538,877
U.S. Pat. No. 5,538,880
U.S. Pat. No. 5,543,158
U.S. Pat. No. 5,550,318
U.S. Pat. No. 5,563,055
U.S. Pat. No. 5,565,186
U.S. Pat. No. 5,580,859
U.S. Pat. No. 5,589,466
U.S. Pat. No. 5,610,042
U.S. Pat. No. 5,612,486
U.S. Pat. No. 5,616,491
U.S. Pat. No. 5,616,491
U.S. Pat. No. 5,625,125
U.S. Pat. No. 5,639,457
U.S. Pat. No. 5,641,515
U.S. Pat. No. 5,656,610
U.S. Pat. No. 5,702,932
U.S. Pat. No. 5,736,524
U.S. Pat. No. 5,780,448
U.S. Pat. No. 5,789,215
U.S. Pat. No. 5,846,225
U.S. Pat. No. 5,846,233
U.S. Pat. No. 5,925,565
U.S. Pat. No. 5,928,906
U.S. Pat. No. 5,935,819
U.S. Pat. No. 5,945,100
U.S. Pat. No. 5,981,274
U.S. Pat. No. 5,994,624
Abbott et al., J. Nucl. Cardiol., 10(4):403-412, 2003.
Abremski and Hess, J. Mol. Biol., 259:1509-1514, 1984.
Abremski et al., Cell, 32:1301-1311, 1983.
Alanso and Fuchs, Cell, 17:1189-1200, 2003.
Al-Hajj et al., Proc. Natl. Acad. Sci. USA, 100:3983-3988, 2003.
Almendro et al., J. Immunol., 157(12):5411-5421, 1996.
Amin et al., J. Molec. Biol., 214:55-72, 1990.
Anderson et al., J. of Immunotherapy, 12:19-31, 1992.
Andl et al., Cell, 2:643-653, 2002.
Angel et al., Cell, 49:729, 1987b.
Angel et al., Mol. Cell. Biol., 7:2256, 1987a.
Arap et al., Cancer Res., 55(6):1351-1354, 1995.
Armentano et al, Proc. Natl. Acad. Sci. USA, 87(16):6141-6145, 1990.
Atchison and Perry, Cell, 46:253, 1986.
Atchison and Perry, Cell, 48:121, 1987.
Ausubel et al., In: Current Protocols in Molecular Biology, John, Wiley & Sons, Inc, NY, 1996.
Ausubel et al., In: Current Protocols in Molecular Biology, John, Wiley & Sons, Inc, NY, 1994.
Ausubel et al., In: Current Protocols in Molecular Biology, John, Wiley & Sons, Inc, NY, 1992.
Baichwal and Sugden, In: Gene Transfer, Kucherlapati (Ed.), NY, Plenum Press, 117-148, 1986.
Bakhshi et al., Cell, 41(3):899-906, 1985.
Banerji et al., Cell, 27(2 Pt 1):299-308, 1981.
Banerji et al., Cell, 33(3):729-740, 1983.
Bellus, J. Macromol. Sci. Pure Appl. Chem., A31(1): 1355-1376, 1994.
Bellus, J. Macromol. Sci. Pure Appl. Chem., A31(1): 1355-1376, 1994.
Berkhout et al., Cell, 59:273-282, 1989.
Bhatia et al, Proc. Natl. Acad. Sci. USA, 94:5320-5325, 1997.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bulla and Siddiqui, J. Virol., 62:1437, 1986.
Butler and Kadonaga, Genes Dev., 16:2583-2592, 2002.
Caldas et al., Nat. Genet., 8(1):27-32, 1994.
Campbell and Villarreal, Mol. Cell. Biol., 8:1993, 1988.
Campere and Tilghman, Genes and Dev., 3:537, 1989.
Campo et al., Nature, 303:77, 1983.
Capaldi et al., Biochem. Biophys. Res. Comm., 74(2):425-433, 1977.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Charge and Rudnicki, Cell, 113:422-423, 2003.
Chatterjee et al., Proc. Natl. Acad. Sci. USA, 86:9114, 1989.
Chen and Okayama, Mol. Cell. Biol., 7(8):2745-2752, 1987.
Cheng et al., Cancer Res., 54(21):5547-5551, 1994.
Choi et al., Cell, 53:519, 1988.
Chowdhury et al., Science, 254(5039):1802-1805, 1991.
Cleary and Sklar, Proc. Natl. Acad. Sci. USA, 82(21):7439-7443, 1985.
Cleary et al., J. Exp. Med., 164(1):315-320, 1986.
Cocea, Biotechniques, 23(5):814-816, 1997.
Cohen et al., J. Cell. Physiol., 5:75, 1987.
Costa et al., Mol. Cell. Biol., 8:81, 1988.
Coupar et al., Gene, 68:1-10, 1988.
Cournoyer et al., Curr. Opin. Biotechnol., 1(2):196-208, 1990.
Craig, Ann. Rev. Genet., 22:77-105, 1988.
Cregg and Madden, Mol. Gen. Genet., 219:320-323, 1989.
Cripe et al., EMBO J., 6:3745, 1987.
Cristiano et al., Proc. Natl. Acad. Sci. USA, 90(24):11548-11552, 1993.
Culotta and Hamer, Mol. Cell. Biol., 9:1376, 1989.
Dai et al., J. Biol. Chem., 267(27):19565-19571, 1992.
Dailey et al., Mol. Cell. Biol., 14:7758-7769, 1994.
Daley et al., Exp. Hematol., 21(6):734-737, 1993.
Dandolo et al., J. Virology, 47:55-64, 1983.
de Villartay, Nature, 335:170-174, 1988.
De Villiers et al., Nature, 312(5991):242-246, 1984.
Deschamps et al., Science, 230:1174-1177, 1985.
Dor et al., Nature, 429:41-46, 2004.
Dzierzak et al., Immunol. Today, 19:228-236, 1998.
Echols, J. Biol. Chem., 265:14697-14700, 1990.
Edbrooke et al., Mol. Cell. Biol., 9:1908, 1989.
Edlund et al., Science, 230:912-916, 1985.
Fechheimer, et al., Proc Natl. Acad. Sci. USA, 84:8463-8467, 1987.
Feng and Holland, Nature, 334:6178, 1988.
Ferry et al., Proc. Natl. Acad. Sci. USA, 88(19):8377-8381, 1991.
Firak and Subramanian, Mol. Cell. Biol., 6:3667, 1986.
Flanagan and Fennewald, J. Molec. Biol., 206:295-304, 1989.
Foecking and Hofstetter, Gene, 45(1):101-105, 1986.
Fraley et al., Proc. Natl. Acad. Sci. USA, 76:3348-3352, 1979.
Friedmann, Science, 244:1275-1281, 1989.
Friedmann, Science, 244:1275-1281, 1989.
Fujita et al., Cell, 49:357, 1987.
Fung-Leung et al., Cell, 65(3):443-449, 1991b.
Fung-Leung et al., J Exp Med., 174(6):1425-1429, 1991a.
Gilles et al., Cell, 33:717, 1983.
Glasgow et al., J. Biol. Chem., 264:10072-10082, 1989.
Gloss et al., EMBO J., 6:3735, 1987.
Godbout et al., Mol. Cell. Biol., 8:1169, 1988.
Golic and Lindquist, Cell, 59:499-509, 1989.
Goodbourn and Maniatis, Proc. Natl. Acad. Sci. USA, 85:1447, 1988.
Goodboun et al., Cell, 45:601, 1986.
Gopal, Mol. Cell. Biol., 5:1188-1190, 1985.
Graham and Van Der Eb, Virology, 52:456-467, 1973.
Greene et al., Immunology Today, 10:272, 1989
Grosschedl and Baltimore, Cell, 41:885, 1985.
Gupta et al., J. Biol. Chem., 274:7489-7494, 1999.
Haffter and Bickle, EMBO J., 7:3991-3996, 1988.
Harland and Weintraub, J. Cell Biol., 101(3):1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Natl. Acad. Sci. USA, 81:6466-6470, 1984.
Herr and Clarke, Cell, 45:461, 1986.
Herz and Gerard, Proc. Natl. Acad. Sci. USA, 90:2812-2816, 1993.
Hirai, Hum Cell., 15(4):190-198, 2002.
Hirochika et al., J. Virol., 61:2599, 1987.
Hirsch et al., Mol. Cell. Biol., 10:1959, 1990.
Hoess and Abremski, Proc. Natl. Acad. Sci. USA, 81(4):1026-1029, 1984.
Hogan et al., In: Manipulating the Mouse Embryo: A Laboratory Manual, 2nd ed., Cold Spring Harbor Laboratory Press, 1994.
Hogan et al., In: Manipulating the Mouse Embryo: A Laboratory Manual, 2nd ed., Cold Spring Harbor Laboratory Press, 1994.
Holbrook et al., Virology, 157:211, 1987.
Hollestein et al., Science, 253:49-53 1991.
Horlick and Benfield, Mol. Cell. Biol., 9:2396, 1989.
Horn et al., Leukemia, 15:112-120, 2001.
Horwich et al. J. Virol., 64:642-650, 1990.
Huang et al., Cell, 27:245, 1981.
Hubner et al., J. Molec. Biol., 205:493-500, 1989.
Hug et al., Mol. Cell. Biol., 8:3065, 1988.
Hunger-Bertling et al., Mol. Cell. Biochem., 92:107-116, 1990.
Hussussian et al., Nat. Genet., 8(1):15-21, 1994.
Hwang et al., Mol. Cell. Biol., 10:585, 1990.
Hwu et al., J. Immunol., 150(9):4104-4115, 1993.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Inouye and Inouye, Nucleic Acids Res., 13:3101-3109, 1985.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kaeppler et al., Plant Cell Reports, 9:415-418, 1990.
Kamb et al., Nat. Genet., 8(1):23-26, 1994.
Kamb et al., Science, 2674:436-440, 1994.
Kaneda et al., Science, 243:375-378, 1989.
Karin et al., Mol. Cell. Biol., 7:606, 1987.
Karin et al., Mol. Cell. Biol., 7:606, 1987.
Katinka et al., Cell, 20:393, 1980.
Kato et al, J. Biol. Chem., 266:3361-3364, 1991.
Kaufman, In: The Atlas of Mouse Development, Academic Press, San Diego, 2001.
Kavanaugh et al., Current Biol., 6:438-445, 1996.
Kawamoto et al., Mol. Cell. Biol., 8:267, 1988.
Kay et al., Hum. Gene Ther., 3(6):641-647, 1992.
Kerr et al., Br. J. Cancer, 26(4):239-257, 1972.
Kiledjian et al., Mol. Cell. Biol., 8:145, 1988.
Klamut et al., Mol. Cell. Biol., 10:193, 1990.
Koch et al., Mol. Cell. Biol, 9:303, 1989.
Komori, et al., Cell, 89:755-764, 1997.
Kondo et al., Annu. Rev. Immunol., 21:759-806., 2003.
Kozak, Nucleic Acids Res., 15:8125-8148, 1987.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunkel et al., Methods Enzymol., 154:367-382, 1987.
Kunz et al., Nucl. Acids Res., 17:1121, 1989.
Lapidot et al, Nature, 367:645-648, 1994.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., Biochem. Biophys. Res. Commun., 240(2):309-313, 1997.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Lin et al., Mol. Cell. Biol., 10:850, 1990.
Lioubin et al., Genes Devel., 10:1084-1095, 1996.
Lioubin et al., Mol Cell Biol., 14(9):5682-5691, 1994.
Liu et al., Blood, 91:2753-2759, 1998.
Liu et al., Mol. Cell. Biol., 21:3047-3056, 2001.
Long, et al., Development, 131:1309-1318, 2004.
Lucas and Rohrschneider, Blood, 93:1922-1933, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Majors and Varmus, Proc. Natl. Acad. Sci. USA, 80:5866, 1983.
Malynn et al., Cell, 54:453-460, 1988.
Marshak et al., In: Stem Cell Biology, Cold Harbor Laboratory Press, Cold Spring Harbor, N.Y., 2001.
Marsters et al., Recent Prog. Homm. Res., 54:225-234, 1999.
Matsuzaki et al., J. Bacteriol., 172:610-618, 1990.
McNeall et al., Gene, 76:81, 1989.
Mercier et al., J. Bacteriol., 172:3745-757, 1990.
Miksicek et al., Cell, 46:203, 1986.
Miller et al., Am. J. Clin. Oncol., 15(3):216-221, 1992.
Miller, J. Invest. DermatoL, 118:216-225, 2002.
Mordacq and Linzer, Genes and Dev., 3:760, 1989.
Moreau et al., Nucl. Acids Res., 9:6047, 1981.
Mori et al., Cancer Res., 54(13):3396-3397, 1994.
Muesing et al., Cell, 48:691, 1987.
Nagy et al., In: Manipulating the Mouse Embryo, A Laboratory Manual, Third Ed. Cold Spring Harbor Press, 2003.
Nakano et al., Science, 265:1098-1101, 1994.
Nakano, Trends Immunol., 24(11):589-594, 2003.
Ng et al., Nuc. Acids Res., 17:601, 1989.
Nicolas and Rubenstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Nobri et al., Nature (London), 368:753-756, 1995.
Nomoto et al., Gene, 236(2):259-271, 1999.
Nott et al., Genes Dev., 18:210-222, 2004.
Okamoto et al., Proc. Natl. Acad. Sci. USA, 91(23):11045-11049, 1994.
Ondek et al., EMBO J., 6:1017, 1987.
Orlow et al., Cancer Res, 54(11):2848-2851, 1994.
Ornitz et al., Mol. Cell. Biol., 7:3466, 1987.
Palmiter et al., Nature, 300:611, 1982.
Parsons et al., J. Biol. Chem., 265:4527-4533, 1990.
PCT Appln. WO 94/09699
PCT Appln. WO95/06128
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Pesce et al., Bioessays, 20:722-732, 1998.
Pfeffer et al., Cell, 73(3):457-467, 1993.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potrykus et al., Mol. Gen. Genet., 199:183-188, 1985.
Poyart-Salneron et al., EMBO J., 8:2425-2433, 1989.
Quantin et al., Proc. Natl. Acad. Sci. USA, 89(7):2581-2584, 1992.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Resendez Jr. et al, Mol. Cell. Biol., 8:4579, 1988.
Ridgeway, In: Vectors: A survey of molecular cloning vectors and their uses, Rodriguez and Denhardt (Eds.), Stoneham:Butterworth, 467-492, 1988.
Ripe et al., Mol. Cell. Biol., 9:2224, 1989.
Rippe et al., Mol. Cell. Biol., 10:689-695, 1990.
Rittling et al., Nuc. Acids Res., 17:1619, 1989.
Rohrschneider et al., Genes Devel., 14:505-520, 2000.
Rohrschneider, In: Handbook of Cell Signaling, Bradshaw and Dennis (Eds.), 148(2):147-151, Elsevier Sciences (USA), 2003.
Rosen et al., Cell, 41:813, 1988.
Rosenfeld et al., Cell, 68:143-155, 1992.
Sakai et al., Genes and Dev., 2:1144, 1988.
Sambrook et al., In: Molecular cloning, Cold Spring Harbor Laboratory Press, Cold Spring Harbor, N.Y., 1989.
Sanchez, et al., Development, 126:3891-3904, 1999.
Satake et al., J. Virology, 62:970, 1988.
Sato et al., J. Bacteriol., 172:1092-1098, 1990.
Sattler et al., J. Biol. Chem., 276:2451-2458, 2001.
Schaffner et al., J. Mol. Biol., 201:81, 1988. Schwartz and Sadowski, J. Molec. Biol., 205:647-658, 1989.
Searle et al., Mol. Cell. Biol., 5:1480, 1985.
Serrano et al., Nature, 366:704-707, 1993.
Serrano et al., Science, 267(5195):249-252, 1995.
Sharp and Marciniak, Cell, 59:229, 1989.
Shaul and Ben-Levy, EMBO J., 6:1913, 1987.
Sherman et al., Mol. Cell. Biol., 9:50, 1989.
Singh et al., Cancer Res., 63:5821-5828, 2003.
Sleigh and Lockett, J. EMBO, 4:3831, 1985.
Sly et al., Exp. Hematol., 31:1170-1181, 2003.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J, 248:1, 1987.
Sternberg et al. Cold Spring Harbor Symp. Quant. Biol. 45:297-309, 1981.
Stuart et al., Nature, 317:828, 1985.
Sullivan and Peterlin, Mol. Cell. Biol., 7:3315, 1987. Swartzendruber and Lehinan, J. Cell. Physiology, 85:179, 1975.
Takebe et al., Mol. Cell. Biol., 8:466, 1988.
Tamir et al., Immunity, 12:347-358, 2000.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Tomic et al., Nuc. Acids Res., 12:1656, 1990.
Treisman, Cell, 42:889, 1985.
Tronche et al., Mol. Biol. Med., 7:173, 1990. Trudel and Constantini, Genes and Dev. 6:954, 1987.
Tsujimoto and Croce, Proc. Natl. Acad. Sci. USA, 83(14):5214-5218, 1986.
Tsujimoto et al., Nature, 315:340-343, 1985.
Tsumaki et al., J. Biol. Chem., 273(36):22861-22864, 1998.
Tu et al., Blood, 98:2028-2038, 2001.
Tyndell et al., Nuc. Acids. Res., 9:6231, 1981.
Upender et al., Biotechniques, 18:29-31, 1995.
Van Beusechem et al., Proc. Natl. Acad. Sci. USA, 89(16):7640-7644, 1992.
Vannice and Levinson, J. Virology, 62:1305, 1988.
Vasseur et al., Proc Natl. Acad. Sci. U.S.A., 77:1068, 1980.
Wang and Calame, Cell, 47:241, 1986.
Watson and Ramstad, In: Corn: Chemistry and Technology, 1987.
Weber et al., Cell, 36:983, 1984.
Weinberg, Science, 254(5035):1138-1146, 1991.
Weinberger et al. Mol. Cell. Biol., 8:988, 1984.
Weisberg et al., In: Lambda II, Hendrix et al. (Eds.), Cold Spring Harbor Press, Cold Spring Harbor, N.Y., 211-250, 1983.
Winoto and Baltimore, Cell 59:649, 1989.
Wolf et al., Genomics, 69:104-112, 2002.
Wolff et al., Science, 246:1464-1468, 1990.
Wong et al., Gene, 10:87-94, 1980.
Wu et al., Biochem. Biophys. Res. Commun., 233(1):221-6, 1997.
Yutzey et al. Mol. Cell. Biol., 9:1397, 1989.
Zhao-Emonet et al., Biochim. Biophys. Acta, 1442(2-3):109-119, 1998.

1. An isolated polynucleotide comprising an s-ship promoter capable of promoting transcription operably connected to a heterologous nucleic acid sequence.

2.-3. (canceled)

4. The isolated polynucleotide of claim 1, wherein the promoter comprises at least 100 nucleotides from SEQ ID NO:1, SEQ ID NO:2, SEQ ID NO:3, SEQ ID NO:4, or SEQ ID NO:5.

5. (canceled)

6. The isolated polynucleotide of claim 1, wherein the promoter comprises at least 1000 nucleotides from SEQ ID NO:1, SEQ ID NO:2, or SEQ ID NO:5.

7. The isolated polynucleotide of claim 6, wherein the promoter comprises at least 5000 nucleotides from SEQ ID NO:1, SEQ ID NO:2, or SEQ ID NO:5.

8. The isolated polynucleotide of claim 7, wherein the promoter comprises at least 6.3 kilobases from SEQ ID NO:1 or SEQ ID NO:5.

9. The isolated polynucleotide of claim 8, wherein the promoter comprises at least 7.6 kilobases from SEQ ID NO:1 or SEQ ID NO:5.

10. The isolated polynucleotide of claim 1, comprising a sequence that hybridizes under stringent conditions to the complement of SEQ ID NO:2, SEQ ID NO:3, SEQ ID NO:4 or SEQ ID NO:5.

11. The isolated polynucleotide of claim 10, comprising SEQ ID NO:2, SEQ ID NO:3, SEQ E) NO:4 or SEQ E) NO:5.

12. The isolated polynucleotide of claim 1, wherein the promoter is capable of promoting tissue-specific transcription.

13. (canceled)

14. The isolated polynucleotide of claim 1, comprising a sequence that can hybridize under stringent conditions to nucleic acid segment comprising the complement of i) at least 50 contiguous nucleic acids of SEQ ID NO:1, SEQ ID NO:2, SEQ ID NO:3, SEQ ID NO:4, or SEQ ID NO:5.

15. A nucleic acid comprising a promoter operably attached to a nucleic acid sequence from an s-ship gene or a portion thereof and a marker sequence, wherein the s-ship gene is disrupted by the marker sequence.

16. The nucleic acid of claim 15, wherein the promoter is an s-ship promoter.

17. The nucleic acid of claim 15, wherein the promoter is constitutive.

18. The nucleic acid of claim 15, wherein the promoter is inducible or conditional.

19. An expression cassette comprising an s-ship promoter operably connected to a heterologous nucleic acid segment.

20. The expression cassette of claim 19, wherein the heterologous nucleic acid segment encode a protein.

21. The expression cassette of claim 20, wherein the nucleic acid segment is a reporter gene.

22. The expression cassette of claim 21, wherein the reporter gene encodes a gene product that is calorimetric, enzymatic, luminescent, or fluorescent.

23. The expression cassette of claim 19, wherein the nucleic acid segment encodes a therapeutic or diagnostic gene product.

24.-28. (canceled)

29. A vector comprising an s-ship promoter.

30. The vector of claim 1, wherein the s-ship promoter is operably attached to a nucleic acid segment.

31. The vector of claim 30, wherein the nucleic acid segment is all or part of an s-ship1 coding sequence.

32. The vector of claim 30, wherein the nucleic acid segment is heterologous.

33. The vector of claim 29, wherein the vector is a plasmid, YAC, BAC, or virus.

34. The vector of claim 29, comprised in a pharmaceutically acceptable formulation.

35. A host cell comprising the expression cassette of claim 19.

36. (canceled)

37. The host cell of claim 35, wherein the host cell is an embryonic cell.

38. (canceled)

39. The host cell of claim 35, wherein the host cell is a hematopoietic cell.

40. The host cell of claim 35, wherein the host cell is a stem or progenitor cell.

41.-44. (canceled)

45. A mammal having cells comprising an s-ship transgenic sequence.

46. The mammal of claim 45, wherein the s-ship transgenic sequence comprises a s-ship1 coding sequence flanked by loxP sequences.

47. The mammal of claim 46, further comprising a heterologous nucleic acid sequence encoding a Cre recombinase.

48. The mammal of claim 47, wherein the nucleic acid sequence encoding the Cre recombinase is under the control of an inducible or conditional promoter.

49. (canceled)

50. A method of screening for a candidate substance that regulates activity of the s-ship1 promoter comprising a step selected from the group consisting of: (a) contacting a nucleic acid comprising an s-ship promoter with an s-ship promoter binding protein and the candidate substance under conditions that allow binding between the protein and the promoter and determining whether the candidate compound modulates the binding between the protein and the promoter; and (b) contacting the candidate substance with a cell comprising the s-ship promoter operably attached to a reporter gene coding for an expression product and assaying for expression of the reporter gene expression product.

51. A method for identifying stem cells in a population of cells comprising:
(a) administering to cells in the population a nucleic acid comprising an s-ship promoter operably attached to a reporter gene.

52. The method of claim 51, wherein the cells are in an organ.

53. The method of claim 51, wherein the cell are in an animal.

54. The method of claim 51, further comprising sorting cells based on expression of the reporter gene.

55. A method for screening for a modulator of cell function comprising: a) transfecting a stem or hematopoietic cell with an expression cassette comprising an s-ship promoter operably attached to a nucleic acid encoding a candidate modulator; and, b) assaying the cell for a cell function, wherein a difference in cell function in the cell as compared to a cell in the absence of the candidate modulator is indicative of a modulator.

56. The method of claim 55, wherein the modulator is a candidate therapeutic agent for the treatment of a blood-related disease or condition.

57.-76. (canceled)
